(19) United States
(12) Patent Application Publication
(10) Pub. No.: US 2003/0138375 A1
(43) Pub. Date: Jul. 24, 2003

(54) Individualization of therapy with Alzheimer's disease agents

(75) Inventor: Brian Leyland-Jones, Miami, FL (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: McGill University, Montreal (CA)

(21) Appl. No.: 10/164,854

(22) Filed: Jun. 6, 2002

Related U.S. Application Data
(60) Provisional application No. 60/295,860, filed on Jun. 6, 2001.

(51) Int. Cl. G01N 33/53; A61K 49/00

(52) U.S. Cl. 424/9.1; 435/7.1

(57) ABSTRACT

The invention relates to the individualization of therapy on the basis of a phenotypic profile of an individual. More specifically, the present invention relates to the use of metabolic phenotyping for the individualization of treatment with Alzheimer's disease agents.

### RELATED APPLICATION

This application is a new application which claims the benefit of U.S. Provisional Application No. 60/295,860, filed on Jun. 6, 2001. The entire teachings of the above application are incorporated herein by reference.

### BACKGROUND OF THE INVENTION

The invention relates to a system and method for individualization of therapy with Alzheimer's disease agent. More specifically, the present invention relates to the use of metabolic phenotyping in individualizing treatment with Alzheimer's disease agent.

For the majority of drugs (or xenobiotics) administered to humans, their fate is to be metabolized in the liver, into a form less toxic and lipophilic with their subsequent excretion in the urine. Their metabolism involves two systems (Phase I and Phase II) which act consecutively: Phase I enzymes include the cytochrome P450 system which includes at least 20 enzymes catalyzing oxidation reactions as well as carboxylesterase, amindases, epoxide hydrolase, quinine reductase, alcohol and aldehyde dehydrogenase, xanthine oxidase and flavin-containing monooxygenase. These enzymes are localized in the microsomal fraction. Phase II enzymes include the conjugation system which involves at least 5 enzymes including, N-acetyltransferases (NAT), UDP-glucoronyltransferases (UGT), sulfotransferases (SUT), and glutathione-S-transferases (GST). A detailed description of the complex human drug metabolizing systems is provided in Kumar and Surapaneni (Medicinal Res. Rev. (2001) 21(5):397-411) and patent application WO 01/59127 A2.

The metabolism of a drug and its movement through the body (pharmacokinetics) are important in determining its effects, toxicity, and interactions with other drugs. The three processes governing pharmacokinetics are the absorption of the drug, distribution to various tissues, and elimination of drug metabolites. These processes are intimately coupled to drug metabolism, since a variety of metabolic modifications alter most of the physicochemical and pharmacological properties of drugs, including solubility, binding to receptors, and excretion rates. The metabolic pathways which modify drugs also accept a variety of naturally occurring substrates such as steroids, fatty acids, prostaglandins, leukotrienes, and vitamins. The enzymes in these pathways are therefore important sites of biochemical and pharmacological interaction between natural compounds, drugs, carcinogens, mutagens, and xenobiotics.

It has long been appreciated that inherited differences in drug metabolism lead to drastically different levels of drug efficacy and toxicity among individuals. For drugs with narrow therapeutic indices, or drugs which require bioactivation (such as codeine), these polymorphisms can be critical. Moreover, promising new drugs are frequently eliminated in clinical trials based on toxicities which may only affect a segment of the individuals in a target group. Advances in pharmacogenomics research, of which drug metabolizing enzymes constitute an important part, are promising to expand the tools and information that can be brought to bear on questions of drug efficacy and toxicity (See Evans, W. E. and R. V. Relling (1999) Science 286: 487-491).

Drug metabolic reactions are categorized as Phase I, which functionalize the drug molecule and prepare it for further metabolism, and Phase II, which are conjugative. In general, Phase I reaction products are partially or fully inactive, and Phase II reaction products are the chief excreted species. However, Phase I reaction products are sometimes more active than the original administered drugs; this metabolic activation principle is exploited by pro-drugs (e. g. L-dopa). Additionally, some nontoxic compounds (e. g. atlatoxin, benzo [a] pyrene) are metabolized to toxic intermediates through these pathways. Phase I reactions are usually rate-limiting in drug metabolism. Prior exposure to the compound, or other compounds, can induce the expression of Phase I enzymes however, and thereby increase substrate flux through the metabolic pathways. (See Klassen, C. D.,Amdur, M. O. and J. Doull (1996) Casarett and Doull's Toxicology: The Basic Science of Poisons, McGraw-Hill, New York, N.Y., pp. 113-186; Katzung, B. G. (1995) Basic and Clinical Pharmacology, Appleton and Lange, Norwalk, Conn., pp. 48-59; Gibson, G. G. and Skett, P. (1994) Introduction to Drug Metabolism, Blackie Academic and Professional, London.)

Drug metabolizing enzymes (DMEs) have broad substrate specificities. This can be contrasted to the immune system, where a large and diverse population of antibodies is highly specific for their antigens. The ability of DMEs to metabolize a wide variety of molecules creates the potential for drug interactions at the level of metabolism. For example, the induction of a DME by one compound may affect the metabolism of another compound by the enzyme.

DMEs have been classified according to the type of reaction they catalyze and the cofactors involved. The major classes of Phase I enzymes include, but are not limited to, cytochrome P450 and flavin-containing monooxygenase. Other enzyme classes involved in Phase 1-type catalytic cycles and reactions include, but are not limited to, NADPH cytochrome P450 reductase (CPR), the microsomal cytochrome b5/NADH cytochrome b5 reductase system, the ferredoxin/ferredoxin reductase redox pair, aldo/keto reductases, and alcohol dehydrogenases. The major classes of Phase II enzymes include, but are not limited to, UDP glucuronyltransferase, sulfotransferase, glutathione S-transferase, N-acyltransferase, and N-acetyl transferase.

Cytochrome P450 and P450 Catalytic Cycle-associated Enzymes

Members of the cytochrome P450 superfamily of enzymes catalyze the oxidative metabolism of a variety of substrates, including natural compounds such as steroids, fatty acids, prostaglandins, leukotrienes, and vitamins, as well as drugs, carcinogens, mutagens, and xenobiotics. Cytochromes P450, also known as P450 heme-thiolate proteins, usually act as terminal oxidases in multi-component electron transfer chains, called P450-containing monooxygenase systems. Specific reactions catalyzed include hydroxylation, epoxidation, N-oxidation, sulfooxidation, N-, S-, and dealkylations, desulfation, deamination, and reduction of azo, nitro, and N-oxide groups. These reactions are involved in steroidogenesis of glucocorticoids, cortisols, estrogens, and androgens in animals; insecticide resistance in insects; herbicide resistance and flower coloring in plants; and environmental bioremediation by microorganisms. Cytochrome P450 actions on drugs, carcinogens, mutagens, and xenobiotics can result in detoxification or in conversion of the substance to a more toxic product. Cytochromes P450 are abundant in the liver, but also occur in other tissues; the enzymes are located in microsomes. (Graham-Lorence, S. and Peterson, J. A. (1996) FASEB J. 10: 206-214.)

Four hundred cytochromes P450 have been identified in diverse organisms including bacteria, fungi, plants, and animals (Graham-Lorence, supra). The B-class is found in prokaryotes and fungi, while the E-class is found in bacteria, plants, insects, vertebrates, and mammals. Five subclasses or groups are found within the larger family of E-class cytochromes P450.

All cytochromes P450 use a heme cofactor and share structural attributes. Most cytochromes P450 are 400 to 530 amino acids in length. The secondary structure of the enzyme is about 70% alpha-helical and about 22% beta-sheet. The region around the heme-binding site in the C-terminal part of the protein is conserved among cytochromes P450. A ten amino acid signature sequence in this heme-iron ligand region has been identified which includes a conserved cysteine involved in binding the heme iron in the fifth coordination site. In eukaryotic cytochromes P450, a membrane-spanning region is usually found in the first 15-20 amino acids of the protein, generally consisting of approximately 15 hydrophobic residues followed by a positively charged residue (Graham-Lorence, supra.).

Cytochrome P450 enzymes are involved in cell proliferation and development. The enzymes have roles in chemical mutagenesis and carcinogenesis by metabolizing chemicals to reactive intermediates that form adducts with DNA (Nebert, D. W. and Gonzalez, F. J. (1987) Ann. Rev. Biochem. 56: 945-993). These adducts can cause nucleotide changes and DNA rearrangements that lead to oncogenesis. Cytochrome P450 expression in liver and other tissues is induced by xenobiotics such as polycyclic aromatic hydrocarbons, peroxisomal proliferators, phenobarbital, and the glucocorticoid dexamethasone (Dogra, S. C. et al. (1998) Clin. Exp. Pharmacol. Physiol. 25: 1-9). A cytochrome P450 protein may participate in eye development as mutations in the P450 gene CYP1B1 cause primary congenital glaucoma.

Cytochromes P450 are associated with inflammation and infection. Hepatic cytochrome P450 activities are profoundly affected by various infections and inflammatory stimuli, some of which are suppressed and some induced (Morgan, E. T. (1997) Drug Metab. Rev. 29: 1129-1188). Effects observed in vivo can be mimicked by proinflammatory cytokines and interferons. Autoantibodies to two cytochrome P450 proteins were found in individuals with autoimmune polyenodocrinopathy-candidiasis-ectodermal dystrophy (APECED), a polyglandular autoimmune syndrome.

Mutations in cytochromes P450 have been linked to metabolic disorders, including congenital adrenal hyperplasia, the most common adrenal disorder of infancy and childhood; pseudovitamin deficiency rickets; cerebrotendinous xanthomatosis, a lipid storage disease characterized by progressive neurologic dysfunction, premature atherosclerosis, and cataracts; and an inherited resistance to the anticoagulant drugs coumarin and warfarin (Isselbacher, K. J. et al. (1994) Harrison's Principles of Internal Medicine, McGraw-Hill, Inc. New York, N.Y., pp. 1968-1970; Takeyama, K. et al. (1997) Science 277: 1827-1830; Kitanaka, S. et al. (1998) N. Engl. J. Med. 338: 653-661). Extremely high levels of expression of the cytochrome P450 protein aromatase were found in a fibrolamellar hepatocellular carcinoma from a boy with severe gynecomastia (feminization) (Agarwal, V. R. (1998) J. Clin. Endocrinol. Metab. 83: 1797-1800).

The cytochrome P450 catalytic cycle is completed through reduction of cytochrome P450 by NADPH cytochrome P450 reductase (CPR). Another microsomal electron transport system consisting of cytochrome b5 and NADPH cytochrome b5 reductase has been widely viewed as a minor contributor of electrons to the cytochrome P450 catalytic cycle. However, a recent report by Lamb, D. C. et al. (1999 FEBS Lett. 462: 283-8) identifies a Candida albicans cytochrome P450 (CYP51) which can be efficiently reduced and supported by the microsomal cytochrome b5/NADPH cytochrome b5 reductase system. Therefore, there are likely many cytochromes P450 which are supported by this alternative electron donor system.

Cytochrome b5 reductase is also responsible for the reduction of oxidized hemoglobin (methemoglobin, or ferrihemoglobin, which is unable to carry oxygen) to the active hemoglobin (ferrohemoglobin) in red blood cells. Methemoglobinemia results when there is a high level of oxidant drugs or an abnormal hemoglobin (hemoglobin M) which is not efficiently reduced. Methemoglobinemia can also result from a hereditary deficiency in red cell cytochrome b5 reductase (Reviewed in Mansour, A. and Lurie, A. A. (1993) Am. J. Hematol. 42: 7-12).

Members of the cytochrome P450 family are also closely associated with vitamin D synthesis and catabolism. Vitamin D exists as two biologically equivalent prohormones, ergocalciferol (vitamin D₂), produced in plant tissues and cholecalciferol (vitamin D₃), produced in animal tissues. The latter form, cholecalciferol, is formed upon the exposure of 7-dehydrocholesterol to near ultraviolet light (i.e., 290-310 nm), normally resulting from even minimal periods of skin exposure to sunlight (reviewed in Miller, W. L. and Portale, A. A. (2000) Trends in Endocrinology and Metabolism 11: 315-319).

Both prohormone forms are further metabolized in the liver to 25-hydroxyvitamin D (25(OH)D) by the enzyme 25-hydroxylase. 25(OH)D is the most abundant precursor form of vitamin D which must be further metabolized in the kidney to the active form, 1α,25-dihydroxyvitamin D (1α,25(OH)₂D), by the enzyme 25-hydroxyvitamin D 1α-hydroxylase (1α-hydroxylase). Regulation of 1α,25(OH)₂D production is primarily at this final step in the synthetic pathway. The activity of 1α-hydroxylase depends upon several physiological factors including the circulating level of the enzyme product (1α,25(OH)₂D) and the levels of parathyroid hormone (PTH), calcitonin, insulin, calcium, phosphorus, growth hormone, and prolactin. Furthermore, extrarenal 1α-hydroxylase activity has been reported, suggesting that tissue-specific, local regulation of 1α,25(OH)₂D production may also be biologically important. The catalysis of 1α,25(OH)₂D to 24,25-dihydroxyvitamin D (24,25(OH)₂D), involving the enzyme 25-hydroxyvitamin D 24-hydroxylase (24-hydroxylase), also occurs in the kidney. 24-hydroxylase can also use 25(OH)₂D as a substrate (Shinki, T. et al. (1997) Proc. Natl. Acad. Sci. U. S. A. 94: 12920-12925; Miller, W. L. and Portale, A. A. supra; and references within).

Vitamin D 25-hydroxylase, 1 α-hydroxylase, and 24-hydroxylase are all NADPH-dependent, type I (mitochondrial) cytochrome P450 enzymes that show a high degree of homology with other members of the family. Vitamin D 25-hydroxylase also shows a broad substrate specificity and may also perform 26-hydroxylation of bile acid intermediates and 25,26, and 27-hydroxylation of cholesterol (Dilworth, F. J. et al. (1995) J. Biol. Chem. 270: 16766-16774; Miller, W. L. and Portale, A. A. supra; and references within).

The active form of vitamin D (1 α,25(OH)₂D) is involved in calcium and phosphate homeostasis and promotes the differentiation of myeloid and skin cells. Vitamin D deficiency resulting from deficiencies in the enzymes involved in vitamin D metabolism (e.g., 1 α-hydroxylase) causes hypocalcemia, hypophosphatemia, and vitamin D-dependent (sensitive) rickets, a disease characterized by loss of bone density and distinctive clinical features, including bandy or bow leggedness accompanied by a waddling gait. Deficiencies in vitamin D 25-hydroxylase cause cerebrotendinous xanthomatosis, a lipid-storage disease characterized by the deposition of cholesterol and cholestanol in the Achilles' tendons, brain, lungs, and many other tissues. The disease presents with progressive neurologic dysfunction, including postpubescent cerebellar ataxia, atherosclerosis, and cataracts. Vitamin D 25-hydroxylase deficiency does not result in rickets, suggesting the existence of alternative pathways for the synthesis of 25 (OH) D (Griffin, J. E. and Zerwekh, J. E. (1983) J. Clin. Invest. 72: 1190-1199; Gamblin, G. T. et al. (1985) J. Clin. Invest. 75: 954-960; and W. L. and Portale, A. A. supra).

Ferredoxin and ferredoxin reductase are electron transport accessory proteins which support at least one human cytochrome P450 species, cytochrome P450c27 encoded by the CYP27 gene (Dilworth, F. J. et al. (1996) Biochem. J. 320: 267-71). A Streptomyces sriseus cytochrome P450, CYP104D1, was heterologously expressed in E. coli and found to be reduced by the endogenous ferredoxin and ferredoxin reductase enzymes (Taylor, M. et al. (1999) Biochem. Biophys. Res. Commun. 263: 838-42), suggesting that many cytochrome P450 species may be supported by the ferredoxin/ferredoxin reductase pair. Ferredoxin reductase has also been found in a model drug metabolism system to reduce actinomycin D, an antitumor antibiotic, to a reactive free radical species (Flitter, W. D. and Mason, R. P. (1988) Arch. Biochem. Biophys. 267: 632-9).

Flavin-containing Monooxygenase (FMO)

Flavin-containing monooxygenases (FMO) oxidize the nucleophilic nitrogen, sulfur, and phosphorus heteroatom of an exceptional range of substrates. Like cytochromes P450, FMOs are microsomal and use NADPH and O₂; there is also a great deal of substrate overlap with cytochromes P450. The tissue distribution of FMOs includes liver, kidney, and lung.

There are five different known isoforms of FMO in mammals (FMO1, FMO2, FMO3, FMO4, and FMOS), which are expressed in a tissue-specific manner. The isoforms differ in their substrate specificities and other properties such as inhibition by various compounds and stereospecificity of reaction. FMOs have a 13 amino acid signature sequence, the components of which span the N-terminal two-thirds of the sequences and include the FAD binding region and the FATGY motif which has been found in many N-hydroxylating enzymes (Stehr, M. et al. (1998) Trends Piochem. Sci. 23: 56-57).

Specific reactions include oxidation of nucleophilic tertiary amines to N-oxides, secondary amines to hydroxylamines and nitrones, primary amines to hydroxylamines and oximes, and sulfur containing compounds and phosphines to S-and P-oxides. Hydrazines, iodides, selenides, and boron-containing compounds are also substrates. Although FMOs appear similar to cytochromes P450 in their chemistry, they can generally be distinguished from cytochromes P450 in vitro based on, for example, the higher heat lability of FMOs and the nonionic detergent sensitivity of cytochromes P450; however, use of these properties in identification is complicated by further variation among FMO isoforms with respect to thermal stability and detergent sensitivity.

FMOs play important roles in the metabolism of several drugs and xenobiotics. FMO (FMO3 in liver) is predominantly responsible for metabolizing (S)-nicotine to (S)-nicotine N-1′-oxide, which is excreted in urine. FMO is also involved in S-oxygenation of cimetidine, an H2-antagonist widely used for the treatment of gastric ulcers. Liver-expressed forms of FMO are not under the same regulatory control as cytochrome P450. In rats, for example, phenobarbital treatment leads to the induction of cytochrome P450, but the repression of FMO1.

Endogenous substrates of FMO include cysteamine, which is oxidized to the disulfide, cystamin, and trimethylamine (TMA), which is metabolized to trimethylamine N-oxide. TMA smells like rotting fish, and mutations in the FMO3 isoform lead to large amounts of the malodorous free amine being excreted in sweat, urine, and breath. These symptoms have led to the designation fish-odor syndrome.

Lysyl Oxidase

Lysyl oxidase (lysine 6-oxidase, LO) is a copper-dependent amine oxidase involved in the formation of connective tissue matrices by cross-linking collagen and elastin. LO is secreted as a N-glycosylated precursor protein of approximately 50 kDa and cleaved to the mature form of the enzyme by a metalloprotease, although the precursor form is also active. The copper atom in LO is involved in the transport of electron to and from oxygen to facilitate the oxidative deamination of lysine residues in these extracellular matrix proteins. While the coordination of copper is essential to LO activity, insufficient dietary intake of copper does not influence the expression of the apoenzyme. However, the absence of the functional LO is linked to the skeletal and vascular tissue disorders that are associated with dietary copper deficiency. LO is also inhibited by a variety of semicarbazides, hydrazines, and amino nitrites, as well as heparin. Beta-aminopropionitrile is a commonly used inhibitor. LO activity is increased in response to ozone, cadmium, and elevated levels of hormones released in response to local tissue trauma, such as transforming growth factor-beta, platelet-derived growth factor, angiotensin II, and fibroblast growth factor. Abnormalities in LO activity has been linked to Menkes syndrome and occipital horn syndrome. Cytosolic forms of the enzyme have been implicated in abnormal cell proliferation (reviewed in Rucker, R. B. et al. (1998) Am. J. Clin. Nutr. 67: 996S-1002S and Smith-Mungo. L. I. and Kagan, H. M. (1998) Matrix Biol. 16: 387-398).

Dihydrofolate Reductases

Dihydrofolate reductases (DHFR) are ubiquitous enzymes that catalyze the NADPH-dependent reduction of dihydrofolate to tetrahydrofolate, an essential step in the de novo synthesis of glycine and purines as well as the conversion of deoxyuridine monophosphate (dUMP) to deoxythymidine monophosphate (dTMP). The basic reaction is as follows:

7,8-dihydrofolate+NADPH→5,6,7,8-tetrahydrofolate+NADP⁺

The enzymes can be inhibited by a number of dihydrofolate analogs, including trimethroprim and methotrexate. Since an abundance of TMP is required for DNA synthesis, rapidly dividing cells require the activity of DHFR. The replication of DNA viruses (one example is herpesvirus) also requires high levels of DHFR activity. As a result, drugs that target DHFR have been used for cancer chemotherapy and to inhibit DNA virus replication. (For similar reasons, thymidylate synthetases are also target enzymes.) Drugs that inhibit DHFR are preferentially cytotoxic for rapidly dividing cells (or DNA virus-infected cells) but have no specificity, resulting in the indiscriminate destruction of dividing cells. Furthermore, cancer cells may become resistant to drugs such as methotrexate as a result of acquired transport defects or the duplication of one or more DHFR genes (Stryer, L (1988) Biochemistry. W. H Freeman and Co., Inc. New York. pp. 511-5619).

Aldo/Keto Reductases

Aldo/keto reductases are monomeric NADPH-dependent oxidoreductases with broad substrate specificities (Bohren, K. M. et al. (1989) J. Biol. Chem. 264: 9547-51). These enzymes catalyze the reduction of carbonyl-containing compounds, including carbonyl-containing sugars and aromatic compounds, to the corresponding alcohols. Therefore, a variety of carbonyl-containing drugs and xenobiotics are likely metabolized by enzymes of this class.

One known reaction catalyzed by a family member, aldose reductase, is the reduction of glucose to sorbitol, which is then further metabolized to fructose by sorbitol dehydrogenase. Under normal conditions, the reduction of glucose to sorbitol is a minor pathway. In hyperglycemic states, however, the accumulation of sorbitol is implicated in the development of diabetic complications. Members of this enzyme family are also highly expressed in some liver cancers (Cao, D. et al. (1998) J. Biol. Chem. 273: 11429-35).

Alcohol Dehydrogenases

Alcohol dehydrogenases (ADHs) oxidize simple alcohols to the corresponding aldehydes. ADH is a cytosolic enzyme, prefers the cofactor NAD+, and also binds zinc ion. Liver contains the highest levels of ADH, with lower levels in kidney, lung, and the gastric mucosa.

Known ADH isoforms are dimeric proteins composed of 40 kDa subunits. There are five known gene loci which encode these subunits (a, b, g, p, c), and some of the loci have characterized allelic variants (b″b2, b3, gl, g2). The subunits can form homodimers and heterodimers; the subunit composition determines the specific properties of the active enzyme. The holoenzymes have therefore been categorized as Class I (subunit compositions aa, ab, ag, bg, gg), Class II (pp), and Class III (cc). Class I ADH isozymes oxidize ethanol and other small aliphatic alcohols, and are inhibited by pyrazole. Class II isozymes prefer longer chain aliphatic and aromatic alcohols, are unable to oxidize methanol, and are not inhibited by pyrazole. Class III isozymes prefer even longer chain aliphatic alcohols (five carbons and longer) and aromatic alcohols, and are not inhibited by pyrazole.

The short-chain alcohol dehydrogenases include a number of related enzymes with a variety of substrate specificities. Included in this group are the mammalian enzymes D-beta-hydroxybutyrate dehydrogenase, (R)-3-hydroxybutyrate dehydrogenase, 15-hydroxyprostaglandin dehydrogenase, NADPH-dependent carbonyl reductase, corticosteroid 11-beta-dehydrogenase, and estradiol 17-beta-dehydrogenase, as well as the bacterial enzymes acetoacetyl-CoA reductase, glucose 1-dehydrogenase, 3-beta-hydroxysteroid dehydrogenase, 20-beta-hydroxysteroid dehydrogenase, ribitol dehydrogenase, 3-oxoacyl reductase, 2,3-dihydro-2,3-dihydroxybenzoate dehydrogenase, sorbitol-6-phosphate 2-dehydrogenase, 7-alpha-hydroxysteroid dehydrogenase, cis-1,2-dihydroxy-3,4-cyclohexadiene-1-carboxylate dehydrogenase, cis-toluene dihydrodiol dehydrogenase, cis-benzene glycol dehydrogenase, biphenyl-2,3-dihydro-2,3-diol dehydrogenase, N-acylmannosamine 1-dehydrogenase, and 2-deoxy-D-gluconate 3-dehydrogenase (Krozowski, z. (1994) J. Steroid Biochem. Mol. Biol. 51: 125-130; Krozowski, Z. (1992) Mol. Cell Endocrinol. 84: C25-31; and Marks, A. R. et al. (1992) J. Biol. Chem. 267: 15459-15463).

Udp Glucuronyltransferase

Members of the UDP glucuronyltransferase family (UGTS) catalyze the transfer of a glucuronic acid group from the cofactor uridine diphosphate-glucuronic acid (UDP-glucuronic acid) to a substrate. The transfer is generally to a nucleophilic heteroatom (O, N, or S). Substrates include xenobiotics which have been functionalized by Phase I reactions, as well as endogenous compounds such as bilirubin, steroid hormones, and thyroid hormones. Products of glucuronidation are excreted in urine if the molecular weight of the substrate is less than about 250 g/mol, whereas larger glucuronidated substrates are excreted in bile.

UGTs are located in the microsomes of liver, kidney, intestine, skin, brain, spleen, and nasal mucosa, where they are on the same side of the endoplasmic reticulum membrane as cytochrome P450 enzymes and flavin-containing monooxygenases, and therefore are ideally located to access products of Phase I drug metabolism. UGTs have a C-terminal membrane-spanning domain which anchors them in the endoplasmic reticulum membrane and a conserved signature domain of about 50 amino acid residues in their C terminal section.

UGTs involved in drug metabolism are encoded by two gene families, UGT1 and UGT2. Members of the UGT1 family result from alternative splicing of a single gene locus, which has a variable substrate binding domain and constant region involved in cofactor binding and membrane insertion. Members of the UGT2 family are encoded by separate gene loci, and are divided into two families, UGT2A and UGT2B. The 2A subfamily is expressed in olfactory epithelium, and the 2B subfamily is expressed in liver microsomes. Mutations in UGT genes are associated with hyperbilirubinemia; Crigler-Najjar syndrome, characterized by intense hyperbilirubinemia from birth; and a milder form of hyperbilirubinemia termed Gilbert's disease.

Sulfotransferase

Sulfate conjugation occurs on many of the same substrates which undergo O-glucuronidation to produce a highly water-soluble sulfuric acid ester. Sulfotransferases (ST) catalyze this reaction by transferring SO₃-from the cofactor 3′-phosphoadenosine-5′-phosphosulfate (PAPS) to the substrate. ST substrates are predominantly phenols and aliphatic alcohols, but also include aromatic amines and aliphatic amines, which are conjugated to produce the corresponding sulfamates. The products of these reactions are excreted mainly in urine.

STs are found in a wide range of tissues, including liver, kidney, intestinal tract, lung, platelets, and brain. The enzymes are generally cytosolic, and multiple forms are often co-expressed. For example, there are more than a dozen forms of ST in rat liver cytosol. These biochemically characterized STs fall into five classes based on their substrate preference: arylsulfotransferase, alcohol sulfotransferase, estrogen sulfotransferase, tyrosine ester sulfotransferase, and bile salt sulfotransferase.

ST enzyme activity varies greatly with sex and age in rats. The combined effects of developmental cues and sex-related hormones are thought to lead to these differences in ST expression profiles, as well as the profiles of other DMEs such as cytochromes P450. Notably, the high expression of STs in cats partially compensates for their low level of UDP glucuronyltransferase activity.

Several forms of ST have been purified from human liver cytosol and cloned. There are two phenol sulfotransferases with different thermal stabilities and substrate preferences. The thermostable enzyme catalyzes the sulfation of phenols such as para-nitrophenol, minoxidil, and acetaminophen; the thermolabile enzyme prefers monoamine substrates such as dopamine, epinephrine, and levadopa. Other cloned STs include an estrogen sulfotransferase and an N-acetylglucosamine-6-O-sulfotransferase. This last enzyme is illustrative of the other major role of STs in cellular biochemistry, the modification of carbohydrate structures that may be important in cellular differentiation and maturation of proteoglycans. Indeed, an inherited defect in a sulfotransferase has been implicated in macular corneal dystrophy, a disorder characterized by a failure to synthesize mature keratan sulfate proteoglycans (Nakazawa, K. et al. (1984) J. Biol. Chem. 259: 13751-7).

Galactosyltransferases

Galactosyltransferases are a subset of glycosyltransferases that transfer galactose (Gal) to the terminal N-acetylglucosamine (GlcNAc) oligosaccharide chains that are part of glycoproteins or glycolipids that are free in solution (Kolbinger, F. et al. (1998) J. Biol. Chem. 273: 433-440; Amado, M. et al. (1999) Biochim. Biophys. Acta 1473: 35-53). Galactosyltransferases have been detected on the cell surface and as soluble extracellular proteins, in addition to being present in the Golgi. β1,3-galactosyltransferases form Type I carbohydrate chains with Gal (β1-3) GlcNAc linkages. Known human and mouse β1,3-galactosyltransferases appear to have a short cytosolic domain, a single transmembrane domain, and a catalytic domain with eight conserved regions. (Kolbinger, F. supra and Hennet, T. et al. (1998) J. Biol. Chem. 273: 58-65). In mouse UDP-galactose: β-N-acetylglucosamine β1,3-galactosyltransferase-I region 1 is located at amino acid residues 78-83, region 2 is located at amino acid residues 93-102, region 3 is located at amino acid residues 116-119, region 4 is located at amino acid residues 147-158, region 5 is located at amino acid residues 172-183, region 6 is located at amino acid residues 203-206, region 7 is located at amino acid residues 236-246, and region 8 is located at amino acid residues 264-275. A variant of a sequence found within mouse UDP-galactose:β-N-acetylglucosamine β1,3-galactosyltransferase-I region 8 is also found in bacterial galactosyltransferases, suggesting that this sequence defines a galactosyltransferase sequence motif (Hennet, T. supra). Recent work suggests that brainiac protein is a β1,3-galactosyltransferase. (Yuan, Y. et al. (1997) Cell 88: 9-11; and Hennet, T. supra).

UDP-Gal:GlcNAc-1,4-galactosyltransferase (-1,4-GalT) (Sato, T. et al., (1997) EMBO J. 16: 1850-1857) catalyzes the formation of Type II carbohydrate chains with Gal (β1-4) GlcNAc linkages. As is the case with the β1,3-galactosyltransferase, a soluble form of the enzyme is formed by cleavage of the membrane-bound form. Amino acids conserved among β1,4-galactosyltransferases include two cysteines linked through a disulfide-bonded and a putative UDPgalactose-binding site in the catalytic domain (Yadav, S. and Brew, K. (1990) J. Biol. Chem. 265: 14163-14169; Yadav, S. P. and Brew, K. (1991) J. Biol. Chem. 266: 698-703; and Shaper, N. L. et al. (1997) J. Biol. Chem. 272: 31389-31399). β1,4-galactosyltransferases have several specialized roles in addition to synthesizing carbohydrate chains on glycoproteins or glycolipids. In mammals a β1,4-galactosyltransferase, as part of a heterodimer with cc-lactalbumin, functions in lactating mammary gland lactose production. A β1,4-galactosyltransferase on the surface of sperm functions as a receptor that specifically recognizes the egg. Cell surface β1,4-galactosyltransferases also function in cell adhesion, cell/basal lamina interaction, and normal and metastatic cell migration (Shur, B. (1993) Curr. Opin. Cell Biol. 5: 854-863; and Shaper, J. (1995) Adv. Exp. Med. Biol. 376: 95-104).

Glutathione S-transferase

The basic reaction catalyzed by glutathione S-transferases (GST) is the conjugation of an electrophile with reduced glutathione (GSH). GSTs are homodimeric or heterodimeric proteins localized mainly in the cytosol, but some level of activity is present in microsomes as well. The major isozymes share common structural and catalytic properties; in humans they have been classified into four major classes, Alpha, Mu, Pi, and Theta. The two largest classes, Alpha and Mu, are identified by their respective protein isoelectric points; pI˜7.5-9.0 (Alpha), and pI˜6.6 Mu). Each GST possesses a common binding site for GSH and a variable hydrophobic binding site. The hydrophobic binding site in each isozyme is specific for particular electrophilic substrates. Specific amino acid residues within GSTs have been identified as important for these binding sites and for catalytic activity. Residues Q67, T68, D101, E104, and R131 are important for the binding of GSH (Lee, H-C et al. (1995) J. Biol. Chem. 270: 99-109). Residues R13, R20, and R69 are important for the catalytic activity of GST (Stenberg G et al. (1991) Biochem. J. 274: 549-55).

In most cases, GSTs perform the beneficial function of deactivation and detoxification of potentially mutagenic and carcinogenic chemicals. However, in some cases their action is detrimental and results in activation of chemicals with consequent mutagenic and carcinogenic effects. Some forms of rat and human GSTs are reliable preneoplastic markers that aid in the detection of carcinogenesis. Expression of human GSTs in bacterial strains, such as Salmonella typhimurium used in the well-known Ames test for mutagenicity, has helped to establish the role of these enzymes in mutagenesis. Dihalomethanes, which produce liver tumors in mice, are believed to be activated by GST. This view is supported by the finding that dihalomethanes are more mutagenic in bacterial cells expressing human GST than in untransfected cells (Thier, R. et al. (1993) Proc. Natl. Acad. Sci. USA 90: 8567-80). The mutagenicity of ethylene dibromide and ethylene dichloride is increased in bacterial cells expressing the human Alpha GST, Al-1, while the mutagenicity of allatoxin Bi is substantially reduced by enhancing the expression of GST (Simula, T. P. et al. (1993) Carcinogenesis 14: 1371-6). Thus, control of GST activity may be useful in the control of mutagenesis and carcinogenesis.

GST has been implicated in the acquired resistance of many cancers to drug treatment, the phenomenon known as multi-drug resistance (MDR). MDR occurs when a cancer individual is treated with a cytotoxic drug such as cyclophosphamide and subsequently becomes resistant to this drug and to a variety of other cytotoxic agents as well. Increased GST levels are associated with some of these drug resistant cancers, and it is believed that this increase occurs in response to the drug agent which is then deactivated by the GST catalyzed GSH conjugation reaction. The increased GST levels then protect the cancer cells from other cytotoxic agents which bind to GST. Increased levels of Al-1 in tumors has been linked to drug resistance induced by cyclophosphamide treatment (Dirven H. A. et al. (1994) Cancer Res. 54: 6215-20). Thus control of GST activity in cancerous tissues may be useful in treating MDR in cancer individuals.

Gamma-glutamyl Transpeptidase

Gamma-glutamyl transpeptidases are ubiquitously expressed enzymes that initiate extracellular glutathione (GSH) breakdown by cleaving gamma-glutamyl amide bonds. The breakdown of GSH provides cells with a regional cysteine pool for biosynthetic pathways. Gamma-glutamyl transpeptidases also contribute to cellular antioxidant defenses and expression is induced by oxidative stress. The cell surface-localized glycoproteins are expressed at high levels in cancer cells. Studies have suggested that the high level of gamma-glutamyl transpeptidases activity present on the surface of cancer cells could be exploited to activate precursor drugs, resulting in high local concentrations of anticancer therapeutic agents (Hanigan, M. H. (1998) Chem. Biol. Interact. 111-112: 333-42; Taniguchi, N. and Ikeda, Y. (1998) Adv. Enzymol. Relat. Areas Mol. Biol. 72: 239-78; Chikhi, N. et al. (1999) Comp. Biochem. Physiol. B. Biochem. Mol. Biol. 122: 367-80).

Acyltransferase

N-acyltransferase enzymes catalyze the transfer of an amino acid conjugate to an activated carboxylic group. Endogenous compounds and xenobiotics are activated by acyl-CoA synthetases in the cytosol, microsomes, and mitochondria. The acyl-CoA intermediates are then conjugated with an amino acid (typically glycine, glutamin, or taurine, but also ornithine, arginine, histidine, serine, aspartic acid, and several dipeptides) by N-acyltransferases in the cytosol or mitochondria to form a metabolite with an amide bond. This reaction is complementary to O-glucuronidation, but amino acid conjugation does not produce the reactive and toxic metabolites which often result from glucuronidation.

One well-characterized enzyme of this class is the bile acid-CoA: amino acid N-acyltransferase (BAT) responsible for generating the bile acid conjugates which serve as detergents in the gastrointestinal tract (Falany, C. N. et al. (1994) J. Biol. Chem. 269: 19375-9; Johnson, M. R. et al. (1991) J. Biol. Chem. 266: 10227-33). BAT is also useful as a predictive indicator for prognosis of hepatocellular carcinoma individuals after partial hepatectomy (Furutani, M. et al. (1996) Hepatology 24: 1441-5).

Acetyltransferases

Acetyltransferases have been extensively studied for their role in histone acetylation. Histone acetylation results in the relaxing of the chromatin structure in eukaryotic cells, allowing transcription factors to gain access to promoter elements of the DNA templates in the affected region of the genome (or the genome in general). In contrast, histone deacetylation results in a reduction in transcription by closing the chromatin structure and limiting access of transcription factors. To this end, a common means of stimulating cell transcription is the use of chemical agents that inhibit the deacetylation of histones (e. g., sodium butyrate), resulting in a global (albeit artifactual) increase in gene expression. The modulation of gene expression by acetylation also results from the acetylation of other proteins, including but not limited to, p53, GATA-1, MyoD, ACTR, TFIIE, TFIIF and the high mobility group proteins (HMG). In the case of p53, acetylation results in increased DNA binding, leading to the stimulation of transcription of genes regulated by p53. The prototypic histone acetylase (HAT) is Gcn5 from Saccharomyces cerevisiae. Gcn5 is a member of a family of acetylases that includes Tetrahymena p55, human GcnS, and human p300/CBP. Histone acetylation is reviewed in (Cheung, W. L. et al. (2000) Current Opinion in Cell Biology 12: 326-333 and Berger, S. L (1999) Current Opinion in Cell Biology 11: 336-341). Some acetyltransferase enzymes posses the alpha/beta hydrolase fold common to several other major classes of enzymes, including but not limited to, acetylcholinesterases and carboxylesterases.

N-acetyltransferase

Aromatic amines and hydrazine-containing compounds are subject to N-acetylation by the N-acetyltransferase enzymes of liver and other tissues. Some xenobiotics can be O-acetylated to some extent by the same enzymes. N-acetyltransferases are cytosolic enzymes which utilize the cofactor acetyl-coenzyme A (acetyl-CoA) to transfer the acetyl group in a two step process. In the first step, the acetyl group is transferred from acetyl-CoA to an active site cysteine residue; in the second step, the acetyl group is transferred to the substrate amino group and the enzyme is regenerated.

In contrast to most other DME classes, there are a limited number of known N-acetyltransferases. In humans, there are two highly similar enzymes, NAT1 and NAT2; mice appear to have a third form of the enzyme, NAT3. The human forms of N-acetyltransferase have independent regulation (NAT1 is widely-expressed, whereas NAT2 is in liver and gut only) and overlapping substrate preferences. Both enzymes appear to accept most substrates to some extent, but NAT1 does prefer some substrates (para-aminobenzoic acid, para-aminosalicylic acid, sulfamethoxazole, and sulfanilamide), while NAT2 prefers others (isoniazid, hydralazine, procainamide, dapsone, aminoglutethimide, and sulfamethazine).

Clinical observations of individuals taking the antituberculosis drug isoniazid in the 1950s led to the description of fast and slow acetylators of the compound. These phenotypes were shown subsequently to be due to mutations in the NAT2 gene which affected enzyme activity or stability. The slow isoniazid acetylator phenotype is very prevalent in Middle Eastern populations (approx. 70%), and is less prevalent in Caucasian (approx. 50%) and Asian (<25%) populations. More recently, functional polymorphism in NAT1 has been detected, with approximately 8% of the population tested showing a slow acetylator phenotype (Butcher, N. J. et al. (1998) Pharmacogenetics 8: 67-72). Since NAT1 can activate some known aromatic amine carcinogens, polymorphism in the widely-expressed NAT1 enzyme may be important in determining cancer risk.

Aminotransferase

Aminotransferases comprise a family of pyridoxal 5′-phosphate (PLP)-dependent enzymes that catalyze transformations of amino acids. Aspartate aminotransferase (AspAT) is the most extensively studied PLP-containing enzyme. It catalyzes the reversible transamination of dicarboxylic L-amino acids, aspartate and glutamate, and the corresponding 2-oxo acids, oxaloacetate and 2-oxoglutarate. Other members of the family included pyruvate aminotransferase, branched-chain amino acid aminotransferase, tyrosine aminotransferase, aromatic aminotransferase, alanine: glyoxylate aminotransferase (AGT), and kynurenine aminotransferase (Vacca, R. A. et al. (1997) J. Biol. Chem.272: 21932-21937).

Primary hyperoxaluria type-1 is an autosomal recessive disorder resulting in a deficiency in the liver-specific peroxisomal enzyme, alanine: glyoxylate aminotransferase-1. The phenotype of the disorder is a deficiency in glyoxylate metabolism. In the absence of AGT, glyoxylate is oxidized to oxalate rather than being transaminated to glycine. The result is the deposition of insoluble calcium oxalate in the kidneys and urinary tract, ultimately causing renal failure (Lumb, M. J. et al. (1999) J. Biol. Chem. 274: 20587-20596).

Kynurenine aminotransferase catalyzes the irreversible transamination of the L-tryptophan metabolite L-kynurenine to form kynurenic acid. The enzyme may also catalyze the reversible transamination reaction between L-2-aminoadipate and 2-oxoglutarate to produce 2-oxoadipate and L-glutamate. Kynurenic acid is a putative modulator of glutamatergic neurotransmission, thus a deficiency in kynurenine aminotransferase may be associated with pleotrophic effects (Buchli, R. et al. (1995) J. Biol. Chem. 270: 29330-29335).

Catechol-o-methyltransferase

Catechol-o-methyltransferase (COMT) catalyzes the transfer of the methyl group of S-adenosylmethionine (AdoMet; SAM) donor to one of the hydroxyl groups of the catechol substrate (e.g., L-dopa, dopamine, or DBA). Methylation of the 3′-hydroxyl group is favored over methylation of the 4′-hydroxyl group and the membrane bound isoform of COMT is more regiospecific than the soluble form. Translation of the soluble form of the enzyme results from utilization of an internal start codon in a full-length mRNA (1.5 kb) or from the translation of a shorter mRNA (1.3 kb), transcribed from an internal promoter. The proposed S_{N}2-like methylation reaction requires Mg²⁺ and is inhibited by Ca²⁺. The binding of the donor and substrate to COMT occurs sequentially. AdoMet first binds COMT in a Mg²⁺-independent manner, followed by the binding of Mg²⁺ and the binding of the catechol substrate.

The amount of COMT in tissues is relatively high compared to the amount of activity normally required, thus inhibition is problematic. Nonetheless, inhibitors have been developed for in vitro use (e.g., galates, tropolone, U-0521, and 3′,4′-dihydroxy-2-methyl-propiophetropolone) and for clinical use (e.g., nitrocatechol-based compounds and tolcapone). Administration of these inhibitors results in the increased half-life of L-dopa and the consequent formation of dopamine. Inhibition of COMT is also likely to increase the half-life of various other catechol-structure compounds, including but not limited to epinephrine/norepinephrine, isoprenaline, rimiterol, dobutamine, fenoldopam, apomorphine, and α-methyldopa. A deficiency in norepinephrine has been linked to clinical depression, hence the use of COMT inhibitors could be useful in the treatment of depression. COMT inhibitors are generally well tolerated with minimal side effects and are ultimately metabolized in the liver with only minor accumulation of metabolites in the body (Männistö, P. T. and Kaakkola, S. (1999) Pharmacological Reviews 51: 593-628).

Copper-zinc Superoxide Dismutases

Copper-zinc superoxide dismutases are compact homodimeric metalloenzymes involved in cellular defenses against oxidative damage. The enzymes contain one atom of zinc and one atom of copper per subunit and catalyze the dismutation of superoxide anions into O₂ and H₂O₂. The rate of dismutation is diffusion-limited and consequently enhanced by the presence of favorable electrostatic interactions between the substrate and enzyme active site. Examples of this class of enzyme have been identified in the cytoplasm of all the eukaryotic cells as well as in the periplasm of several bacterial species. Copper-zinc superoxide dismutases are robust enzymes that are highly resistant to proteolytic digestion and denaturing by urea and SDS. In addition to the compact structure of the enzymes, the presence of the metal ions and intrasubunit disulfide bonds is believed to be responsible for enzyme stability. The enzymes undergo reversible denaturation at temperatures as high as 70° C. (Battistoni, A. et al. (1998) J. Biol. Chem. 273:655-5661).

Overexpression of superoxide dismutase has been implicated in enhancing freezing tolerance of transgenic Alfalfa as well as providing resistance to environmental toxins such as the diphenyl ether herbicide, acifluorfen (McKersie, B. D. et al. (1993) Plant Physiol. 103: 1155-1163). In addition, yeast cells become more resistant to freeze-thaw damage following exposure to hydrogen peroxide which causes the yeast cells to adapt to further peroxide stress by upregulating expression of superoxide dismutases. In this study, mutations to yeast superoxide dismutase genes had a more detrimental effect on freeze-thaw resistance than mutations which affected the regulation of glutathione metabolism, long suspected of being important in determining an organism's survival through the process of cryopreservation (Jong-In Park, J-I. et al. (1998) J. Biol. Chem. 273: 22921-22928).

Expression of superoxide dismutase is also associated with Mycobacterium tuberculosis, the organism that causes tuberculosis. Superoxide dismutase is one of the ten major proteins secreted by M. tuberculosis and its expression is upregulated approximately 5-fold in response to oxidative stress. M. tuberculosis expresses almost two orders of magnitude more superoxide dismutase than the nonpathogenic mycobacterium M. smegmatis, and secretes a much higher proportion of the expressed enzyme. The result is the secretion of 350-fold more enzyme by M. tuberculosis than M. smegmatis, providing substantial resistance to oxidative stress (Harth, G. and Horwitz, M. A. (1999) J. Biol. Chem. 274: 4281-4292).

The reduced expression of copper-zinc superoxide dismutases, as well as other enzymes with anti-oxidant capabilities, has been implicated in the early stages of cancer. The expression of copper-zinc superoxide dismutases has been shown to be lower in prostatic intraepithelial neoplasia and prostate carcinomas, compared to normal prostate tissue (Bostwick, D. G. (2000) Cancer 89: 123-134).

Phosphodiesterases

Phosphodiesterases make up a class of enzymes which catalyze the hydrolysis of one of the two ester bonds in a phosphodiester compound. Phosphodiesterases are therefore crucial to a variety of cellular processes. Phosphodiesterases include DNA and RNA endonucleases and exonucleases, which are essential for cell growth and replication, and topoisomerases, which break and rejoin nucleic acid strands during topological rearrangement of DNA. A Tyr-DNA phosphodiesterase functions in DNA repair by hydrolyzing dead-end covalent intermediates formed between topoisomerase I and DNA (Pouliot, J. J. et al. (1999) Science 286: 552-555; Yang, S.-W. (1996) Proc. Natl. Acad. Sci. USA 93: 11534-11539).

Acid sphingomyelinase is a phosphodiesterase which hydrolyzes the membrane phospholipid sphingomyelin to produce ceramide and phosphorylcholine. Phosphorylcholine is used in the synthesis of phosphatidylcholine, which is involved in numerous intracellular signaling pathways, while ceramide is an essential precursor for the generation of gangliosides, membrane lipids found in high concentration in neural tissue. Defective acid sphingomyelinase leads to a build-up of sphingomyelin molecules in lysosomes, resulting in Niemann-Pick disease (Schuchman, E. H. and S. R. Miranda (1997) Genet. Test. 1: 13-19).

Glycerophosphoryl diester phosphodiesterase (also known as glycerophosphodiester phosphodiesterase) is a phosphodiesterase which hydrolyzes deacetylated phospholipid glycerophosphodiesters to produce sn-glycerol-3-phosphate and an alcohol. Glycerophosphocholine, glycerophosphoethanolamine, glycerophosphoglycerol, and glycerophosphoinositol are examples of substrates for glycerophosphoryl diester phosphodiesterases. A glycerophosphoryl diester phosphodiesterase from E. coli has broad specificity for glycerophosphodiester substrates (Larson, T. J. et al. (1983) J. Biol. Chem. 248: 5428-5432).

Cyclic nucleotide phosphodiesterases (PDEs) are crucial enzymes in the regulation of the cyclic nucleotides cAMP and cGMP. cAMP and cGMP function as intracellular second messengers to transduce a variety of extracellular signals including hormones, light, and neurotransmitters. PDEs degrade cyclic nucleotides to their corresponding monophosphates, thereby regulating the intracellular concentrations of cyclic nucleotides and their effects on signal transduction. Due to their roles as regulators of signal transduction, PDEs have been extensively studied as chemotherapeutic targets (Perry, M. J. and G. A. Higgs (1998) Curr. Opin. Chem. Biol. 2: 472-81; Torphy, J. T. (1998) Am. J. Resp. Crit. CareMed. 157: 351-370).

Families of mammalian PDEs have been classified based on their substrate specificity and affinity, sensitivity to cofactors, and sensitivity to inhibitory agents (Beavo, J. A. (1995) Physiol. Rev. 75: 725-748; Conti, M. et al. (1995) Endocrine Rev. 16: 370-389). Several of these families contain distinct genes, many of which are expressed in different tissues as splice variants. Within PDE families, there are multiple isozymes and multiple splice variants of these isozymes (Conti, M. and S. L. C. Jin (1999) Prog. Nucleic Acid Res. Mol. Biol. 63: 1-38). The existence of multiple PDE families, isozymes, and splice variants is an indication of the variety and complexity of the regulatory pathways involving cyclic nucleotides (Houslay, M. D. and G. Milligan (1997) Trends Biochem. Sci. 22: 217224).

Type 1 PDEs (PDE1s) are Ca²⁺/calmodulin-dependent and appear to be encoded by at least three different genes, each having at least two different splice variants (Kakkar, R. et al. (1999) Cell Mol. Life Sci. 55: 1164-1186). PDE1s have been found in the lung, heart, and brain. Some PDE1 isozymes are regulated in vitro by phosphorylation/dephosphorylation. Phosphorylation of these PDE1 isozymes decreases the affinity of the enzyme for calmodulin, decreases PDE activity, and increases steady state levels of cAMP (Kakkar, supra). PDE1s may provide useful therapeutic targets for disorders of the central nervous system, and the cardiovascular and immune systems due to the involvement of PDE1s in both cyclic nucleotide and calcium signaling (Perry, M. J. and G. A. Higgs (1998) Curr. Opin. Chem. Biol. 2: 472-481).

PDE2s are cGMP-stimulated PDEs that have been found in the cerebellum, neocortex, heart, kidney, lung, pulmonary artery, and skeletal muscle (Sadhu, K. et al. (1999) J. Histochem. Cytochem. 47: 895-906). PDE2s are thought to mediate the effects of cAMP on catecholamine secretion, participate in the regulation of aldosterone (Beavo, supra), and play a role in olfactory signal transduction (Juilfs, D. M. et al. (1997) Proc. Natl. Acad. Sci. USA 94: 3388-3395). PDE3s have high affinity for both cGMP and cAMP, and so these cyclic nucleotides act as competitive substrates for PDE3s. PDE3s play roles in stimulating myocardial contractility, inhibiting platelet aggregation, relaxing vascular and airway smooth muscle, inhibiting proliferation of T-lymphocytes and cultured vascular smooth muscle cells, and regulating catecholamine-induced release of free fatty acids from adipose tissue. The PDE3 family of phosphodiesterases are sensitive to specific inhibitors such as cilostamide, enoximone, and lixazinone. Isozymes of PDE3 can be regulated by cAMP-dependent protein kinase, or by insulin-dependent kinases (Degerman, E. et al. (1997) J. Biol. Chem. 272: 6823-6826).

PDE4s are specific for cAMP, are localized to airway smooth muscle, the vascular endothelium, and all inflammatory cells; and can be activated by cAMP-dependent phosphorylation. Since elevation of cAMP levels can lead to suppression of inflammatory cell activation and to relaxation of bronchial smooth muscle, PDE4s have been studied extensively as possible targets for novel anti-inflammatory agents, with special emphasis placed on the discovery of asthma treatments. PDE4 inhibitors are currently undergoing clinical trials as treatments for asthma, chronic obstructive pulmonary disease, and atopic eczema. All four known isozymes of PDE4 are susceptible to the inhibitor rolipram, a compound which has been shown to improve behavioral memory in mice (Barad, M. et al. (1998) Proc. Natl. Acad. Sci. USA 95: 15020-15025). PDE4 inhibitors have also been studied as possible therapeutic agents against acute lung injury, endotoxemia, rheumatoid arthritis, multiple sclerosis, and various neurological and gastrointestinal indications (Doherty, A. M. (1999) Curr. Opin. Chem. Biol. 3: 466-473).

PDE5 is highly selective for cGMP as a substrate (Turko, I. V. et al. (1998) Biochemistry 37: 4200-4205), and has two allosteric cGMP-specific binding sites (McAllister-Lucas, L. M. et al. (1995) J. Biol. Chem. 270: 30671-30679). Binding of cGMP to these allosteric binding sites seems to be important for phosphorylation of PDE5 by cGMP-dependent protein kinase rather than for direct regulation of catalytic activity. High levels of PDE5 are found in vascular smooth muscle, platelets, lung, and kidney. The inhibitor zaprinast is effective against PDE5 and PDEls. Modification of zaprinast to provide specificity against PDE5 has resulted in sildenafil (VIAGRA; Pfizer, Inc., New York N.Y.), a treatment for male erectile dysfunction (Terrett, N. et al. (1996) Bioorg. Med. Chem. Lett. 6: 1819-1824). Inhibitors of PDE5 are currently being studied as agents for cardiovascular therapy (Perry, M. J. and G. A. Higgs (1998) Curr. Opin. Chem. Biol. 2: 472-481).

PDE6s, the photoreceptor cyclic nucleotide phosphodiesterases, are crucial components of the phototransduction cascade. In association with the G-protein transducin, PDE6s hydrolyze cGMP to regulate cGMP-gated cation channels in photoreceptor membranes. In addition to the cGMP-binding active site, PDE6s also have two high-affinity cGMP-binding sites which are thought to play a regulatory role in PDE6 function (Artemyev, N. O. et al. (1998) Methods 14: 93-104). Defects in PDE6s have been associated with retinal disease. Retinal degeneration in the rd mouse (Yan, W. et al. (1998) Invest. Opthalmol. Vis. Sci. 39: 2529-2536), autosomal recessive retinitis pigmentosa in humans (Danciger, M. et al. (1995) Genomics 30: 1-7), and rod/cone dysplasia 1 in Irish Setter dogs (Suber, M. L. et al. (1993) Proc. Natl. Acad. Sci. USA 90: 3968-972) have been attributed to mutations in the PDE6B gene.

The PDE7 family of PDEs consists of only one known member having multiple splice variants (Bloom, T. J. and J. A. Beavo (1996) Proc. Natl. Acad. Sci. USA 93: 14188-14192). PDE7s are cAMP specific, but little else is known about their physiological function. Although mRNAs encoding PDE7s are found in skeletal muscle, heart, brain, lung, kidney, and pancreas, expression of PDE7 proteins is restricted to specific tissue types (Han, P. et al. (1997) J. Biol. Chem. 272: 16152-16157; Perry, M. J. and G. A. Higgs (1998) Curr. Opin. Chem. Biol. 2: 472-481). PDE7s are very closely related to the PDE4 family; however, PDE7s are not inhibited by rolipram, a specific inhibitor of PDE4s (Beavo, supra).

PDE8s are cAMP specific, and are closely related to the PDE4 family. PDE8s are expressed in thyroid gland, testis, eye, liver, skeletal muscle, heart, kidney, ovary, and brain. The cAMP hydrolyzing activity of PDE8s is not inhibited by the PDE inhibitors rolipram, vinpocetine, milrinone, IBMX (3-isobutyl-1-methylxanthine), or zaprinast, but PDE8s are inhibited by dipyridamole (Fisher, D. A. et al. (1998) Biochem. Biophys. Res. Commun. 246: 570-577; Hayashi, M. et al. (1998) Biochem. Biophys. Res. Commun. 250: 751-756; Soderling, S. H. et al. 1998) Proc. Natl. Acad. Sci. USA 95: 8991-8996).

PDE9s are CGMP specific and most closely resemble the PDE8 family of PDEs. PDE9s are expressed in kidney, liver, lung, brain, spleen, and small intestine. PDE9s are not inhibited by sildenafil (VIAGRA; Pfizer, Inc., New York NY), rolipram, vinpocetine, dipyridamole, or IBMX (3-isobutyl-lmethylxanthine), but they are sensitive to the PDE5 inhibitor zaprinast (Fisher, D. A. et al. (1998) J. Biol. Chem. 273: 15559-15564; Soderling, S. H. et al. (1998) J. Biol. Chem. 273: 15553-15558).

PDE10s are dual-substrate PDEs, hydrolyzing both cAMP and cGMP. PDE10s are expressed in brain, thyroid, and testis. (Soderling, S. H. et al. (1999) Proc. Natl. Acad. Sci. USA 96: 7071-7076; Fujishige, K. et al. (1999) J. Biol. Chem. 274: 18438-18445; Loughney, K. et al. (1999) Gene 234: 109117).

PDEs are composed of a catalytic domain of about 270-300 amino acids, an N-terminal regulatory domain responsible for binding cofactors, and, in some cases, a hydrophilic C-terminal domain of unknown function (Conti, M. and S.-L. C. Jin (1999) Prog. Nucleic Acid Res. Mol. Biol. 63: 1-38). A conserved, putative zinc-binding motif, HDXXHXGXXN, has been identified in the catalytic domain of all PDEs. N-terminal regulatory domains include non-catalytic cGMP-binding domains in PDE2s, PDE5s, and PDE6s; calmodulin-binding domains in PDE1s; and domains containing phosphorylation sites in PDE3s and PDE4s. In PDE5, the N-terminal cGMP-binding domain spans about 380 amino acid residues and comprises tandem repeats of the conserved sequence motif N (R/K) XnFX3DE (McAllister-Lucas, L. M. et al. (1993) J. Biol. Chem. 268: 22863-22873). The NKXnD motif has been shown by mutagenesis to be important for cGMP binding (Turko, I. V. et al. (1996) J. Biol. Chem. 271: 22240-22244). PDE families display approximately 30% amino acid identity within the catalytic domain; however, isozymes within the same family typically display about 85-95% identity in this region (e.g. PDE4A vs PDE4B). Furthermore, within a family there is extensive similarity (>60%) outside the catalytic domain; while across families, there is little or no sequence similarity outside this domain.

Many of the constituent functions of immune and inflammatory responses are inhibited by agents that increase intracellular levels of cAMP (Verghese, M. W. et al. (1995) Mol. Pharmacol. 47: 1164-1171). A variety of diseases have been attributed to increased PDE activity and associated with decreased levels of cyclic nucleotides. For example, a form of diabetes insipidus in mice has been associated with increased PDE4 activity, an increase in low-Kₘ cAMP PDE activity has been reported in leukocytes of atopic individuals, and PDE3 has been associated with cardiac disease.

Many inhibitors of PDEs have been identified and have undergone clinical evaluation (Perry, M. J. and G. A. Higgs (1998) Curr. Opin. Chem. Biol. 2: 472-481; Torphy, T. J. (1998) Am. J. Respir. Crit. Care Med. 157: 351-370). PDE3 inhibitors are being developed as antithrombotic agents, antihypertensive agents, and as cardiotonic agents useful in the treatment of congestive heart failure. Rolipram, a PDE4 inhibitor, has been used in the treatment of depression, and other inhibitors of PDE4 are undergoing evaluation as anti-inflammatory agents. Rolipram has also been shown to inhibit lipopolysaccharide (LPS) induced TNF-a, which has been shown to enhance HIV-1 replication in vitro. Therefore, rolipram may inhibit HIV-1 replication (Angel, J. B. et al. (1995) AIDS 9: 1137-1144). Additionally, rolipram, based on its ability to suppress the production of cytokines such as TNF-a and b and interferon g, has been shown to be effective in the treatment of encephalomyelitis. Rolipram may also be effective in treating tardive dyskinesia and was effective in treating multiple sclerosis in an experimental animal model (Sommer, N. et al. (1995) Nat. Med. 1: 244-248; Sasaki, H. et al. (1995) Eur. J. Pharmacol. 282: 71-76).

Theophylline is a nonspecific PDE inhibitor used in the treatment of bronchial asthma and other respiratory diseases. Theophylline is believed to act on airway smooth muscle function and in an anti-inflammatory or immunomodulatory capacity in the treatment of respiratory diseases (Banner, K. H. and C. P. Page (1995) Eur. Respir. J. 8: 996-1000). Pentoxifylline is another nonspecific PDE inhibitor used in the treatment of intermittent claudication and diabetes-induced peripheral vascular disease. Pentoxifylline is also known to block TNF-a production and may inhibit HIV-1 replication (Angel et al., supra).

PDEs have been reported to affect cellular proliferation of a variety of cell types (Conti et al. (1995) Endocrine Rev. 16: 370-389) and have been implicated in various cancers. Growth of prostate carcinoma cell lines DU145 and LNCaP was inhibited by delivery of cAMP derivatives and PDE inhibitors (Bang, Y. J. et al. (1994) Proc. Natl. Acad. Sci. USA 91: 5330-5334). These cells also showed a permanent conversion in phenotype from epithelial to neuronal morphology. It has also been suggested that PDE inhibitors have the potential to regulate mesangial cell proliferation (Matousovic, K. et al. (1995) J. Clin. Invest. 96: 401-410) and lymphocyte proliferation (Joulain, C. et al. (1995) J. Lipid Mediat. Cell Signal. 11: 63-79). A cancer treatment has been described that involves intracellular delivery of PDEs to particular cellular compartments of tumors, resulting in cell death (Deonarain, M. P. and A. A. Epenetos (1994) Br. J. Cancer 70: 786-794).

Phosphotriesterases

Phosphotriesterases (PTE, paraoxonases) are enzymes that hydrolyze toxic organophosphorus compounds and have been isolated from a variety of tissues. The enzymes appear to be lacking in birds and insects, but is abundant in mammals, explaining the reduced tolerance of birds and insects to organophosphorus compound (Vilanova, E. and Sogorb, M. A. (1999) Crit. Rev. Toxicol. 29: 21-57). Phosphotriesterases play a central role in the detoxification of insecticides by mammals. Phosphotriesterase activity varies among individuals and is lower in infants than adults. Knockout mice are markedly more sensitive to the organophosphate-based toxins diazoxon and chlorpyrifos oxon (Furlong, C. E., et al. (2000) Neurotoxicology 21: 91-100). PTEs have attracted interest as enzymes capable of the detoxification of organophosphate-containing chemical waste and warfare reagents (e.g., parathion), in addition to pesticides and insecticides. Some studies have also implicated phosphotriesterase in atherosclerosis and diseases involving lipoprotein metabolism.

Thioesterases

Two soluble thioesterases involved in fatty acid biosynthesis have been isolated from mammalian tissues, one which is active only toward long-chain fatty-acyl thioesters and one which is active toward thioester with a wide range of fatty-acyl chain-lengths. These thioesterases catalyze the chain-terminating step in the de novo biosynthesis of fatty acids. Chain termination involves the hydrolysis of the thioester bond which links the fatty acyl chain to the 4′-phosphopantetheine prosthetic group of the acyl carrier protein (ACP) subunit of the fatty acid synthase (Smith, S. (1981a) Methods Enzymol. 71: 181-188; Smith, S. (1981b) Methods Enzymol. 71: 188-200).

E. coli contains two soluble thioesterases, thioesterase I (TEI) which is active only toward long-chain acyl thioesters, and thioesterase II (TEII) which has a broad chain-length specificity (Naggert, J. et al. (1991) J. Biol. Chem. 266: 11044-11050). E. coli TEII does not exhibit sequence similarity with either of the two types of mammalian thioesterases which function as chain-terminating enzymes in de novo fatty acid biosynthesis. Unlike the mammalian thioesterases, E. coli TEII lacks the characteristic serine active site gly-X-ser-X-gly sequence motif and is not inactivated by the serine modifying agent diisopropyl fluorophosphate. However, modification of histidine 58 by iodoacetamide and diethylpyrocarbonate abolished TEII activity. Overexpression of TEII did not alter fatty acid content in E. coli, which suggests that it does not function as a chain-terminating enzyme in fatty acid biosynthesis (Naggert et al., supra). For that reason, Naggert et al. (supra) proposed that the physiological substrates for E. coli TEII may be coenzyme A (CoA)-fatty acid esters instead of ACP-phosphopanthetheine-fatty acid esters.

Carboxlesterases

Mammalian carboxylesterases constitute a multigene family expressed in a variety of tissues and cell types. Isozymes have significant sequence homology and are classified primarily on the basis of amino acid sequence. Acetylcholinesterase, butyrylcholinesterase, and carboxylesterase are grouped into the serine super family of esterases (B-esterases). Other carboxylesterases included thyroglobulin, thrombin, Factor IX, gliotactin, and plasminogen. Carboxylesterases catalyze the hydrolysis of ester and amide-groups from molecules and are involved in detoxification of drugs, environmental toxins, and carcinogens. Substrates for carboxylesterases include short-and long-chain acyl-glycerols, acylcarnitine, carbonates, dipivefrin hydrochloride, cocaine, salicylates, capsaicin, palmitoyl-coenzyme A, imidapril, haloperidol, pyrrolizidine alkaloids, steroids, p-nitrophenyl acetate, malathion, butanilicaine, and isocarboxazide. The enzymes often demonstrate low substrate specificity. Carboxylesterases are also important for the conversion of prodrugs to their respective free acids, which may be the active form of the drug (e.g., lovastatin, used to lower blood cholesterol) (reviewed in Satoh, T. and Hosokawa, M. (1998) Annu. Rev. Pharmacol. Toxicol. 38: 257-288).

Neuroligins are a class of molecules that (i) have N-terminal signal sequences, (ii) resemble cell-surface receptors, (iii) contain carboxylesterase domains, (iv) are highly expressed in the brain, and (v) bind to neurexins in a calcium-dependent manner. Despite the homology to carboxylesterases, neuroligins lack the active site serine residue, implying a role in substrate binding rather than catalysis (Ichtchenko, K. et al. (1996) J. Biol. Chem. 271: 2676-2682).

Squalene Epoxidase

Squalene epoxidase (squalene monooxygenase, SE) is a microsomal membrane-bound, FAD-dependent oxidoreductase that catalyzes the first oxygenation step in the sterol biosynthetic pathway of eukaryotic cells. Cholesterol is an essential structural component of cytoplasmic membranes acquired via the LDL receptor-mediated pathway or the biosynthetic pathway. In the latter case, all 27 carbon atoms in the cholesterol molecule are derived from acetyl-CoA (Stryer, L., supra). SE converts squalene to 2, 3 (S)-oxidosqualene, which is then converted to lanosterol and then cholesterol. The steps involved in cholesterol biosynthesis are summarized below (Stryer, L (1988) Biochemistry. W. H Freeman and Co., Inc. New York. pp. 554-560 and Sakakibara, J. et al. (1995) 270: 17-20):

acetate (from Acetyl-CoA)→3-hydoxy-3-methyl-glutaryl CoA→mevalonate→5-phosphomevalonate→5-pyrophosphomevalonate→isopentenyl pyrophosphate→dimethylallyl pyrophosphate→geranyl pyrophosphate→farnesyl pyrophosphate→squalene→squalene epoxide→lanosterol→cholesterol.

While cholesterol is essential for the viability of eukaryotic cells, inordinately high serum cholesterol levels results in the formation of atherosclerotic plaques in the arteries of higher organisms. This deposition of highly insoluble lipid material onto the walls of essential blood vessels (e.g., coronary arteries) results in decreased blood flow and potential necrosis of the tissues deprived of adequate blood flow. HMG-CoA reductase is responsible for the conversion of 3-hydroxyl-3-methylglutaryl CoA (HMG-CoA) to mevalonate, which represents the first committed step in cholesterol biosynthesis. HMG-CoA is the target of a number of pharmaceutical compounds designed to lower plasma cholesterol levels. However, inhibition of MHG-CoA also results in the reduced synthesis of non-sterol intermediates (e.g., mevalonate) required for other biochemical pathways. SE catalyzes a rate-limiting reaction that occurs later in the sterol synthesis pathway and cholesterol is the only end product of the pathway following the step catalyzed by SE. As a result, SE is the ideal target for the design of anti-hyperlipidemic drugs that do not cause a reduction in other necessary intermediates (Nakamura, Y. et al. (1996) 271: 8053-8056).

Epoxide Hydrolases

Epoxide hydrolases catalyze the addition of water to epoxide-containing compounds, thereby hydrolyzing epoxides to their corresponding 1,2-diols. They are related to bacterial haloalkane dehalogenases and show sequence similarity to other members of the α/β hydrolase fold family of enzymes (e.g., bromoperoxidase A2 from Streptomyces aureofaciens, hydroxymuconic semialdehyde hydrolases from Pseudomonas putida, and haloalkane dehalogenase from Xanthobacter autotrophicus). Epoxide hydrolases are ubiquitous in nature and have been found in mammals, invertebrates, plants, fungi, and bacteria. This family of enzymes is important for the detoxification of xenobiotic epoxide compounds which are often highly electrophilic and destructive when introduced into an organism. Examples of epoxide hydrolase reactions include the hydrolysis of cis-9,10-epoxyoctadec-9 (Z)-enoic acid (leukotoxin) to form its corresponding diol, threo-9,10-dihydroxyowtadec-12 (Z)-enoic acid (leukotoxin diol), and the hydrolysis of cis-12,13-epoxyoctadec-9 (Z)-enoic acid (isoleukotoxin) to form its corresponding diol threo-12, 13-dihydroxyoctadec-9 (Z)-enoic acid (isoleukotoxin diol). Leukotoxins alter membrane permeability and ion transport and cause inflammatory responses. In addition, epoxide carcinogens are known to be produced by cytochrome P450 as intermediates in the detoxification of drugs and environmental toxins.

The enzymes possess a catalytic triad composed of Asp (the nucleophile), Asp (the histidine-supporting acid), and His (the water-activating histidine). The reaction mechanism of epoxide hydrolase proceeds via a covalently bound ester intermediate initiated by the nucleophilic attack of one of the Asp residues on the primary carbon atom of the epoxide ring of the target molecule, leading to a covalently bound ester intermediate (Michael Arand, M. et al. (1996) J. Biol. Chem. 271: 4223-4229; Rink, R. et al. (1997) J. Biol. Chem. 272: 14650-14657; Argiriadi, M. A. et al. (2000) J. Biol. Chem. 275: 15265-15270).

Enzymes Involved In Tyrosine Catalysis

The degradation of the amino acid tyrosine to either succinate and pyruvate or fumarate and acetoacetate, requires a large number of enzymes and generates a large number of intermediate compounds. In addition, many xenobiotic compounds may be metabolized using one or more reactions that are part of the tyrosine catabolic pathway. While the pathway has been studied primarily in bacteria, tyrosine degradation is known to occur in a variety of organisms and is likely to involve many of the same biological reactions.

The enzymes involved in the degradation of tyrosine to succinate and pyruvate (e.g., in Artlirobacter species) include 4-hydroxyphenylpyruvate oxidase, 4-hydroxyphenylacetate 3-hydroxylase, 3,4-dihydroxyphenylacetate 2,3-dioxygenase, 5-carboxymethyl-2-hydroxymuconic semialdehyde dehydrogenase, trans, cis-5-carboxymethyl-2-hydroxymuconate isomerase, homoprotocatechuate isomerase/decarboxylase, cis-2-oxohept-3-ene-1,7-dioate hydratase, 2,4-dihydroxyhepttrans-2-ene-1,7-dioate aldolase, and succinic semialdehyde dehydrogenase.

The enzymes involved in the degradation of tyrosine to fumarate and acetoacetate (e.g., in Pseudontonas species) include 4-hydroxyphenylpyruvate dioxygenase, homogentisate 1,2-dioxygenase, maleylacetoacetate isomerase, and fumarylacetoacetase. 4-hydroxyphenylacetate 1-hydroxylase may also be involved if intermediates from the succinate/pyruvate pathway are accepted.

Additional enzymes associated with tyrosine metabolism in different organisms include 4-chlorophenylacelate-3,4-dioxygenase, aromatic aminotransferase, 5-oxopent-3-ene-1,2,5-tricarboxylate decarboxylase, 2-oxo-hept-3-ene-1,7-dioate hydratase, and 5-carboxymethyl-2-hydroxymuconate isomerase (Ellis, L. B. M. et al. (1999) Nucleic Acids Res. 27: 373-376; Wackett, L. P. and Ellis, L. B. M. (1996) J. Microbiol. Meth. 25: 91-93; and Schmidt, M. (1996) Amer. Soc. Microbiol. News 62: 102).

In humans, acquired or inherited genetic defects in enzymes of the tyrosine degradation pathway may result in hereditary tyrosinemia. One form of this disease, hereditary tyrosinemia 1 (HT1) is caused by a deficiency in the enzyme fumarylacetoacetate hydrolase, the last enzyme in the pathway in organisms that metabolize tyrosine to fumarate and acetoacetate. HT1 is characterized by progressive liver damage beginning at infancy, and increased risk for liver cancer (Endo, F. et al. (1997) J. Biol. Chem. 272: 24426-24432).

An enzyme of one system can act on several drugs and drug metabolites. The rate of metabolism of a drug differs between individuals and between ethnic groups, owing to the existence of enzymatic polymorphism within each system. Metabolic phenotypes have been generally characterized as poor metabolizers (PM), extensive metabolizers (EM), and ultra-extensive metabolizers (UEM). Knowledge of a metabolic phenotype is clinically useful for the following reasons:

1) a phenotype may be correlated to an individual's susceptibility to toxic chemicals, diseases and cancers;

2) a phenotype may provide a physician with valuable information for quickly determining a safe and therapeutically-effective drug treatment regimen for an individual; and

3) individual phenotypes may provide valuable rationales for the design of therapeutic drugs.

To date, the ability to characterize multiple phenotypic determinants for the purpose of identifying individual phenotypes, drug treatment compatibility and susceptibility has been limited by the complexities of multiple metabolic pathways, and the lack of efficient and effective procedures for making these determinations. Currently, the determination of an individual's phenotype for a given metabolic enzyme can be performed either via direct metabolic phenotyping or indirect extrapolation of an individual's genotype to a given phenotype.

Direct phenotyping involves the use a probe substrate known to be metabolized by a given enzyme. The rate of metabolism of the probe substrate is measured and this rate of metabolism is used to determine a metabolic phenotype. Although labor intensive and costly procedures for direct phenotyping have been known for many years these procedures are not readily adaptable for a clinical environment, nor are they practical for measuring multiple phenotypic determinants. For example, enzymatic phenotypes may be determined by measurements of the molar (or chiral) ratio of metabolites of a drug or a probe substrate in a urine sample from a individual by high-pressure liquid chromatography (HPLC), capillary electrophoresis (CE) or stereo-selective capillary gas chromatography. These determination methods are time-consuming, onerous, and employ systems and equipment that are not readily available in a clinical laboratory. Methodologies for the rapid determination of multiple determinants of a metabolic phenotypic are not available, and as a result, valuable information concerning an individual's phenotype is not considered on a routine basis in a clinical environment.

Indirect phenotyping can be defined as assigning a phenotype based on non-functional measurements. These non-functional measurements include genotyping, haplotyping, gene expression and protein expression analysis. The patent application, WO 00/63683 provides an extensive description of various methods developed to perform the aforementioned analysis.

Genotyping is performed by analyzing the genetic sequence of a gene coding for a specific enzyme by a polymerase chain reaction assay (PCR) or a PCR with a restriction fragment length polymorphism assay (PCR-RFLP). The gene is examined for the presence of genetic mutations that can be linked to increased or decreased enzyme levels or activity, which in turn result in a specific phenotype, i.e. a slow metabolizer vs. a fast metabolizer. The genotype is a theoretical measurement of what an individual's phenotype should be. Haplotyping is an extension of genotyping in which the genotype of different gene alleles are considered. For example if a person had one wild type (wt) gene sequence and one mutant (mt) gene sequence, the individual would have a wt/mt haplotype. Gene expression and protein expression analysis is defined as the measurement of mRNA/cDNA and protein levels respectively.

Indirect phenotyping may be limited by several factors that can result in an alteration in the theoretical phenotype. For example it has been well established that genotype does not always correlate with phenotype, likewise gene expression does not always correlate with protein expression, and protein expression does not always correlate with protein function. Indirect phenotyping fails to account for many factors that affect protein function including but not limited to post-translational protein modification, polypharmacy, and exposure to inducers or inhibitors. Furthermore, other limitations include the potential complexity of performing a complete genotyping. The mutation sequence must first be identified before they can be examined in a genotyping assay. Subsequent to identification, the mutation must be linked to a definitive effect on phenotype. For some enzymes, there appear to be very few mutations and those found have been well characterized, while for other enzymes multiple mutations are present with new mutations being found regularly (e.g. CYP2D6 has over 53 mutations and 48 allelic variants). Therefore, while genotyping for CYP2C19 might be performed with relatively few measurements, a complete and accurate genotyping of CYP2D6 would be complex and require multiple measurements.

Indirect phenotyping suffers from complexity and the direct phenotyping techniques are not easily accessible to clinical settings,

Physicians routinely prescribe treatment regimes without knowledge of an individual's metabolic capability (phenotype) or genotype for metabolism. Accordingly, a trial and error treatment regime is initiated, often at the expense of severe side effects and loss of valuable treatment time.

The need for a method to predict an individual's response to a drug therapy (both efficacy of therapy and occurrence of side effects) has been recognized by many in the field. The importance of drug metabolizing can be explained as follows. If inhibition of a particular system leads to toxicity, then low gene or protein expression of components of this system might be used to identify individuals with high risk of toxicity. Likewise those individual's with high expression levels would be considered to be at low risk. However, if the individual classified as a low risk individual, also has low metabolism of the drug, then the drug will remain in the system much longer and may have the time to eliminate the function of the system which as a result leads to toxicity. Conversely, if an individual has low system activity but is also a rapid drug metabolizer, than it is possible that there will not be sufficient drug present at any given point to induce toxicity by inhibiting the system. Therefore, the knowledge of an individual's drug metabolizing capabilities is an essential component of individualized drug therapy.

The ability to rapidly and accurately identify multiple metabolic phenotypic determinants on an individual basis would provide a physician with valuable individual-specific information that could be readily applied in selecting a safe and effective treatment regime for that individual. Similarly, knowledge of multi-determinant metabolic phenotypics would also find valuable application in research and drug development. In particular, individual phenotypes could be identified prior to a drug treatment trial. Moreover, knowledge of multi-determinant metabolic phenotypes would have applications in the development of new drugs, so-called rational drug design.

### SUMMARY OF THE INVENTION

One aim of the present invention is to provide a method for selecting an individual treatment regime.

Accordingly, another aim of the present invention is to provide a method for the individualization of treatment with an Alzheimer's disease agent.

Yet another aim of the present invention is to provide a method for selecting candidates for clinical treatment trials.

Still another aim of the present invention is to provide a method of using multi-determinant phenotyping for the individualization of treatment with an Alzheimer's disease agent.

In accordance with one aspect of the present invention, there is provided a method of characterizing a multi-determinant metabolic phenotype for at least one Alzheimer's disease agent, wherein a plurality of phenotypic determinants are identified as corresponding to respective metabolic characteristics; said method comprising: a) administering to an individual a probe substrate specific to metabolic pathway(s) for said at least one Alzheimer's disease agent; b) detecting metabolites of said metabolic pathway(s) in a biological sample from said individual in response to said probe substrate; and c) characterizing respective phenotypic determinants of said multi-determinant metabolic phenotype based on detected metabolites.

In accordance with yet another aspect of the present invention, there is provided a method of using a multi-determinant metabolic phenotype to individualize a treatment regimen for at least one Alzheimer's disease agent for an individual, wherein the multi-determinant metabolic phenotype of said individual is determined; a safe and therapeutically effective dose of said at least one Alzheimer's disease agent treatment is determined and/or selected based on said multi-determinant metabolic phenotype of said individual.

In accordance with yet a further aspect of the invention, there is provided a method of administering to an individual at least one Alzheimer's disease agent, said method comprising: a) determining a multi-determinant metabolic phenotype of said individual; and administering a safe and therapeutically effective dose of said at least one Alzheimer's disease agent to said individual, wherein said dose has been determined based on a metabolic profile of said individual corresponding to said individual's metabolic phenotype for said at least one Alzheimer's disease agent as represented by said multi-determinant metabolic phenotype.

In accordance with still a further aspect of the invention, there is provided an assay system for detecting the presence of enzyme-specific metabolites in a biological sample, said sample obtained from an individual treated with a known amount of at least one probe substrate for at least one Alzheimer's disease agent, specific for metabolic pathways of said metabolites, said assay comprising: a) means for receiving said biological sample, including a plurality of affinity complexation agents contained therein; b) means for detecting presence of said enzyme-specific metabolites bound to said affinity complexation agents; and c) means for quantifying ratios of said metabolites to provide corresponding phenotypic determinants; wherein said phenotypic determinants provide a metabolic phenotypic profile of said individual.

In accordance with yet another aspect of the present invention there is provided a method of using an enzyme-specific assay for the individualization of treatment with at least one Alzheimer's disease agent, said method comprising: a) conducting said assay to identify phenotypic determinants in a biological sample obtained from an individual treated with a probe substrate for said at least one Alzheimer's disease agent; b) determining a rate of drug metabolism according to said determinants; and c) determining and/or selecting a safe and therapeutically effective dose of said class of Alzheimer's disease agent for said individual based on said rate.

In accordance with yet another aspect of the present invention there is provided a method of screening a plurality of individuals for participation in a drug treatment trial assessing the therapeutic effect of at least one Alzheimer's disease agent, said method comprising: selecting individuals having a metabolic phenotype characterized as effective for metabolizing said at least one Alzheimer's disease agent.

In accordance with yet another aspect of the present invention there is provided a method of screening a plurality of individuals for treatment with at least one Alzheimer's disease agent, said method comprising: a) genotyping said individuals to identify individuals lacking at least one allelic variation known to prompt toxicity of said at least one Alzheimer's disease agent; and b) selecting individuals having a metabolic phenotype characterized as effective for metabolizing said at least one Alzheimer's disease agent.

In accordance with yet another aspect of the present invention there is provided a method of screening a plurality of individuals for participation in a drug treatment trial assessing the therapeutic effect of a candidate Alzheimer's disease agent treatment, said method comprising: a) genotyping each of said individuals to identify individuals lacking at least one allelic variation known to prompt the toxicity of said Alzheimer's disease agent; and b) characterizing a multi-determinant metabolic phenotype of said identified individuals of step a) to determine each individual's ability to metabolize said Alzheimer's disease agent.

For the purpose of the present invention the following terms are defined below.

The term “phenotypic determinant” is intended to mean a qualitative or quantitative indicator of an enzyme-specific capacity of an individual.

The term “individualization” as it appears herein with respect to therapy is intended to mean a therapy having specificity to at least an individual's phenotype as calculated according to a predetermined formula on an individual basis.

The term “biological sample” is intended to mean a sample obtained from a biological entity and includes, but is not to be limited to, any one of the following: tissue, cerebrospinal fluid, plasma, serum, saliva, blood, nasal mucosa, urine, synovial fluid, microcapillary microdialysis and breath.

The term “Alzheimer's disease agent” is intended to mean an agent(s) and/or medicine(s) used to treat the symptoms of Alzheimer's disease, particularly the loss of cognitive functions.

The term “treatment” is intended to mean any administration of a pharmaceutical compound to an individual to treat, cure, alleviate, improve, diminish or inhibit a condition in the individual, including, without limitation, administering Alzheimer's disease agent for delaying the loss of cognitive function in an individual.

The term “individual treated” is intended to mean any individual being subjected to the administration of i) a pharmaceutical compound, for treating, curing, alleviating, improving, diminishing or inhibiting a condition, or ii) a probe substrate for determining multi-determinant metabolic phenotype.

### BRIEF DESCRIPTION OF THE DRAWINGS

FIG. 1 illustrates metabolites of the CYP3A4 enzymatic pathway according to an embodiment of the present invention;

FIG. 2 illustrates metabolites of the NAT2 enzymatic pathway according to an embodiment of the present invention;

FIG. 3 illustrates metabolites of the CYP1A2 enzymatic pathway according to another embodiment of the present invention;

FIG. 4 illustrates metabolites of the NAT1 enzymatic pathway according to another embodiment of the present invention;

FIG. 5 illustrates metabolites of the CYP2A6 enzymatic pathway according to another embodiment of the present invention;

FIG. 6 illustrates metabolites of the CYP2C19 enzymatic pathway according to another embodiment of the present invention;

FIG. 7 illustrates metabolites of the CYP2C9 enzymatic pathway according to another embodiment of the present invention;

FIG. 8 illustrates metabolites of the CYP2D6 enzymatic pathway according to another embodiment of the present invention;

FIG. 9 illustrates metabolites of the CYP2E1 enzymatic pathway according to another embodiment of the present invention;

FIG. 10 illustrates the scheme of the general immunosensor design depicting the intimate integration of immunological recognition at the solid-state surface and the signal transduction;

FIG. 11 illustrates the principle of SPR technology;

FIG. 12 illustrates a TSM immunosensor device;

FIG. 13 illustrates the synthetic routes for the production of AAMU and 1X derivatives used in accordance with one embodiment of the present invention;

FIGS. 14 to 17 show other AAMU and 1X derivatives which can be used for raising antibodies in accordance with another embodiment of the present invention;

FIG. 18 illustrates the absorbance competitive antigen ELISA curves of AAMU-Ab and 1X-Ab in accordance with one embodiment of the present invention;

FIG. 19 is a histogram of molar ratio of AAMU/1X;

FIG. 20 illustrates an ELISA array in accordance with an embodiment of the present invention;

FIG. 21 illustrates an ELISA array in accordance with another embodiment of the present invention;

FIG. 22 illustrates an ELISA detection system in accordance with another embodiment of the present invention.

FIG. 23 illustrates an assay system in accordance with another embodiment of the present invention; and

FIG. 24 illustrates individualized dosing schemes for direct vs. indirect phenotyping in accordance with yet another embodiment of the present invention.

### DETAILED DESCRIPTION OF THE INVENTION

The present invention relates to the individualization of drug treatment. In particular, the present invention relates to the individualization of drug treatment with Alzheimer's disease agents. Based on a phenotypic characterization of an individual's capacity to metabolize cytochrome P450-specific Alzheimer's disease agent, the present invention provides a system and method for determining a dosage of an Alzheimer's disease agent on an individual basis. Currently, only three agents have been improved for the therapy of Alzheimer's disease (tacrine, donezepil, and galantamine). All three agents are metabolized by the CYP450. Using donezepil as an example, the present invention provides a method for quickly and accurately determining phenotypic determinants for at least one Alzheimer's disease agent-specific enzyme and/or metabolic pathway that can be used to characterize an individual's CYP3A4 specific phenotype. In doing so, a characterization of an individual's ability to metabolize an Alzheimer's disease agent can be made and a corresponding drug dosage specific for that individual can be determined.

Further, the present invention provides a method for determining multiple phenotypic determinants that can be used to characterize a phenotypic profile of an individual that will exemplify that individual's ability to metabolize a given drug or group of drugs. Although most drugs are metabolized by a primary enzymatic pathway, such as CYP3A4 metabolizes donezepil, it is often the case that a given drug may be metabolized by multiple enzymes. As a result, it may be preferred to characterize an individual's phenotypic profile for a plurality of metabolic enzymes prior to selecting a corresponding drug treatment regime. Knowledge of an individual's metabolic phenotype may be applied clinically in determining a phenotype-specific drug dosage based on the individual's capacity to metabolize the drug. Other factors representing an individual's capacity to metabolize a drug may also find application in the present invention, together with a phenotypic profile for obtaining individualization of therapy.

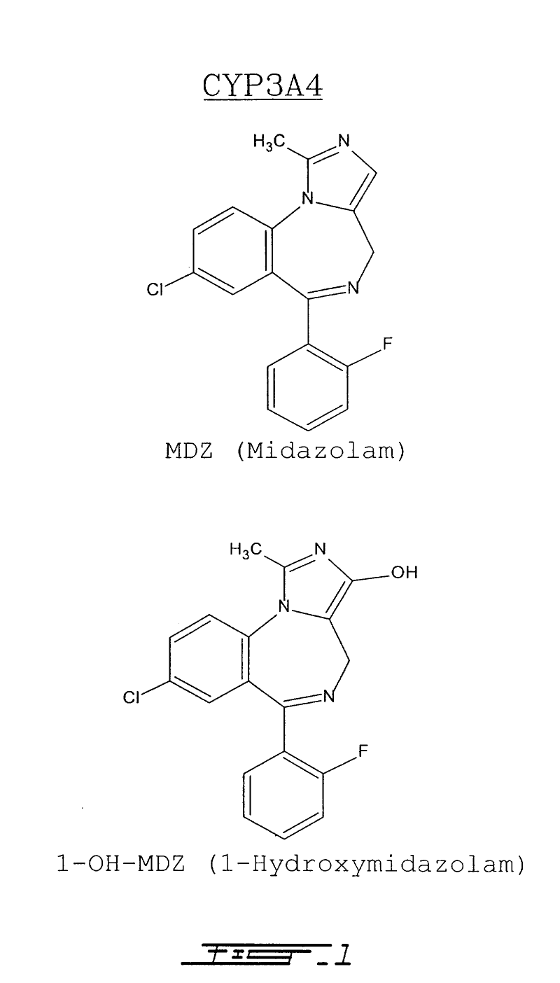

Accordingly, a system of the present invention is exemplified in accordance with a protocol for determining phenotypic determinants for NAT2. This protocol is adapted to provide a system for determining phenotypic determinants for at least CYP3A4 or at least CYP2D6 in accordance with the present invention. The determination of metabolic determinants for CYP3A4 or CYP2D6 may be performed as a single determination or in combination with methods of determining a phenotypic profile for at least one of the following enzymes: NAT1, NAT2, CYP1A2, CYP2A6, CYP2D6, CYP2E1, CYP3A4, CYP2C9 and CYP2C19, the metabolites of which are illustrated in FIGS. 1-9. These enzymes are involved in the metabolism of a large number of drugs, and as a result have important implications in the outcome of individual drug treatment regimes, and hence, clinical trial studies. These enzymes and their corresponding phenotypic determinants as described herein are provided as a representative example of determinants for the purposes of exemplifying the multi-determinant metabolic phenotyping of the present invention. However, the present invention is not limited thereto.

The present invention provides the ability to identify multiple phenotypic determinants of these enzymatic pathways for use in the individualization of drug treatment with Alzheimer's disease agent.

Alzheimer's Disease

Alzheimer's disease is one of the most common chronic conditions of older persons. Overall, it affects about 1 in 10 Americans older than the age of 65. Occurrence of the disease is strikingly related to age, affecting about 15% of those between the ages of 75 and 84 and about half of those older than the age of 85. Between 4 and 5 million people in the United States currently have the disease. Because the number of people in the United States older than 65 is the most rapidly growing segment of the population, the number of people with the disease is projected to triple by the middle of the 21^{st} century, making Alzheimer's disease a public health problem of truly staggering proportions.

Clinical Presentation and Course Early Stages

Most individuals are mildly forgetful and complain of such memory deficits as forgetting names or where they placed household items. The individual may seem concerned but has no social or employment problems and shows no evidence of memory deficit during a clinical interview. The next stage is characterized by decreased performance in demanding work or social situations. Individuals complain of poor concentration, difficulty in remembering words and names, more prominent memory loss, and confusion, and they may report that co-workers have noticed their relatively poor performance. Some individuals present initially with visual-spatial deficits, whereas others may have difficulty with speech early in the course of the illness. Later, individuals are easily disoriented and get lost when traveling to an unfamiliar location. Anxiety and depression are common, and many individuals begin to deny symptoms. Changes in personality and judgment can begin to cause problems with family and co-workers.

Intermediate Stages

As the illness progresses, individuals become unable to travel alone and are unable to handle their personal finances. Memory for recent events is drastically impaired, and individuals display a decreased knowledge of current events. Complex tasks are impossible, but individuals remain fairly well oriented to time and person and can travel to very familiar places, like the corner drugstore. Many individuals are aware of their deficits and are capable of understanding what is happening to them. Individuals instinctively withdraw from previously challenging situations and may even have trouble with the activities of daily living. Difficulty with speech is more pronounced. Denial may become pronounced. Anxiety increases, along with suspiciousness and agitation. The most difficult behavior at this point is wandering and pacing, which makes it important that individuals begin to wear an identification bracelet available through the Alzheimer's Association.

Late-stage Disease

Individuals can no longer survive without some assistance. They are unable to recall major relevant aspects of their current lives or even the names of close friends and family members. Delusions and hallucinations are common. For example, the spouse is accused of being an impostor, or individuals talk to imaginary persons or their own reflection in the mirror. Depression, agitation, and violent behavior may occur.

Frequently, individuals are disoriented to time or place. However, they generally remain able to eat and use the toilet without assistance, but they may have difficulty in properly choosing and putting on clothing.

Advanced Disease

In the last stages of the disease, individuals become totally incapacitated and disoriented. They eventually forget their own name and may not recognize their spouse. Incontinence is common, with loss of bladder and bowel control. Personality and emotional changes become very pronounced, although these occasionally occur even in the earliest stages. Eventually, all verbal abilities are lost, motor skills deteriorate, and individuals require total care. Total dependence on the caregiver ensues, and it is important to monitor for caregiver stress, or “burnout.” Generalized cortical and focal neurologic signs and symptoms are frequently present. Death usually occurs from total debilitation or infection.

Clinical Course

The course of Alzheimer's disease from onset to death varies from 2 to 20 years. The average is about 6 to 8 years. Typically, the illness progresses at a fairly constant rate. If it has rapidly developed during the past year, it is likely to continue at that rate. A slowly progressive illness during the past 5 to 10 years suggests that the individual may survive for a number of years, especially if the individual is in otherwise good physical health. Other clinical features found to be independent predictors of more rapid progression to incapacity and death include hallucinations, paranoia, delusions, misidentification syndromes, extrapyramidal signs, and a in low score on initial psychometric testing.

Treatment of Cognitive Function

Dementia typically evolves over many years. For much of this time, the individual is not totally disabled and is more functional in a familiar and stable environment. There are many areas in which intervention can improve quality of life for both the individual and the caregiver. Although the disease is defined by its effect on cognitive function, affective and behavioral disturbances and motor signs may be the targets of intervention. Finally, there are interventions aimed at altering the course of the disease, including reducing disability, institutionalization, caregiver stress, and death.

Acetylcholine is the neurotransmitter of several central nervous system neuronal circuits. One circuit projects from the basal forebrain to the hippocampus and parts of the cerebral cortex. Agents that augment this system have been under investigation for the treatment of Alzheimer's disease for more than 20 years. The investigations have followed four basic strategies: (1) administering a precursor of acetylcholine synthesis, (2) stimulating acetylcholine release, (3) inhibiting the enzyme that catalyzes acetylcholine degradation, and (4) mimicking the action of acetylcholine at the cholinergic receptor. Of these, inhibition of acetylcholinesterase rapidly hydrolyzes acetylcholine to choline and acetic acid and has met with the most success. Inhibiting acetylcholinesterase prolongs the action of acetylcholine in the synapse, allowing for more effective transmission. Three acetylcholinesterase inhibitors, tacrine (Cognex), donepezil (Aricept), and galantine hydrobromide (Reminyl) have been approved by the Food and Drug Administration (FDA) for the symptomatic treatment of Alzheimer's disease. Several others, including apan, CI-1017, CP-118,954, DAD2000, DHEA, Exelon TDS, gama secretase Inhibitor, M2 Antagonist, marinol, memantine, metrifonate, NDD 094A, NGD 97-1, nicotinic compounds, NXY-059, pakio, phenserine, physostigmine, promem, rasagiline mesylate, and rivastigmine have been submitted to the FDA or are under investigation and may be approved in the future.

Tacrine

Tacrine (Cognex) was the first acetylcholinesterase inhibitor to be approved for the treatment of Alzheimer's disease. With a duration of action of about 6 hours, the drug must be given four times daily. Several double-blind, placebo-controlled studies have documented a small, but significant, improvement in cognitive function, about 2 points on the ADAS-Cog over 12 weeks, with 40 mg four times daily for 12 to 30 weeks. Unfortunately, up to half of the persons on tacrine in these studies had to discontinue the drug due to side effects, including asymptomatic hepatotoxicity, nausea, vomiting, and diarrhea. Because of the relatively high frequency of hepatotoxicity, the manufacturer's recommendation is to slowly increase the dose of tacrine, checking serum transaminase levels weekly. For individuals with mild to moderate Alzheimer's disease, the dosage should start at 10 mg four times a day and then increased to 20 mg four times a day, then to 30 mg four times a day, and finally to 40 mg four times a day. Increases should take place at 6-week intervals. Thus, it takes about 4 months to achieve a stable dose.

The drug should be discontinued if transaminase levels rise above three times the upper limit of normal. Because of the limited benefit, the risk of side effects, and the difficulty of use, tacrine has already been supplanted by newer agents.

Donezepil

Donepezil (Aricept) was the second acetylcholinesterase inhibitor to be approved for the treatment of Alzheimer's disease. Donepezil has several advantages over tacrine, including a longer half-life and fewer side effects. Because of its longer half-life, the drug can be administered once per day. In double-blind, placebo-controlled studies, donepezil has been administered at doses of either 5 mg or 10 mg per day for 12 to 24 weeks. About 85% of subjects taking 5 mg, and 70% of subjects taking 10 mg completed the studies. Side effects, including nausea, vomiting, and diarrhea, were mild and limited. There were no significant elevations of serum transaminase levels. Both dosages of 5 mg per day and 10 mg per day were superior to placebo, with an effect size similar to that of tacrine—about 3 points on the ADAS-Cog over 24 weeks. Although the 10-mg dose was not clearly better than the 5-mg dose, the higher dose is recommended because the response to cholinesterase inhibitors, in general, tends to be dose dependent. For individuals with mild to moderate Alzheimer's disease, the dosage should start at 5 mg per day for 1 month and then increased to 10 mg per day if the 5-mg dose is tolerated.

Galantaamine Hydrobromide

Galantamine Hydrobromide (Reminyl) is the third drug approved for the treatment of Alzheimer's disease. As with tacrine and donezepil, galantamine directly inhibits acetylcholinesterase (AchE) activity, while demonstrating much weaker activity on butyrylcholinesterase (BuChE). Galantamine also stimulates pre- and postsynaptic nicotinic receptors, although the clinical significance of this finding is yet unclear. Numerous variants and analogues of galantamine have also been developed, with varying potency in inhibiting AChE activity. GAL is readily absorbed after oral administration, with a t(max) of 52 min and a plasma elimination t(1/2) of 5.7 h. The efficacy of GAL administered to Alzheimer's disease (AD) individuals has been well demonstrated by large-scale clinical trials. Typical of AChEIs, the most common adverse events associated with GAL are nausea and vomiting. Evidence to date suggests galanthamine to be similar to other AChEIs in improving cognitive function in AD individuals.

In half of individuals with mild to moderate forms of Alzheimer's who usually worsen over time, cognitive scores and daily functions were maintained at or above baseline throughout one year of treatment. In trials of 3 to 6 months duration, recipients of galantamine (16 to 24 mg/day) achieved significant improvements in cognitive and global symptoms relative to placebo recipients. Galantamine also improved activities of daily living in these individuals and significantly reduced the requirement for caregiver assistance with activities of daily living. The effect of the drug will vary for different people. Some will not notice an effect at all. Others may find that condition improves, or that they stay the same when they would have expected to get gradually less able because of the progression of the disease.

The overall effect size of tacrine, donepezil and galantamine is small, which makes it difficult to clearly determine their efficacy on an individual basis. Consensus regarding recommendations for discontinuation of these drugs has been elusive. On one hand, because the trials were aimed at detecting improvement of cognitive function, it has been advocated that if improvement cannot be documented in 24 to 30 weeks, the drug should be discontinued. In contrast, because there are few alternatives, others recommend that in the absence of significant side effects, the drugs should be continued until the subject becomes severely impaired. This debate is likely to continue until the discovery of agents with robust and reproducible effects that can clearly be determined on an individual-by-individual basis or the demonstration that a particular agent actually slows deterioration.

Ergoloid Mesylates

Ergoloid mesylates (Hydergine) has been available in the United States for the treatment of a variety of conditions, including dementia, for decades and is extensively used worldwide. Its mechanism of action is unclear, but it appears to effect several central nervous system receptor systems. Numerous trials of Hydergine have been conducted. Overall, there may be a small benefit of the drug at doses of up to 7.5 mg per day, although the results of the larger and more rigorous double-blind, placebo-controlled clinical trials have been negative.

Ginko Extracts

Extracts of Ginkgo biloba are among the most commonly prescribed drugs in Europe and are used for a variety of conditions, including progressive degenerative dementia.

Four Gingko preparations have been used in clinical trials, differing only by the amount of ginkgo flavone glycosides. Several studies have reported small benefits of the Gingko biloba extract Egb 761 for Alzheimer's disease. High rates of dropout and loss to follow-up in the largest and most recent double-blind, placebo-controlled study, however, preclude a definitive conclusion regarding the efficacy of the Gingko biloba. The lack of clear efficacy, combined with the small magnitude of the effect and the potential for side effects either alone or when used with commonly used anticoagulants such as aspirin, suggests that it is premature to recommend it to individuals with Alzheimer's disease.

CYP3A4 plays a major role in the metabolism of the Alzheimer's disease agent donezepil. CYP3A4 is therefore considered to be a key factor in an individual's capacity to metabolize donezepil. In accordance with an embodiment of the present invention, a system and method of determining at least an individual's CYP3A4-specific phenotype for use in the individualization of therapy with Alzheimer's disease agents is presented. Other enzymes are also known to be involved in the metabolism of Alzheimer's disease agents, such as CYP1A2, for example. As such, the present invention is not intended to be limited to any one enzyme but provides a means for determining phenotypic determinants of any enzyme known to influence the metabolism of an Alzheimer's disease agent.

In addition, the present invention may further include the use of indirect phenotyping to identify individuals with a particular genotype, which is associated with extremely high risks of toxicity from a particular Alzheimer's disease agent. According to one embodiment of the present invention, those individuals without the “high risk” genotype will be phenotyped and dosed according to their individual molar ratio, while the high risk individuals will not be recommended for treatment with that particular Alzheimer's disease agent. By employing genotyping in combination with phenotyping to screen individuals for treatment with Alzheimer's disease agent, those individuals found to be carrier of a high risk genotype can be eliminated as candidates for such treatment without the necessity of phenotyping.

The integration of phenotyping tests into the drug development process provides for a decreased number of individuals participating in a drug treatment testing trial, as individual screening using phenotyping can be conducted prior to the trial to select those individuals displaying the capability to metabolize the drug of interest safely and effectively. In particular, those individuals identified as being metabolically incompatible with the drug treatment trial can be screened out before undergoing treatment with the drug. This aspect of the present invention provides a means to selectively treat only those individuals identified as having the ability to safely metabolize the drug. In addition, the decrease in individual number will result in decreased costs and allow the drug to reach the market faster. In addition, the clinical use of a phenotypic screening method of the present invention provides the ability to individualize treatments according to phenotypic profiles. In particular, dose specific determinations corresponding to a calculated rate of metabolism for that drug phenotype is possible on an individual basis.

Pre-trial screening would involve the phenotyping of all individuals prior to inclusion in the trial. The phenotype status could then be used to identify those individuals at high risk for serious adverse events (SAE's) and ensure that they were not included in the trial. The remaining individuals would then be treated with drug doses customized in correlation to their level of CYP3A4 activity, in the case of Alzheimer's disease agent. The customized dose would ensure that the individuals were receiving a safe efficacious treatment, corresponding to their ability to safely metabolize the drug. Similarly, according to the present invention, individualized treatment has application in the clinical environment where drug treatment dosages will be customized according to an individual's phenotypic profile or calculated rate of metabolism.

According to the present invention, phenotypic determinants for one or more of the following enzymes may be characterized to provide a phenotypic profile on an individual basis:

CYP3A4

The CYP 3A family constitutes approximately 25% of the total CYP 450 enzymes in the human liver.

Polymorphism

A large degree of inter-individual variability in the expression of the CYP3A4 isoenzymes has been shown in the human liver (>20 fold). However, the activity of CYP3A4 metabolism is distributed unimodally and as a result, there are currently no categorical classifications for distinct subsets of this population. Further, there is currently no evidence of a common allelic variant in the coding region of the gene. Recently, a rare allelic variant was identified in exon 7 (CYP3A4*2). Limited data suggested that this mutation may result in altered substrate dependent kinetics compared with the wt CYP3A gene. It has been considered that the large inter-individual variability in the activity of CYP3A may reflect differences in transcriptional regulation. Another allelic variant in the 5′-flanking region of CYP3A has been identified (CYP3A4*1B) that involves an A→G transition at position −290 from the transcriptional initiation site. It has been speculated that this nucleotide substitution may be associated with a reduced level of CYP3A activity. Ongoing studies are investigating the existence of a common allelic variant linked to CYP3A4 activity.

CYP3A4 metabolizes several drugs and dietary constituents including delavirdine, indinavir, ritonavir, saquinavir, amprenavir, zidovidine (AZT), nelfinavir mesylate, efavirenz, nevirapine, imiquimod, resiquimod, donezepil, lovastatin, simvastatin, pravastatin, fluvastatin, atorvastatin, cerivastatin, rosuvastatin, benzafibrate, clofibrate, fenofibrate, gemfibrozil, niacin, benzodiazepines, erythromycin, dextromethorphan dihydropyridines, cyclosporine, lidocaine, midazolam, nifedipine, and terfenadine.

In addition, CYP3A4 activates environmental pro-carcinogens especially N′-nitrosonornicotine (NNN), 4-methylnitrosamino-1-(3-pyridyl-1-butanone) (NNK), 5-Methylchrysene, and 4,4′-methylene-bis(2-chloroaniline) (tobacco smoke products).

Induction and Inhibition

CYP3A4 is induced by a number of drugs including dexamethasone, phenobarbital, primidone and the antibiotic rifampicin. Conversely, CYP3A4 is inhibited by erythromycin, grapefruit juice, indinavir, ketoconazole, miconazole, quinine, and saquinavir.

Inter Ethnic Differences

Several studies have suggested that the activity of CYP3A4 varies between populations. Plasma levels of a CYP3A4 substrate drug after oral administration were reported to be twofold to threefold higher in Japanese, Mexican, Southeast Asian and Nigerian Populations compared with white persons residing in various countries. In addition, the CYP3A4*1B allele has been reported to be more frequent in African-American populations as compared to European Americans or Chinese populations (66.7% vs. 4.2% vs. 0%, respectively). The rare CYP3A4*2 allele was found in 2.7% of a white population and was absent in the black and Chinese individuals. It is reasonable that, in drug metabolism studies, each ethnic group can be studied separately for evidence of polymorphism and its antimode should not be extrapolated from one ethnic population to another.

Due to the variability in CYP3A4 activity within the population it would be advantageous to be provided with a system and method for quickly and easily determining an individual's CYP3A4 metabolic phenotype prior to administering a CYP3A4-dependant treatment thereto. In particular, such a system and method are believed to have enormous benefit in the individualization of therapy, and in particular with respect to the individualization of therapy with many hyperlipidia agents, including HMG-CoA reductase inhibitors (statins), fibrates, bile acid sequestrants and nicotinic acid (niacin).

Cyclosporine

An example of the need for phenotyping in drug dosing is the case of cyclosporine in the treatment of organ transplant individuals. Cyclosporine is an immunosuppressant (drug) administered post transplant to protect the new organ from being rejected. Plasma levels of this drug are critical as high levels lead to renal toxicity but low levels can lead to organ rejection. Cyclosporine is metabolized via the CYP3A4 system. Several studies have indicated the importance of monitoring CYP3A4 activity in maintaining an effective and safe cyclosporine dose. For these reasons, the utility of a reliable phenotyping test for CYP3A4 is evident.

Direct Phenotypic Determinants of CYP3A4

Different probe substrates can be used to determine the CYP3A4 phenotype (dapsone, testosterone, nifedipine, midazolam, erythromycin, dextromethorphan, cortisol). In accordance with the present invention, suitable probe substrates include without limitation, midazolam, dextromethorphan, erythromycin, dapsone, testosterone, nifedipine and cortisol.

Of these midazolam is the preferred probe substrate. The structures of midazolam and its hydroxylated metabolite, 1′-hydroxymidazolam are illustrated in FIG. 1. In accordance with the present invention, the molar ratio of midazolam and its metabolite is used to determine the CYP3A4 phenotype of the individual as follows:

$$\frac{{1}^{′} \text{-hydroxymidazolam}}{midazolam}$$

An individual's ratio will be considered as indicative of CYP3A4 enzyme activity with a lower ratio indicating poorer metabolism and a higher ratio indicating more extensive metabolism. The activity of CYP3A4 metabolism is distributed unimodally and hence no antimode is present. The levels of CYP3A4 activity as determined by direct phenotyping will be used.

Indirect Phenotypic Determinants of CYP3A4 (Genotyping)

To date only two mutant alleles have been identified for the CYP3A4 gene (CYP3A4*1B and CYP3A4*2). Studies have been unable to correlate these mutations with the large inter-individual variation in CYP3A4 activity. Despite confirmation in this regard to date, the use of indirect phenotyping is contemplated in accordance with the present invention. Ongoing studies continue to investigate this aspect of the present invention.

NAT2

Polymorphism

Individuals are genetically polymorphic in their rate of N-acetylation of drugs via the N-acetyltransferase (NAT2) pathway (Meyer, U.A. (1994) Proc. Natl. Acad. Sci. USA, 91:1983-1984). Two major metabolic phenotypes can be distinguished: fast and slow N-acetylators. Drugs that are individual to N-acetylation polymorphism include sulfonamides (sulfamethazine), antidepressants (phenelzine), antiarrhymics (procainamide), and antihypertensives (hydrazine). Some adverse therapeutic consequences of the acetylator phenotype are peripheral neuropathy and hepatitis. In an opposite manner, the N-acetylation of procainamide produces a therapeutically active metabolite with reduced toxicity. N-acetylation polymorphism has also been linked to the detoxification pathway of some environmental carcinogenic arylamines and there is a higher frequency of bladder cancers among chemical dye workers who are slow N-acetylators.

The NAT2 gene is polymorphic, there have been 9 mutations detected and 14 mutant alleles. Six mutant alleles are responsible for 99% of Caucasian slow acetylators (NAT2*5A, NAT2*5B, NAT2*5C, NAT2*6A, NAT2*7B, and NAT2*13). The NAT2*4 allele is the wild-type allele.

Inter Ethnic Differences

The frequencies of PM (poor metabolizer) and EM (extensive metabolizers) (autosomal recessive trait) show considerable inter ethnic differences for the N-acetylation polymorphism. In Caucasians, the frequencies are approximately 60 and 40%, respectively, while in Orientals, they are 20 and 80%, respectively (Meyer, U.A. (1994) Proc. Natl. Acad. Sci. USA, 91:1983-1984). It is reasonable that, in drug metabolism studies, each ethnic group is studied separately for evidence of polymorphism and its antimode should not be extrapolated from one ethnic population to another.

Direct Phenotyping—Phenotypic Determinants of NAT2

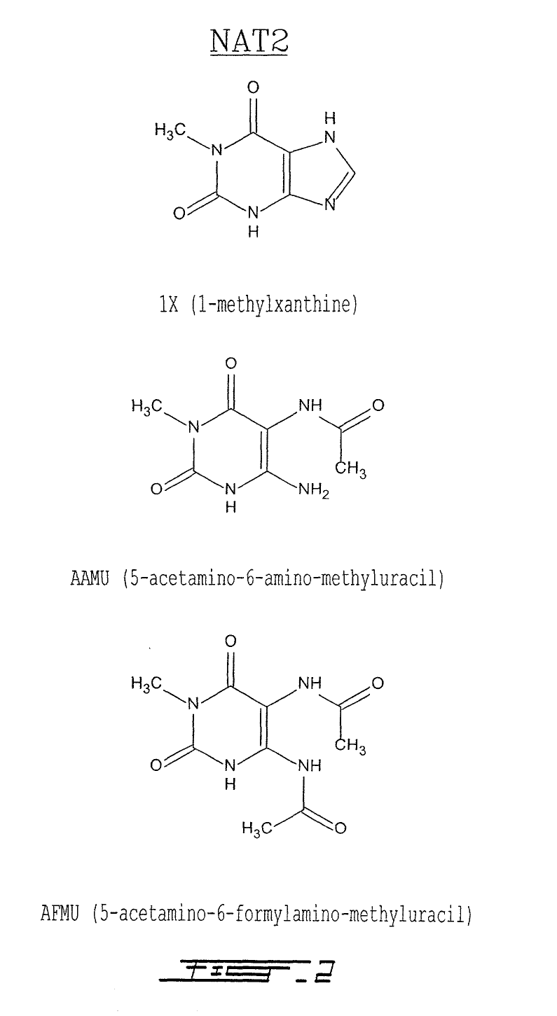

Different probe substrates can be used to determine the NAT2 phenotype. In accordance with the present invention a suitable probe substrate is, without limitation caffeine. Caffeine is widely consumed and relatively safe. A phenotype may be generally determined from ratios of the caffeine metabolites 5-acetamino-6-amino-1-methyluracil (AAMU) or 5-acetamino-6-formylamino-1-methyluracil (AFMU) and 1-methylxanthine (1X) present in urine samples of an individual collected after drinking coffee. The structures of these metabolites are illustrated in FIG. 2. The ratio of these metabolites provides a determination of an individual's N-acetylation (NAT2) phenotype.

AAMU (or AFMU)/1×

In accordance with the present invention, the molar ratio of caffeine metabolites is used to determine the acetylation phenotype of the individual as follows. Individuals with a ratio less than 1.80 are slow acetylators.

Indirect Phenotyping (Genotyping)

An example of NAT2 genotyping involves the amplification of a 547 bp fragment which includes the 5 of the 6 mutant alleles which are responsible for 99% of Caucasian slow acetylators. Analysis of these 5 alleles and the wt allele can be performed by examining 4 mutations (Smith CAD et al. J Med Genet (1997) 34:758-760).

The PCR amplification is performed with the following primers:

| | |
|---|---|
| 5′-GCTGGGTCTGGAAGCTCCTC-3′ | (SEQ ID NO:1) |
| 5′-TTGGGTGATACATACACAAGGG-3′ | (SEQ ID NO:2) |

The analysis of this fragment with 4 restriction digestion enzymes allows the detection of 6 alleles (NAT2*4 (wt) and the mutants NAT2*5A, NAT2*5B, NAT2*5C, NAT2*6 and NAT2*7). Each of the 6 alleles have distinct combinations of the mutations and as each mutation alters a specific restriction digestion enzyme site (KpnI, DdeI, TaqI or BamHI), the performance of 4 separate digestions of the 547 bp fragment will allow the identification of the different alleles.

CYP1A2

CYP1A2 constitutes 15% of the total CYP 450 enzymes in the human liver.

Polymorphism

CYP1A2 may be polymorphic although it remains to be established firmly. To date no mutant alleles have been identified. Three metabolic phenotypes can be distinguished: rapid, intermediate and slow metabolizers. CYP1A2 metabolizes several drugs and dietary constituents including resiquimod, imiquimod, tacrine, acetaminophen, anti pyrine, 17 β-estradiol, caffeine, cloipramine, clozapine, flutamide (antiandrogenic), imipramine, paracetamol, phenacetin, tacrine and theophylline.

In addition, CYP1A2 activates environmental pro-carcinogens, especially heterocyclic amines and aromatic amines. In one study it has been shown that individuals who are fast N-acetylators and have high CYP1A2 activity are at a greater risk for colorectal cancer (35% of cases vs. 16% of controls, OR=2.79 (P=0.00-2).

Induction and Inhibition

CYP1A2 is induced by a number of drugs and environmental factors such as omeprazole, lansoprasole, polyaromatic hydrocarbons and cigarette smoke. CYP1A2 is inhibited by oral contraceptives, ketoconazole, α-napthoflavone, fluvoxamine (serotonine uptake inhibitor), and furafylline.

Inter Ethnic Differences

The activity of CYP1A2 varies broadly (60 to 70 fold) in a given population. Slow, intermediate and rapid CYP1A2 phenotypes have been distinguished. The proportion of these three CYP1A2 phenotypes varied between ethnic groups and countries: % of intermediates: 50, 70, 60, >95, 60, 20 in U.S.A., African-American, China, Japan, Italy and Australia, respectively. It is reasonable that, in drug metabolism studies, each ethnic group can be studied separately for evidence of polymorphism and its antimode should not be extrapolated from one ethnic population to another.

Theophylline

A classical example of the need for phenotyping in drug dosing is the case of theophylline. Theophylline is used in the treatment of asthma. However, theophylline toxicity continues to be a common clinical problem, and involves life-threatening cardiovascular and neurological toxicity. Theophylline is cleared from the body via the CYP1A2 metabolizing system. Inhibition of CYP1A2 by quinolone antibiotic agents or serotonine reuptake inhibitors may result in theophylline toxicity. For these reasons, the utility of a reliable phenotyping test for CYP1A2 is evident.

Direct Phenotypic Determinants of CYP1A2

Different probe substrates can be used to determine the CYP1A2 phenotype (caffeine, theophylline). In accordance with the present invention suitable probe substrates include without limitation, caffeine, theophylline or acetaminophen.

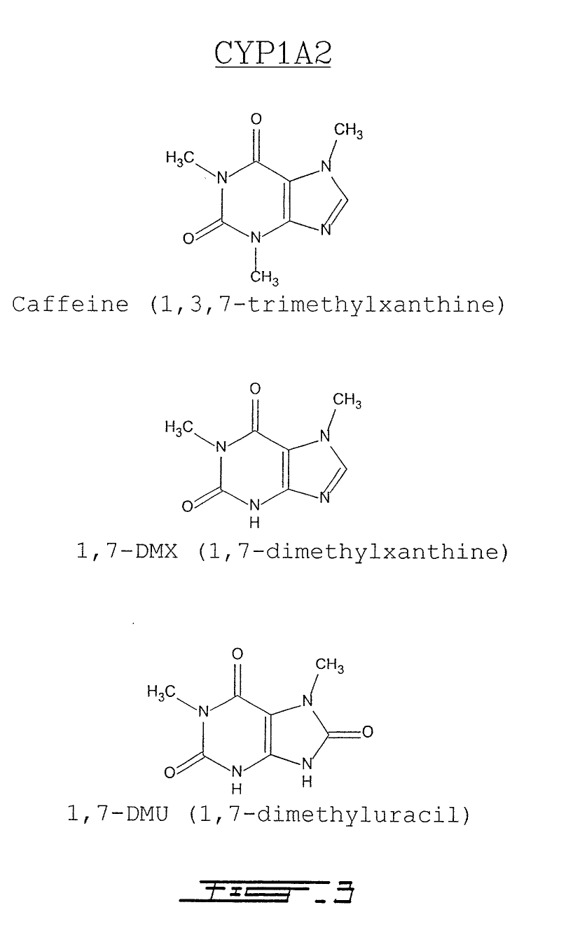

Of these caffeine is the preferred probe substrate. Caffeine is widely consumed and relatively safe. The structure of caffeine and its metabolites 1,7-dimethylxanthine (1,7 DMX) and 1,7-dimethyluric acid (1,7 DMU)are illustrated in FIG. 3.

In accordance with the present invention, the molar ratio of caffeine metabolites is used to determine the CYP1A2 phenotype of the individual as follows:

1,7-dimethylxanthine (1,7 DMX)+1,7-dimethyluric acid (1,7 DMU)/caffeine

Molar ratios of 4 and 12 separate slow, intermediate and fast CYP1A2 metabolizers, respectively (Butler et al. (1992) Pharmacogenetics 2:116-117).

Indirect Phenotypic Determinants of CYP1A2 (Genotyping)

To date no mutant alleles have been identified for the CYP1A2 gene. Therefore, indirect phenotyping is not currently possible for CYP1A2.

NAT1

The NAT1 enzyme catalyzes the N-acetylation of many compounds. It is expressed in the liver as well as in mononuclear leucocytes.

Polymorphism

The NAT1 gene was for a long time classified as monomorphic. However, it is now suggested that NAT1, like the other N-acetyltransferase gene (NAT2), is polymorphic. Studies have demonstrated the presence of one wild type allele (NAT1*4) and six mutant alleles (NAT1*3, NAT1*5, NAT1*10, NAT1*11, NAT1*14 and NAT1*17). NAT1 has two phenotypes: slow and rapid acetylators (e.g. NAT1*4 vs. NAT1*10 genotypes respectively).

NAT1 metabolizes several drugs and dietary constituents including p-aminobenzoic acid, p-aminosalicylic acid, and dapsone.

In addition, NAT1 activates environmental pro-carcinogens, especially diaminobenzidine, N-hydroxy-4-aminobiphenyl, and heterocyclic aromatic amines (MeIQx and PhIP). In one study it has been shown that individuals who have the NAT1*10 allele, and hence are rapid N-acetylators, are at a greater risk for colorectal cancer (OR=1,9; 95% CI=1.2-3.2), while in another study they have an increased risk for bladder cancer (metabolize benzidine).

Inter Ethnic Differences

The activity of NAT1 varies broadly in a given population. Slow, and rapid NAT1 phenotypes have been distinguished. The NAT1*10 genotype that is associated with rapid metabolic phenotype was monitored in three different ethnic populations, Indian, Malaysian and Chinese. The frequency of NAT1*10 allele was 17%, 39% and 30%, respectively. The NAT1*4 genotype, associated with slow metabolizers, had a frequency in the same populations of 50%, 30% and 35%, respectively. Therefore, it is reasonable that, in drug metabolism studies, each ethnic group can be studied separately for evidence of polymorphism and its antimode should not be extrapolated from one ethnic population to another.

Dapsone

A classical example of the need for phenotyping in drug dosing is the case of dapsone. Dapsone is used in the treatment of malaria and is being investigated for the treatment of Pneumocystis carinil pneumonia in AIDS individuals. Adverse effects include rash, anemia, methemoglobinemia, agranulocytosis, and hepatic dysfunction. Dapsone is cleared from the body via the NAT1 metabolizing system. A study has shown a correlation between slow acetylation and increased adverse reactions to dapsone (46% vs. 17% for slow and fast acetylators, respectively). For these reasons, the utility of a reliable phenotyping test is evident.

Phenotypic Determinants of NAT1

Different probe substrates can be used to determine the NAT1 phenotype, such as (p-aminosalicylic acid (pASA), and p-aminobenzoic acid (pABA)). In accordance with the present invention suitable probe substrates include, with out limitation, p-aminosalicylic acid, and p-aminobenzoic acid.

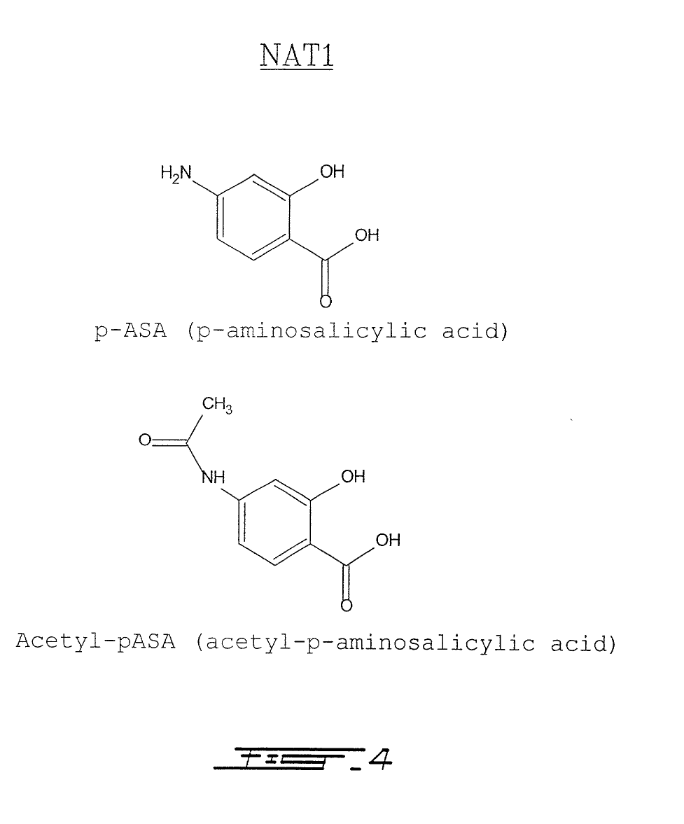

Of these PASA is the preferred probe substrate. The structure of pASA and its acetylated metabolite p-acetylaminosalicylic acid are illustrated in FIG. 4.

In accordance with the present invention, the molar ratio of PASA and its acetylated metabolite is used to determine the NAT1 phenotype of the individual as follows:

pASA

pAcetyl-ASA

Indirect Phenotypic Determinants of NAT1 (Genotyping)

The NAT1 alleles NAT1*4 (wt) and the mutant NAT1*14 can be determined either by PCR-RFLP or allele specific PCR (Hickman, D. et al. (1998); Gut 42:402-409). The PCR-RFLP methodology requires the amplification of the fragment of gene containing the A560G mutation. This is performed with the following primers:

| | |
|---|---|
| 5′-TCCTAGAAGACAGCAACGACC-3′ | (SEQ ID NO:3) |
| 5′-GTGAAGCCCACCAAACAG-3′ | (SEQ ID NO:4) |

This PCR amplification produces a 175 bp fragment that is incubated with the BsaI restriction enzyme. The Nat1*4 allele is cleaved and produces a 155 bp fragment and a 20 bp fragment, while the mutant NAT1*14 is uncleaved.

The NAT1*14 allele is confirmed using an allele specific PCR, with the following primers:

| | |
|---|---|
| 5′-TCCTAGAAGACAGCAACGACC-3′ | (SEQ ID NO:3) |
| 5′-GGCCATCTTTAAAATACATTTT-3′ | (SEQ ID NO:5) |

CYP2A6

CYP2A6 constitutes 4% of the total CYP 450 enzymes in the human liver. CYP2A6 is estimated as participating in 2.5% of drug metabolism.

Polymorphism

CYP2A6 is functionally polymorphic with two mutant alleles, CYP2A6*2 and CYP2A6*3, resulting in an inactive enzyme or the absence of the enzyme, respectively. Two metabolic phenotypes can be distinguished: poor and extensive metabolizers. CYP2A6 metabolizes several drugs including neuroleptic drugs and volatile anesthetics as well as the natural compounds, coumarin, nicotine and aflatoxin B1.

In addition, CYP2A6 activates several components of tobacco smoke (e.g. NNK), as well as 6-aminochrysene. The role of activation of tobacco smoke and the metabolism of nicotine have suggested a role for CYP2A6 in the development of smoking related cancers.

Induction and Inhibition

CYP2A6 is induced by barbiturates, antiepileptic drugs and corticosteroids.

Inter Ethnic Differences

CYP2A6 demonstrates marked inter-individual variability and has demonstrated ethnic related differences. The proportion of the two phenotypes varied between ethnic groups and countries: % of wt genotype (extensive metabolizers): 85, 76, 52, 83, 97.5 in Finnish, English, Japanese, Taiwanese and African-American populations, respectively. It is reasonable that, in drug metabolism studies, each ethnic group can be studied separately for evidence of polymorphism and its antimode should not be extrapolated from one ethnic population to another.

Nicotine

An example of the need for phenotyping in drug dosing is in the delivery of nicotine, for a smoking cessation program. CYP2A6 is the primary means of nicotine metabolism. Extensive CYP2A6 metabolizers will eliminate nicotine at a much higher rate. Identification of individuals with an increased CYP2A6 activity and hence increased nicotine metabolism may identify those individuals that will require higher doses of nicotine at the onset of their attempt to quit smoking with the assistance of a nicotine delivery system. Alternatively, these individuals may benefit from non-nicotine delivery systems for assisting in quitting smoking.

Direct Phenotypic Determinants of CYP2A6

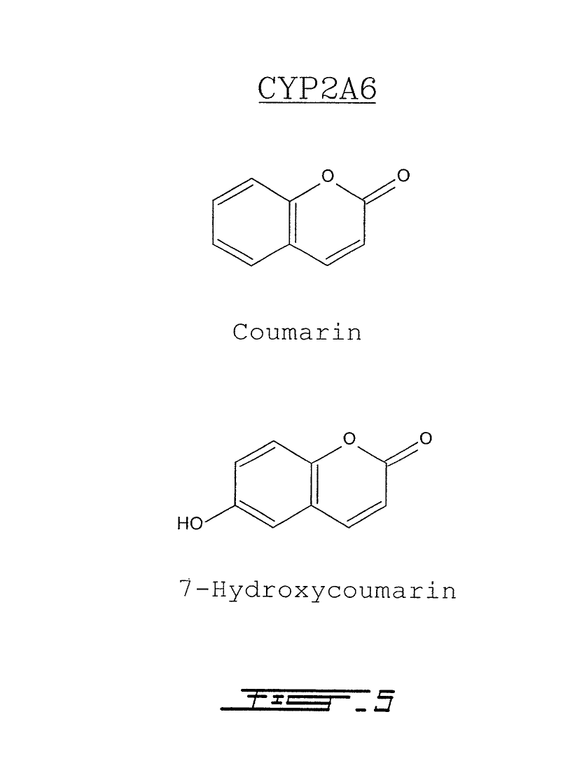

A probe substrate can be used to determine the CYP2A6 phenotype (coumarin). In accordance with the present invention suitable probe substrates include, without limitation, coumarin. The structure of coumarin and its metabolite 7-hydroxycoumarin are illustrated in FIG. 5.

In accordance with the present invention, the molar ratio of coumarin and its metabolite, 7-hydroxycoumarin is used to determine the CYP2A6 phenotype of the individual as follows:

$$\frac{\text{7-hydroxycoumarin}}{coumarin}$$

Indirect Phenotypic Determinants of CYP2A6 (Genotyping)

Currently three alleles have been identified for the CYP2A6 gene, the wild type allele (CYP2A6*1) and two mutant alleles (CYP2A6*2, and CYP2A6*3). The wt allele codes for a fully functional enzyme. The CYP2A6*2 mutant allele codes for an inactive enzyme and the CYP2A6*3 allele does not produce any enzyme.

Determination of an individual genotype can be performed by a combined LA-PCR and PCR-RFLP procedure. In this procedure, specific oligonucleotide primers were used to amplify the CYP2A6/7 gene. The amplified CYP2A6/7 gene is then used as the PCR template to amplify exons 3 and 4 using specific oligonucleotide primers to amplify a 544 bp fragment. This fragment is then digested with the FspI restriction enzyme and a 489 bp fragment re-isolated. This 489 bp fragment is then incubated with both DdeI and XcmI. The digestion patterns were determined by electrophoresis. The wildtype allele produces 330, 87 and 72 bp fragments, the CYP2A6*2 allele yields 189, 141, 87 and 72 bp fragments and the CYP2A6*3 allele yields 270, 87, 72, 60 bp fragments (Nakajima et al. (2000) Clin Pharmacol & Ther. 67(1):57-69).

| | |
|---|---|
| PRIMERS | |
| CYP2AG/7 LA-PCR | |
| 5′-CCTCCCTTGCTGGCTGTGTCCCAAGCTAGGC-3′ | (SEQ ID NO:6) |
| 5′-CGCCCCTTCCTTTCCGCCATCCTGCCCCCAG-3′ | (SEQ ID NO:7) |
| EXON 3/4 PCR | |
| 5′-GCGTGGTATTCAGCAACGGG-3′ | (SEQ ID NO:8) |
| 5′-TGCCCCGTGGAGGTTGACG-3′ | (SEQ ID NO:9) |

CYP2C19

CYP2C19 accounts for about 2% of oxidative drug metabolism. CYP2C19 has been postulated as participating in ˜8% of drug metabolism.

PolyMorphism

Individuals are genetically polymorphic with respect to CYP2C19 metabolism. Two metabolic phenotypes can be distinguished: extensive and poor metabolizers. Two genetic polymorphisms have been identified (CYP2C19*2 and CYP2C19*3) that together explain all of the Oriental poor metabolizers and about 83% of Caucasian poor metabolizers. Both of these mutations introduce stop codons resulting in a truncated and non-functional enzyme.

CYP2C19 metabolizes a variety of compounds including the tricyclic antidepressants amitriptyline, imipramine and clomipramine, the sedatives diazepam and hexobarbital, the gastric proton pump inhibitors, omeprazole, pantoprazole, and lansoprazole, as well as the antiviral nelfinavir mesylate, the antimalarial drug proguanil and the β-blocker propanolol.

Induction and Inhibition

CYP2C19 is inhibited by fluconazole, fluvoxamine, fluoxetine, sertraline, and ritonavir. It is induced by rifampin.

inter Ethnic Differences

The occurrence of the poor metabolizer phenotype for CYP2C19 shows a large inter ethnic variability. Poor metabolizers make up less than 4% of the European and white American populations. While the Korean population has a poor metabolizer frequency of 12.6%, the Chinese 17.4% and the Japanese 22.5%. In addition, the CYP2C19 mutant alleles demonstrate interethnic variability with CYP2C19*2 frequency ranging from 28.9% in the Chinese population to only 13% in European-American population. The CYP2C19*3 allele is absent from the European-American or African-American populations, while occurring at a frequency of 11.7% in both the Korean and Japanese populations.

It is reasonable that, in drug metabolism studies, each ethnic group can be studied separately for evidence of polymorphism and its antimode should not be extrapolated from one ethnic population to another.

Omeprazole

As an example, the benefit of CYP2C19 metabolic phenotyping in drug dosing is evident in the case of omeprazole. Omeprazole is a drug used in the treatment of Heliobacter pylori (H pylori) infections in conjunction with amoxicillin, and is cleared from the body via a CYP2C19 metabolic pathway. Studies have observed higher eradication rates of in CYP2C19 poor metabolizers. Therefore, extensive metabolizers may require higher doses of omeprazole to achieve the same level of H pylori eradication observed in poor metabolizers. For these reasons, the utility of a reliable phenotyping test for CYP2C19 is evident. In particular, an accurate and convenient clinical assay would allow physicians to quickly identify safe and effective treatment regimes for individuals on an individual basis.

Direct Phenotypic Determinants of CYP2C19

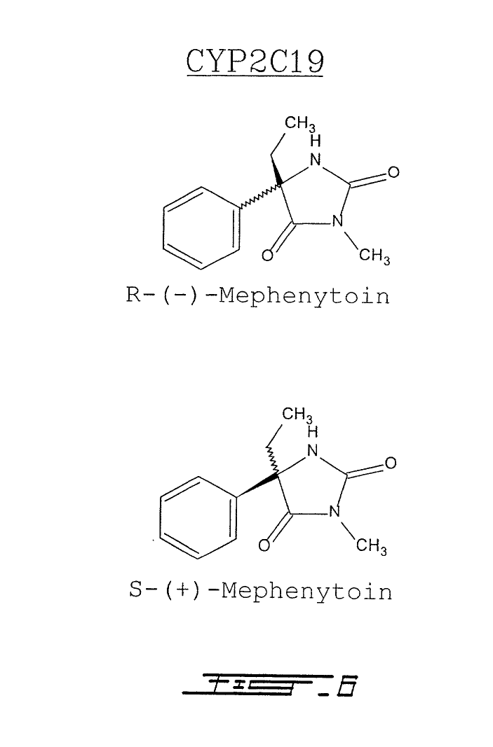

In accordance with an embodiment of the present invention, the ratio of S−(+)mephenytoin and R−(−)mephenytoin in an urine sample may be used to provide a determination of an individual's CYP2C19 phenotype. These metabolites are used as quantitative markers in the determination of a CYP2C19 phenotype on the basis of the use of the preferred probe substrate mephenytoin. However, it is fully contemplated that the present invention is not limited in any respect thereto. The structure of R−(−) and S−(+) mephenytoin and 4-hydroxymephenytoin are illustrated in FIG. 6.

The chiral ratio of S−(+)mephenytoin and R−(−)mephenytoin metabolites, used to determine the CYP2C19 phenotype of the individual, is as follows:

$$\frac{S \text{-} ( + ) Mephenytoin}{R \text{-} ( - ) Mephenytoin}$$

Chiral ratios of close to unity (>0.8) are indicative of fast CYP2C19 metabolizers.

Indirect phenotypic Detreminants of CYP2C19 (Genotyping) As mentioned previously the CYP2C19 has two predominant variant alleles, which account for all Japanese poor metabolizers and 83% of Caucasian poor metabolizers. Studies have demonstrated an excellent correlation between a homozygous presence of mutant alleles and poor metabolizer status. An example of a procedure for genotyping CYP2C19 involves a series of polymerase chain reaction—restriction fragment length polymorphism reactions designed to detect nucleotide point mutations, deletions and insertions compared with the functional CYP2C19*1 allele (Furuta et al. (1999) Clin Pharmacol Thera 65(5):552-561; Tanigawara et al. (1999) Clin Pharmacol Thera 66(5):528-5534). PCR amplification of exon 5 or exon 4 for CYP2C19*2 and CYP2C19*3 respectively are performed using the following primers:

| | |
|---|---|
| CYP2C19*3 EXON 4 PRIMERS | |
| 5′-AACATCAGGATTGTAAGCAC-3′ | (SEQ ID NO:12) |
| 5′-TCAGGGCTTGGTCAATATAG-3′ | (SEQ ID NO:13) |
| CYP2C19*2 EXON 5 PRIMERS | |
| 5′-AATTACAACCAGAGCTTGGC-3′ | (SEQ ID NO:10) |
| 5′-TATCACTTTCCATAAAAGCAAG-3′ | (SEQ ID NO:11) |

The presence of the G681A mutation in CYP2C19*2 is then detected by digestion with the SmaI restriction enzyme. The wild type allele will produce a 120 and a 49 bp fragment, while the CYP2C19*2 allele will remain uncleaved. The CYP2C19*3 allele is detected by incubating the exon 4 PCR product with BamHI. The wild type allele will produce a 233 bp and a 96 bp fragment while the CYP2C19*3 allele will remain uncleaved.

Extensive metabolizing phenotype is assigned to those individuals with at least one allele encoding a functional enzyme. The poor metabolizing phenotype is assigned to individuals lacking two or more functional CYP2C19 alleles.

CYP2C9

The CYP2C9 family of metabolic enzymes accounts for approximately 8% of the metabolic enzymes in the liver. CYP 2C9 has been postulated as participating in approximately 15% of drug metabolism.

Polymorphism

Individuals are genetically polymorphic with respect to CYP 2C9 metabolism. Two metabolic phenotypes can be distinguished: extensive and poor metabolizers. Three genetic polymorphisms have been definitively identified, one wild type (CYP2C9*1) and two mutant (CYP2C9*2 and CYP2C9*3). The CYP2C9*2 allele was found to result in 5-10 fold increase in expression of mRNA and have 3-fold higher enzyme activity for metabolism of phenytoin and tolbutamide. Conversely, this genotype appears to have a lower level of activity for the metabolism of S-warfarin. The CYP2C9*3 allele appears to demonstrate decreased metabolic activity against all three of these substrates.

CYP2C9 metabolizes a variety of compounds including S-warfarin, phenytoin, tolbutamide, tienilic acid, and a number of nonsteroidal antiinflammatory drugs such as diclofenac, piroxicam, tenoxicam, ibuprofen, and acetylsalicylic acid.

Induction and Inhibition

CYP2C9 is inhibited by fluconazole, metronidazole, miconazole, ketoconazole, itaconazole, ritonavir, clopidrogel, amiodarone, fluvoxamine, sulfamthoxoazole, fluvastatin and fluoxetine. It is induced by rifampin and rifabutin.

Inter Ethnic Differences

The CYP2C9 genotypes demonstrate marked inter ethnic variability. The CYP2C9*2 is absent from Chinese and Taiwanese populations and present in only 1% of African American populations, but accounts for 19.2% of the British population and 8% of Caucasians. CYP2C9*3 is rarer and is present in 6% of Caucasian, 2% of Chinese, 2.6% of Taiwanese and 0.5% of African-American populations.

It is reasonable that, in drug metabolism studies, each ethnic group can be studied separately for evidence of polymorphism and its antimode should not be extrapolated from one ethnic population to another.

S-warfarin

As an example, the benefit of CYP2C9 metabolic phenotyping in drug dosing is evident in the case of S-warfarin. S-warfarin is an anticoagulant drug. Studies have demonstrated that the presence of either CYP2C*2 or CYP2C9*3 haplotypes results in a decrease in the dose necessary to acquire target anticoagulation intensity. In addition, these individuals also suffered from an increased incidence of bleeding complications. Therefore, the CYP2C9 gene variants modulate the anticoagulant effect of the dose of warfarin prescribed. For these reasons, the utility of a reliable test for CYP2C9 is evident. In particular, an accurate and convenient clinical assay would allow physicians to quickly identify safe and effective treatment regimes for individuals on an individual basis.

Direct Phenotypic Determinants of CYP2C9

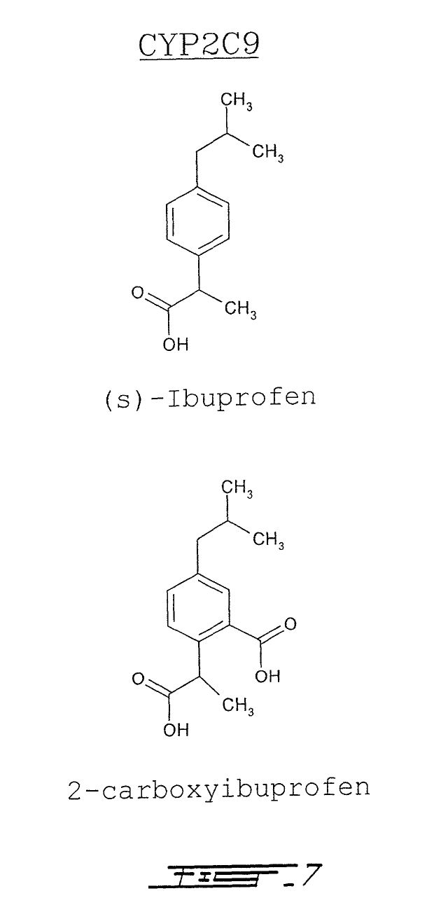

In accordance with an embodiment of the present invention, the ratio of (S)-ibuprofen and its carboxylated metabolite, (S)-2-carboxyibuprofen in a urine sample may be used to provide a determination of an individual's CYP2C9 phenotype. These metabolites are used as quantitative markers in the determination of a CYP2C9 phenotype on the basis of the use of the preferred probe substrate (S)-ibuprofen. The structures of (S)-ibuprofen and its metabolite (S)-2-carboxyibuprofen are illustrated in FIG. 7. However, it is fully contemplated that the present invention is not limited in any respect thereto. In fact, due to the nature of the substrate specific alterations caused by the individual CYP2C9 mutations, multiple probe substrates may be necessary for a completely informative phenotypic determination of CYP2C9.

The molar ratio of (S)-ibuprofen and its (S)-2-carboxyibuprofen metabolite, used to determine the CYP2C9 phenotype of the individual, is as follows:

$$\frac{( S ) \text{-} ibuprofen}{( S ) \text{-} 2 \text{-} carboxyibuprofen}$$

Indirect Phenotypic Determinants of CYP2C9 (Genotyping)

As mentioned previously the CYP2C9 has two predominant variant alleles, CYP2C9*2 and CYP2C9*3. An example of a procedure for genotyping CYP2C9 involves a series of polymerase chain reaction—restriction fragment length polymorphism reactions designed to detect nucleotide point mutations, deletions and insertions compared with the functional CYP2C9*1 allele (Taube et al. (2000) Blood 96(5):1816-1819). PCR amplification of exon 3 for CYP2C9*2 is performed using the following primers:

| | |
|---|---|
| CYP2C9*2 EXON 3 PRIMERS | |
| 5′-CAATGGAAAGAAATGGAAGGAGGT-3′ | (SEQ ID NO:14) |
| 5′-AGAAAGTAATACTCAGACCAATCG-3′ | (SEQ ID NO:15) |

A forced mismatch was included in the penultimate base of the forward primer to create a restriction site for the AvaII digestion. The PCR product from this amplification is 251 bp in length. After AvaII digestion the CYP2C9*1 (wt) allele produces 170 and 60 bp fragments. The CYP2C*2 allele produces a 229 bp fragment.

The CYP2C9*3 allele does not naturally destroy or produce a restriction site. Therefore, a restriction site was introduced into the forward primer such that the adenosine at position 1061 (A1061) in combination with the mismatch creates a restriction site for the NsiI restriction enzyme. Therefore the PCR amplified fragment of the CYP2C9*1 (wt) allele would have a restriction site at A1061. Conversely, the mutation of A1061C in CYP2C9*3 removes this restriction site. The forward primer also includes a natural AvaII restriction sequence. The reverse primer also has a forced mismatch at 1186 to provide a restriction site for the NsiI restriction enzyme (PCR amplified fragments from both the CYP2C9*1 and CYP2C9*3 alleles will have this restriction site). The PCR product for this set of primers prior to restriction enzyme digest is 160 bp in length. Following restriction digest with NsiI and AvaII, the CYP2C9*1 allele produces a 130 bp fragment and the CYP2C9*3 allele produces a 140 bp fragment.

| | |
|---|---|
| CYP2C9*3 PRIMERS | |
| 5′-TGCACGAGGTCCAQAGATGC-3′ | (SEQ ID NO:16) |
| 5′-AGCTTCAGGGTTTACGTATCATAGTAA-3′ | (SEQ ID NO:17) |

Due to the substrate specific alterations in enzyme activity resulting from the two allelic variants, the phenotypic determination will be correlated on an individual substrate basis.

CYP2D6

CYP2D6 constitutes 1-3% of the total CYP 450 enzymes in the human liver. CYP2D6 has been postulated as participating in ˜20% of drug metabolism.

Polymorphism

CYP2D6 was the first P450 enzyme to demonstrate polymorphic expression in humans. Three metabolic phenotypes can be distinguished: poor, (PM), extensive (EM) and ultraextensive (UEM) phenotypes. The CYP2D6 gene is extensively polymorphic. For example, a 1997 study documented 48 mutations and 53 alleles of the CYP2D6 gene in a screen of 672 unrelated individuals. Examples of alleles with normal (extensive), wild-type function are CYP2D6*1, CYP2D6*2A, and CYP2D6*2B; alleles resulting in an absence of function are CYP2D6*3, CYP2D6*4A, CYP2D6*4B, CYP2D6*5, CYP2D6*6A, CYP2D6*6B, CYP2D6*7, CYP2D6*8, CYP2D6*11 and CYP2D6*12; and alleles resulting in a reduced function are CYP2D6*9, CYP2D6*10A, and CYP2D6*10B. The ultraextensive phenotype appears to arise from the presence of multiple copies of the CYP2D6 gene (for example, one individual was identified with 13 copies of the gene).

CYP2D6 metabolizes a large variety of drugs and dietary constituents including, but not limited to the following:

Antiviral Agents:

Efavirenz, nevirapine,ritonavir, saquinovir, nelfinavir mesylate, and indinavir

Psychotropic Drugs:

amiflamine, amitryptyline, clomipramine, clozapine, desipramine, haloperidol, imipramine, maprotiline, methoxyphenamine, minaprine, nortriptyline, paroxetine, perphenazine, remoxipride, thioridazine, tomoxetine, trifluperidol, zuclopenthixol, risperidone, and fluoxetine.

Cardiovascular Agents:

aprindine, bufuralol, debrisoquine, encainide, flecainide, guanoxan, indoramin, metoprolol, mexiletin, n-propylamaline, propafenone, propranolol, sparteine, timolol, and verapamil.

Miscellaneous Agents:

chlorpropamide, codeine, dextromethorphan, methamphetamine, perhexilene, and phenformin.

In addition, CYP2D6 is involved in the metabolism of many carcinogens, however, as yet it is not reported as the major metabolizer for any. In one study it has been shown that individuals who are fast CYP2D6 metabolizers and slow N-acetylators are at a greater risk for hepatocellular cancer (OR=2.6; 95% CI=1.6-4).

Induction and Inhibition

CYP2D6 is inhibited in vitro by quinidine and by viral protease inhibitors as well as by appetite suppressant drugs such as D- and L-fenfluramine.

Inter Ethnic Differences

The activity of CYP2D6 varies broadly in a given population. Poor (PM), extensive (EM) and ultraextensive (UEM) phenotypes of CYP2D6 have been distinguished. The CYP2D6 gene is inherited as an autosomal recessive trait and separates 90 and 10% of the white European and North American population into extensive (EM) and poor (PM) metabolizer phenotypes, respectively. In another study the percentage of PM in different ethnic populations was observed, and white North Americans and Europeans were found to have 5-10% PM's, African-American, 1.8%, Native Thais, 1.2%, Chinese 1%, and Native Malay populations, 2.1%, while the PM phenotype appears to be completely absent in the Japanese population. It is reasonable that, in drug metabolism studies, each ethnic group can be studied separately for evidence of polymorphism and its antimode should not be extrapolated from one ethnic population to another.

Dextromethorphan/Antidepressants

An example of the need for phenotyping in drug dosing is the case of dextromethorphan. Dextromethorphan is a nonoploid antitussive with psychotropic effects. However, dextromethorphan doses range from 0 to 6 mg/kg based on individual tolerance. Dextromethorphan is activated via the CYP2D6 metabolizing system. Dextromethorphan produced qualitatively and quantitatively different objective and individualive effects in poor vs. extensive metabolizers (mean performance +/−SE, 95+/−0.5% for EMs vs. 86+/−6% for PMs; p<0.05).Another important class of drugs for CYP2D6 phenotyping is the tricyclic antidepressants. Both the PM and UEM phenotypes of CYP2D6 are at risk of adverse reactions. PM individuals given standard doses of these drugs will develop toxic plasma concentrations, potentially leading to unpleasant side effects including dry mouth, hypotension, sedation, tremor, or in some cases life-threatening cardiotoxicity. Conversely, administration of these drugs to UEM individuals may result in therapeutic failure because plasma concentrations of active drugs at standard doses are far too low. For, these reasons, the utility of a reliable phenotyping test for CYP2D6 is evident

Phenotypic Determinants of CYP2D6

Different probe substrates can be used to determine the CYP2D6 phenotype (dextromethorphan, debrisoquine, bufuralol, antipyrine, theophylline and hexobarbital). In accordance with the present invention, suitable probe substrates include without limitation, dextromethorphan, debrisoquine, and bufuralol.

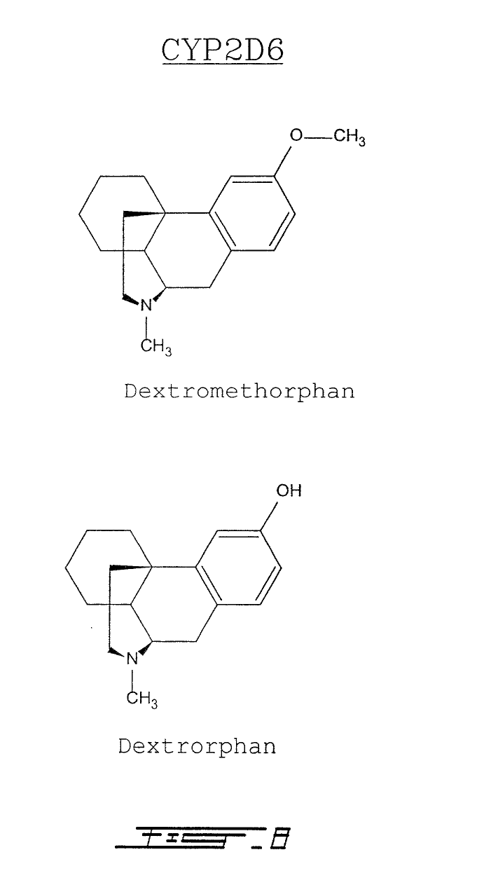

Of these dextromethorphan is the preferred probe substrate. The structure of dextromethorphan and its demethylated metabolite dextrorphan are illustrated in FIG. 8.

In accordance with the present invention, the molar ratio of dextromethorphan and its metabolite is used to determine the CYP2D6 phenotype of the individual as follows:

$$\frac{dextromethorphan}{dextrorphan}$$

An antimode of 0.30 is used to differentiate between extensive and poor metabolizers whereby an antimode of less than 0.30 indicates an extensive metabolizer and greater than 0.30 indicates a poor metabolizer.

Indirect Phenotypic Determinants of CYP2D6 (Genotyping)

As mentioned previously the CYP2D6 gene is extensively polymorphic with one study identifying 48 mutations and 53 alleles. An example of a procedure for genotyping CYP2D6 involves the amplification of the entire CYP2D6 coding region (5.1 kb product) by XL-PCR using specific primers. This product is then used for a series of polymerase chain reaction—restriction fragment length polymorphism reactions designed to detect nucleotide point mutations, deletions and insertions compared with the functional CYP2D6*1 allele (Garcia-Barceló et al. (2000) Clinical Chemistry 46(1):18-23). For example, to detect the C188T transition mutation the following primers can be used to first amplify the CYP2D6 gene and then the specific region of the mutation:

| | |
|---|---|
| FULL CYP2DG GENE | |
| 5′-CCAGAAGGCTTTGCAGGCTTCA-3′ | (SEQ ID NO:18) |
| 5′-ACTGAGCCCTGGGAGGTAGGTA-3′ | (SEQ ID NO:19) |
| C188T MUTATION | |
| 5′-CCATTTGGTAGTGAGGCAGGTAT-3′ | (SEQ ID NO:20) |
| 5′-CACCATCCATGTTTGCTTCTGGT-3′ | (SEQ ID NO:21) |

The presence of the C188T mutation is then detected by digestion with the HphI restriction enzyme.

In general, the most frequent mutations are examined and these correspond to the most frequent alleles and genotypes.

Extensive metabolizing phenotype is assigned to those individuals with at least one allele encoding a functional enzyme. The poor metabolizing phenotype is assigned to individuals lacking two or more functional CYP2D6 alleles.

CYP2E1

CYP2E1 constitutes approximately 5% of the total CYP 450 enzymes in the human liver.

Polymorphism

The CYP2E1 gene has been demonstrated to be polymorphic in the human population. Studies have demonstrated the presence of 10 CYP2E1 alleles (one wt CYP2E1*1, and 9 mutant, CYP2E1*2, CYP2E1*3, CYP2E1*4, CYP2E1*5A, CYP2E1*5B, CYP2E1*6, CYP2E1*7A, CYP2E1*7B, and CYP2E1*7C). The exact relationship of these polymorphisms to CYP2E1 enzyme activity has not been clarified, however, some studies suggest that the mutant alleles CYP2E1*5A and CYP2E1*5B, result in increased transcription and increased enzyme activity.

CYP2E1 metabolizes several drugs and dietary constituents including isoflurane, halothane, methoxyflurane, enflurane, propofol, thiamylal, sevoflurane, ethanol, acetone, acetaminophen, nitrosamines, nitrosodimethylamine, and p-nitrophenol.

In addition, CYP2E1 activates environmental pro-carcinogens especially nitrosodimethylamine, nitrosopyrrolidone, benzene, carbon tetrachloride, and 3-hydroxypyridine (tobacco smoke product). In one study it has been shown that individuals who have high CYP2E1 (CYP2E1*5A or CYP2E1*5B) activity are at a greater risk for gastric cancer (OR=23.6-25.7).

Induction and Inhibition

CYP2E1 is induced by a number of drugs and environmental factors such as cigarette smoke as well as by starvation, chronic alcohol consumption and in uncontrolled diabetes. CYP2E1 is inhibited by chlormethiazole, trans-1,2-dichloroethylene, disulferan (cimetidine) and by the isoflavonoids genistein and equol.

Induction or inhibition by environmental factors can severely alter an individual's capacity to metabolize certain drugs. Therefore, the present invention may find further application in the individualization of therapy whereby environmental factors are determined to effect an individual's metabolism specific to an enzyme and/or metabolic pathway of interest with respect to a given drug, such as CYP2E1, for example. Furthermore, as environmental factors vary on an individual basis and over time, the present invention may be employed to detect changes in an individual's metabolism specific to an enzyme and/or metabolic pathway of interest due to environmental factors at any given time, and provide valuable phenotype-specific information in the determination of a safe and efficacious individualized treatment regime. By employing the present invention on a routine basis, an individual's treatment regime may be modified to account for environmental influences and maximize the effectiveness of treatment.

Inter Ethnic Differences

The proportion of CYP2E1 phenotypes varied between ethnic groups and countries: the frequency of the rare c2 (CYP2E1*5A or CYP2E1*5B) allele is about 4% in Caucasians and 20% in the Japanese and a study of a separate polymorphism described a rare C allele (CYP2E1*5A or CYP2E1*6) that has a frequency of about 10% in Caucasian and 25% in Japanese populations. In one study it was shown that Japanese males had much lower levels of CYP2E1 activity as compared to Caucasian males. Therefore, it is reasonable that, in drug metabolism studies, each ethnic group can be studied separately for evidence of polymorphism and its antimode should not be extrapolated from one ethnic population to another.

Acetaminophen

An example of the need for phenotyping in drug dosing is the case of acetaminophen. Acetaminophen is a widely used painkiller. However, acetaminophen causes hepatotoxicity at low frequency. The hepatotoxicity is due to its transformation via CYP2E1, to a reactive metabolite (N-acetyl-p-benzoquinoneimine) which is capable of binding to nucleophiles. For these reasons, the utility of a reliable phenotyping test for CYP2E1 is evident.

Direct Phenotypic Determinants of CYP2E1

In accordance with the present invention a suitable probe substrate is, without limitation, chlorzoxazone.

In accordance with the present invention, the molar ratio of chlorzoxazone and its metabolite is used to determine the CYP2E1 phenotype of the individual as follows:

$$\frac{6 \text{-} hydroxychlorzoxazone}{chlorzoxazone}$$

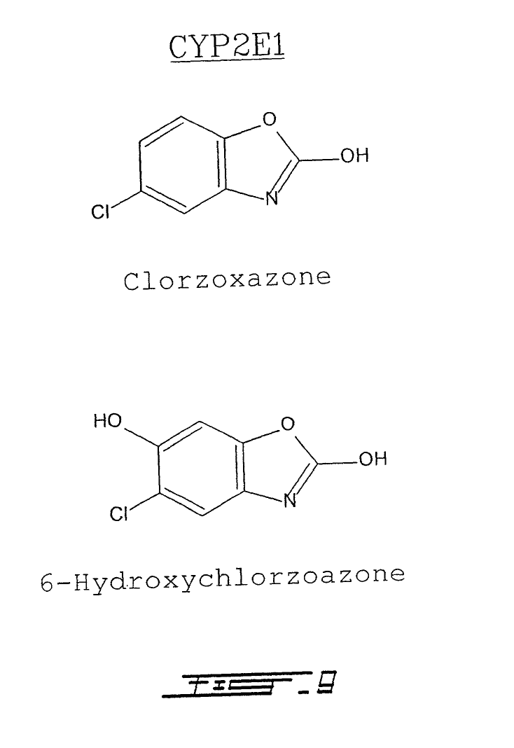

The structures of chlorzoxazone and its metabolite 6-hydroxychlorzoxazone are illustrated in FIG. 9.

Indirect Phenotypic Determinants of CYP2E1 (Genotyping)

As mentioned previously the CYP2E1 gene has multiple polymorphisms. An example of a procedure for genotyping CYP2E1 for the most common mutations, those termed the Pst/RsaI and DraI mutations (allows genotyping of CYP2E1*5A, CYP2E1*5B and CYP2E1*6), involves the amplification of a fragment containing either the PstI and RsaI restriction sites or the DraI restriction site using specific primers (Nedelcheva et al. (1996) Methods in Enzymology 272:218-225). The amplified product is then incubated with the appropriate restriction enzyme (PstI or RsaI/DraI) and the digestion products separated electrophoretically. From an allele with wt sequence at the PstI or RsaI site, the 510 bp fragment produced by PCR is cleaved to a 360 bp and a 150 bp fragment. From the mutant allele the 510 bp fragment remains uncleaved. From an allele with the wt sequence at the DraI mutation site, the 370 bp PCR amplified fragment is cleaved to a 240 bp and 130 bp pair of fragments, while the mutant allele is uncleaved.

| | |
|---|---|
| PSTT/RSAI PRIMERS | |
| 5′-CCCGTGAGCCAGTCGAGT-3′ | (SEQ ID NO:22) |
| 5′-ATACAGACCCTCTTCCAC-3′ | (SEQ ID NO:23) |
| DRAI PRIMERS | |
| 5′-AGTCGACATGTGATGGATCCA-3′ | (SEQ ID NO:24) |
| 5′-GACAGGGTTTCATCATGTTGG-3′ | (SEQ ID NO:25) |

The CYP2E1*5A mutant allele contains both the RsaI and the DraI mutations, while the CYP2E1*5B mutant allele contains the RsaI mutation alone. The RsaI mutation has been associated with an increased expression and increased enzyme activity. Therefore, an individual with two copies of either CYP2E1*5 allele could be considered assigned an extensive metabolizing phenotype. Conversely, the CYP2E1*2 mutation has been associated with decreased protein expression and decreased enzyme activity. Therefore, a person homozygous for the CYP2E1*2 allele could be assigned a poor metabolizing phenotype.

Characterization of Multiple Phenotypic Determinants

On the basis of the above enzyme-specific metabolic pathways, several approaches to identifying phenotypic determinants thereof have been developed in accordance with the present invention. The characterization of multiple phenotypes offers multiple applications. The determination of an individual's metabolic phenotype for a multitude of cytochrome P450 and N-acetyltransferase metabolic enzymes allows the use of this single profile for multiple applications. If a drug is metabolized by more than one enzyme, the phenotypic status of each of the enzymes may be important for first, determining if the individual can safely ingest a given drug and second, determining the optimal dose for this individual if they are able to take the drug.

For example, in the case of the antineoplastic agent amonafide, it is suggested that CYP1A2 may, in addition to NAT2, play a minor but nonetheless significant role in the metabolism of this drug. Accordingly, it is contemplated that the ability to characterize multiple phenotypic determinants may also play an important role in the individualization of therapy with amonafide on the basis of phenotyping.

In addition, the knowledge of multiple phenotypes will facilitate the comparison of multiple drugs within the same class or genus, where different metabolic enzymes are involved in the metabolism of these drugs. For example, consider an individual requiring a certain class of drug, of which there are three that are primarily prescribed. If one is metabolized by CYP1A2, one by CYP2D6 and the remaining drug by CYP3A4, and all individuals that are poor metabolizers of these drugs are at risk for toxicity. Then the drug chosen for treating that individual may be determined on the basis of a phenotypic profile of that individual. If for example the individual is a poor metabolizer for CYP2D6 and CYP3A4, then the first drug metabolized by CYP1A2 may be the first drug to consider for treating the individual.

Another advantage to the determination of an individual's metabolic profile for multiple phenotypic determinants is the effect of a drug on the metabolic status of enzymes not primarily involved in its metabolism. For example, a drug may be metabolized by CYP2C9 and inhibit the activity of CYP3A4. If an individual has very low levels of CYP3A4 to begin with then this inhibition may have little effect on that individuals CYP3A4 phenotype. However, if the individual is an extensive CYP3A4 metabolizer this drug may profoundly alter the CYP3A4 metabolic status. This can cause enormous problems in the case of polypharmacy, where an individual may be taking multiple drugs, and the addition of one drug may affect the safety and efficacy of the pre-existing drug treatment(s).

The metabolic phenotype can be determined directly (by measuring enzyme activity) or indirectly (by examining enzymes genetic sequence). In general, for example, for direct phenotyping, a probe substrate or substrates, such as those exemplified in Table 1 are administered to an individual to be phenotyped. A biological sample, such as a urine sample is subsequently collected from the individual approximately 4 hours after administering the probe substrate(s). The urine sample is analyzed according to a ligand binding assay, such as enzyme-linked immunosorbent assay (ELISA) technology as described hereinbelow, for metabolites corresponding to the probe substrate(s) and the molar ratios of the metabolites calculated to reveal the individual phenotypes.

In general, for example, for indirect phenotyping, a blood sample of an individual is obtained, and the genetic sequence of the enzyme(s) is examined for the presence or absence of specific mutations. A specific probe for a known allelic variation may be used to screen for a specific genotype known to effect an individual's specific enzymatic capacity. The combination of mutations on the two alleles is matched to known genotypes. The phenotype is then inferred for those genotypes whose presence has been correlated to a known phenotype.

Ligand-binding Assays

The specificity of the molecular recognition of antigens by antibodies to form a stable complex is the basis of both the analytical immunoassay in solution and the immunosensor on solid-state interfaces. The underlying fundamental concept of these analytical methods as ligand-binding assays is based on the observation of the products of the ligand-binding reaction between the target analyte and a highly specific binding reagent.

The development of immunoassay technology is a success story especially for the clinical laboratory and still continues to be a vibrant area of research. Further development and automation will expand the possibilities of immunoassay analysis in the clinical sciences. Besides this, new areas for trace analyses using immunoassay were defined in the last decade: the environmental analysis of trace substances and quality control in the food industry. Since these applications also need a continuous monitoring mode, the idea of an immunosensor as a continuously working heterogeneous immunoassay system, covering these features, was conceived. The immunosensor is now considered as a major development in the immunochemical field. Despite an overwhelming number of papers is this field, there are only a few commercial applications of immunosensors in clinical diagnostics. The reasons are, in part, unresolved fundamental questions relating to immobilization, orientation, and specific properties of the antibodies or antibody-related reagents on the transducer surface. In addition, a key issue is which clinical applications may benefit most from immunosensor devices in the routine medical laboratory. only if there is consensus on the clinical utility of this new technique can the gap between the high expectations of the developer and reality be closed. Designers of immunosensor devices must be aware of the general and special needs of laboratory medicine from new analytical techniques.

A new analyzer should be simple and “rugged” for the measurement of analytes. Measurements have to be performed precisely and accurately, even under emergency conditions. The analyzer must be fully automated and capable of performing rapid measurements with turnaround times of <1 h. Additionally, the determination of an analyte should preferably be without sample pretreatment in matrices, such as serum, plasma, urine or cerebrospinal fluid. All parameters determined with a new analyzer must meet the following criteria, which are defined in various guidelines: low imprecision, small lot-to-lot variations, high analytical sensitivity, optimum analytical specificity and accuracy with long calibration stability and low interferences by drugs or normal and pathological sample components.

In the clinical laboratory, a future substitution of immunoassays by immunosensors simply depends on the superiority and versatility of the new methodology. The applicability for point-of-care testing or when they are temporarily implanted into the individual additionally depends on the reliable and accurate analysis of the desired analyte, without drift problems or matrix interferences. Due to the tremendously growing variety of developments, this review is not intended to be comprehensive. Hence, the main focus will be the description and assessment of reported clinical applications of immunosensors. For a more thorough understanding, we refer to several excellent reviews in the last 5 years on technical aspects and the application of immunosensors in various fields. Other related reviews deal with antibody engineering developments and latest immunoassay technologies.

Antibodies as Bioaffinity Interface for Both Immunoassays and Immunosensors

It should first be clarified that the specificity for the measurement of analytes in all immunosensor systems, as in the case of immunoassays, is dependent on the application of affinity complexation agents (binding molecules). This pivotal feature is shared by both technologies. New developments in protein engineering for immunoglobulins (including antibody fragments, and chimeric antibodies) or in substituting antibodies by alternative binding components (aptamers are one example) or structures (molecular imprinting is one example) will, therefore, be applicable to either technology, if available. In particular, the possibilities in antibody engineering will enable changes in the affinity and fine specificity of antibodies, as well as the expression of fragments as fusion proteins coupled to reporter molecules.

Immobilization Procedures for Antibodies

Antibodies have to be properly immobilized on the immunosensor surface, which is mostly part of a flow-through cell. The optimum density and adjusted (but not random) orientation of the antibodies are of paramount importance. Due to the different types of sensing surfaces, this manipulation can have benefits e.g., improvement of the reaction kinetic parameters, but also unfavorable effects (e.g., increased nonspecific binding, partly destroyed paratope). There are four different types of oriented coupling of antibodies: binding to Fc receptors such as protein A or G or recombinant ArG fusion protein on the surface; binding of other binding partners to structures, covalently linked to the Fc part of the antibody, e.g., the biotin residue on the Fc binds to surface-coated streptavidin; coupling to the solid support via an oxidized carbohydrate moiety on the C2 Fc domain; and the binding of Fab or scFv fragments to the surface of the device via a sulfhydryl group in its C-terminal region.

Numerous chemical reactions can be applied to the immobilization onto solid surfaces. Defined linkages between the antibody or its carbohydrate moieties and the solid phase material (silica, silanized silica, Ta- or Ti-oxides, plastics, sepharose, and metal films) are being built by glutaraldehyde, carbodiimide, uccinimide ester, maleinimide, periodate or galactose oxidase. Moreover, photo-immobilization of antibodies using albumin derivatized with aryldiaziridines as photolinker, is applicable. Physiosorption is not recommended due to the local instability of the layer caused by the mechanical stress in the flow-through cell. An exciting new method for antibody immobilization on a quartz surface of a piezoelectric sensor is based on the deposition of an ethylenediamine plasma polymerization film on the quartz crystal. This film is extremely thin and homogeneous, incorporating amino functions which may be further derivatized and linked to immunoglobulins, resulting in an orientation-controlled and highly reusable sensing surface. Another recent development is the planar-supported phospho-lipid bilayer (SLB), which can be formed on solid supports by vesicle fusion and Langmuir-Blodgett methods. SLBs maintain two-dimensional fluidity and accommodate multivalent binding between surface-bound ligands and receptor molecules in solution.

For noble metal surfaces, such as gold, in particular, in optical immunosensors, self-assembling monolayer (SAM) techniques seem to be first choice. In general, a SAM is built of long-chained (C12 and higher) n-alkylthiols with derivatized organic functional groups, which are easily linked to the gold film via the thiol groups by a mechanism still not fully understood. The functional groups of the SAM cross-link with the Fc portion of the antibody (one way is via the biotin streptavidin system), whereas the self-organization of the matrix prevents the surface being individualed to nonspecific binding effects. In addition, the covalent coupling of IgG to a short-chain (thioctic or mercaptopropionic acid are two examples) SAM-modified metal surface has been shown to be an effective affinity-based layer for optical immunosensors.

Regeneration of Antibody-coated Sensor Surfaces

Conventional homogeneous and heterogeneous immunoassays, respectively, work discontinuously. It is highly desirable, however, that immunosensor devices, applied in clinical diagnostics, are capable of quasi-continuous recording. The repeated use of disposable sensing elements may mimic a pseudocontinuous action, but this is not considered here. In true immunosensors, the analyte/antigen interaction on the sensor-coated surface is reversible. With the given short incubation times in the flow-through device, the reaction between antigen and antibody is far off the equilibrium state. Fast reversibility and high sensitivity are mutually exclusive of each other. Consistently, an adequate analytical sensitivity is only warranted if antibodies with increased affinity >10¹⁰ M⁻¹ or at least with highly improved on-rate are applied.

The regeneration of the binding sites of the antibodies bound to the immunosensor surface needs stringent procedures. Antibody regeneration using acidic or alkaline solutions, guanidinium chloride, or ionic strength shock is potentially harmful to the binding ability and may lead to a diminished lifetime of the immobilized antibodies and insidious drift problems.

Besides this, it must be considered that with the short reaction times between the antibodies and soluble analytes in the flow-through system, the cross-reactivities of the antibody applied can be increased. A highly specific recognition of the antigen is a kinetic-controlled process due to the complexity of the conformational changes in the Fab portion of the antibody upon binding of the antigen.

There are different approaches to solve the “antibody regeneration” problem: one approach is to displace the antigenic analyte by a highly concentrated solution of a related antigen with weak affinity to the surface-bound antibody. However, this depends on the availability of a suitable antigenic surrogate. This is not always feasible and is only applicable to small analytes. A second approach is to use the techniques of antibody engineering to improve the chemical stability of antibodies as whole molecules or as Fab fragments. The phage display technique is such a powerful tool. This can be helpful in the selection of antibody fragments with improved stability. Libraries of mutants of single-chain Fv fragments (scFv), comprising the variable regions of the L and H chains, joined by a peptide linker are generated by a combination of site-directed and random mutagenesis. The selection can be carried out under different physical or chemical pressures to produce thermodynamically more stable scFv mutants. An interesting third approach is a pseudo-regenerating procedure for immunosensors. An amperometric sensor is coated with a conducting immunocomposite, formed by a mixture of specific antibody with methacrylate monomer and graphite. After polymerization, the device is ready for use. Repeated measurements became possible if the polymer is polished thoroughly with abrasive paper. These notes do not apply to immunosensors with a competitive configuration, in which antigenic compounds and not antibodies are surface-immobilized.

Alternative Analyte-binding Compounds for Immunosensor Applications

Aptamers

Aptamers are single-stranded DNA or RNA oligonucleotide sequences with the capacity to recognize various target molecules with high affinity and specificity. These ligand-binding oligonucleotides mimic properties of antibodies in a variety of diagnostic formats. They are folded into unique overall shapes to form intricate binding furrows for the target structure. Aptamers are identified by an in vitro selection process known as systematic evolution of ligands by exponential enrichment (SELEX). Aptamers may have advantages over antibodies in the ease of depositing them on sensing surfaces. Moreover, due to the highly reproducible synthetic approach in any quantities, albeit the affinity constants are consistently lower than those of antibodies and the stability of these compounds is still questionable, they may be particularly useful for diagnostic applications in complex biological matrices. The aptamer-based schemes are still in their infancy and it is expected that modified nuclease-resistant RNA and DNA aptamers will soon be available for a variety of therapeutic and diagnostic formats. The potential of aptamers for use in biosensors has been outlined in the design of a fiber-optic biosensor using an anti-thrombin DNA aptamer, immobilized on the surface of silica microspheres and distributed into microwells on the distal tip of the imaging fiber. With this device, the determination of thrombin at low concentration was possible. Exciting new possibilities are evolving by the introduction of signaling aptamers with ligand-dependent changes in signaling characteristics and catalytically active so-called “apta-zymes” which would allow the direct transduction of molecular recognition to catalysis.

Anticalins

Lipocalins constitute a family of proteins for storage or transport of hydrophobic and/or chemically sensitive organic compounds. The retinol-binding protein is an example in human physiology. It has been demonstrated that the bilin-binding protein, a member of the lipocalin family and originating from the butterfly Pieris brassicae, can be structurally reshaped in order to specifically complex potential antigens, such as digoxigenin, which was given as an example. These binding proteins share a conserved β-barrel, which is made of eight antiparallel β-strands, winding around a central core. At the wider end of the conical structure, these strands are connected in a pairwise manner by four loops that form the ligand binding site. The lipocalin scaffold can be employed for the construction of so-called “anticalins”, which provide a promising alternative to recombinant antibody fragments. This is made by individualizing various amino acid residues, distributed across the four loops, to targeted random mutagenesis. It remains to be shown that this class of proteins is applicable in diagnostic assays and in immunosensors. Critical points that still need to be defined include the synthesis and stability of the anticalins, the magnitude of the affinity constants, and the versatility for being crafted against the large variety of ligands.

Molecular Imprinting Techniques

This is a technique that is based on the preparation of polymeric sorbents which are selectivity predetermined for a particular substance, or group of structural analogs. Functional and cross-linking monomers of plastic materials, such as methacrylics and styrenes, are allowed to interact with a templating ligand to create low-energy interactions. Subsequently, polymerization is induced. During this process, the molecule of interest is entrapped within the polymer either by a noncovalent, self-assembling approach, or by a reversible, covalent approach. After stopping the polymerization, the template molecule is washed out. The resultant imprint of the template is maintained in the rigid polymer and possesses a steric (size, shape) and chemical (special arrangement of complementary functionality) memory for the template. The molecularly imprinted polymer (MIP) can bind the template (=analyte) with a specificity similar to that of the antigen-antibody interaction.

Besides the main applications in solid-phase extraction and chromatography, molecularly imprinted polymers have already been employed as nonbiological alternatives to antibodies in competitive binding assays. A series of applications for analytes, such as cyclosporin A, atrazine, cortisol, 17b-estradiol, theophylline, diazepam, morphine, and S-propranolol, suggests that molecular imprinting is a promising technique for immunoassays and immunosensors.

Immunoassay and Immunosensor Technologies

Immunosaaays

Immunoassays use antibodies or antibody-related reagents for the determination of sample analytes. This analytical tool has experienced an evolutionary history since 1959, when Berson and Yalow first described the radioimmunoassay (RIA) principle. In the RIA, a fixed and limited amount of antibody is reacted with a fixed and limited amount of radiolabeled antigen tracer and a variable concentration of the analyte. The selectivity of the ligand-binding of antibodies allows these biomolecules to be employed in analytical methods that are highly specific even in complex biological matrices, such as blood, plasma, or urine. By combining the selectivity of antibody-analyte interactions with the vast array of antibodies preformed in immunization processes of host animals and the availability of numerous readily detectable labels radioisotopes, enzymatically or electrochemically induced adsorbance or fluorescence or chemi-luminescence, immunoassays can be designed for a wide variety of analytes while with extraordinarily low detection limits.

Biosensors and Immunosensors

A biosensor is an analytical device that integrates a biological element on a solid-state surface, enabling a reversible biospecific interaction with the analyte, and a signal transducer. The biological element is a layer of molecules qualified for biorecognition, such as enzymes, receptors, peptides, single-stranded DNA, or even living cells. If antibodies or antibody fragments are applied as a biological element the device is called an immunosensor. Compared to conventional analytical instruments, biosensors are characterized by an integrated structure of these two components. Many devices are connected with a flow-through cell, enabling a flow-injection analysis (FIA) mode of operation. Biosensors combine high analytical specificity with the processing power of modern electronics to achieve highly sensitive detection systems.

There are two different types of biosensors: biocatalytic and bioaffinity-based biosensors. The biocatalytic biosensor uses mainly enzymes as the biological compound, catalyzing a signaling biochemical reaction. The bioaffinity-based biosensor, designed to monitor the binding event itself, uses specific binding proteins, lectins, receptors, nucleic acids, membranes, whole cells, antibodies or antibody-related substances for biomolecular recognition. In the latter two cases, these biosensors are called immunosensors.

Biosensors are extensively used as diagnostic tools, predominately in point-of-care testing. Probably the most successful commercialization of biosensors today is the in vitro near individual measurement of capillary glucose using various hand-held systems with disposable reagent cartridges.

Immunosensor Principles

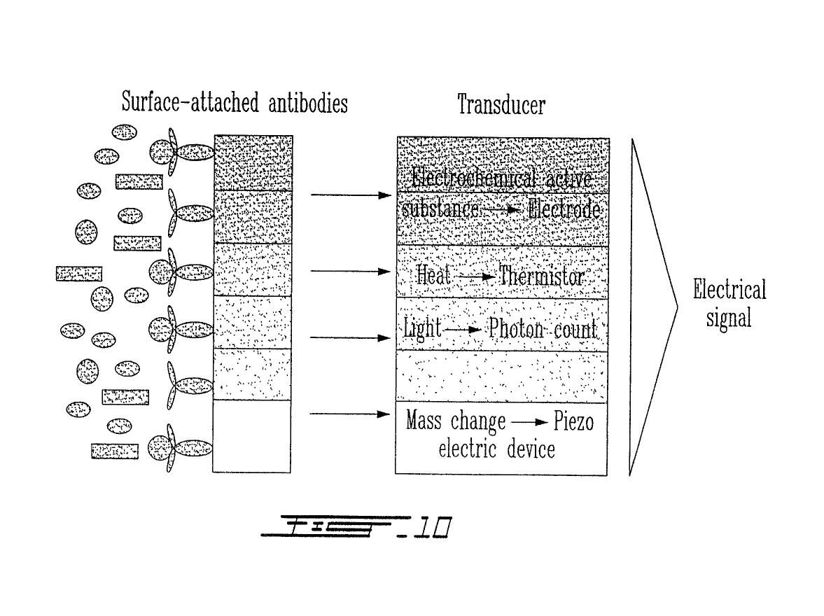

The general immunosensor design is depicted in FIG. 10. There are four types of immunosensor detection devices: electrochemical (potentiometric, amperometric or conductometric/capacitative), optical, microgravimetric, and thermometric. All types can either be run as direct nonlabeled or as indirect labeled immunosensors. The direct sensors are able to detect the physical changes during the immune complex formation, whereas the indirect sensors use signal-generating labels which allow more sensitive and versatile detection modes when incorporated into the complex.

There is a great variety of different labels which have been applied in indirect immunosensors. In principle they are the same labels as used in immunoassays. Among the most valuable labels are enzymes such as peroxidase, glucose oxidase, alkaline phosphatase (AP), catalase or luciferase, electroactive compounds such as ferrocene or In²⁺salts, and a series of fluorescent labels (including rhodamine, fluorescein, Cy5, ruthenium diimine complexes, and phosphorescent porphyrin dyes). In particular, laser-induced fluorometric resonance energy transfer between two fluorophores offers methodological advantages and can be extended to fiberoptic sensing.

Although indirect immunosensors are highly sensitive due to the analytical characteristics of the label applied, the concept of a direct sensor device is still fascinating and represents a true alternative development to immunoassay systems. Its potential simplicity holds multiple advantages, making immunosensors progressive and future directed.

The present invention will be illustrated using the following examples, which are not to be seen as limiting in any way. Those skilled in the art will recognize, or be able to ascertain using no more than routine experimentation, many equivalents to the specific embodiments of the invention described specifically herein Such equivalents are intended to be encompassed in the scope of the claims.

Electrochemical Sensors

POTENTIOMETRIC IMMUNOSENSORS. The Nernst equation provides the fundamental principle of all potentiometric transducers. According to this equation, potential changes are logarithmically proportional to the specific ion activity. Potentiometric transducer electrodes, capable of measuring surface potential alterations at near-zero current flow, are being constructed by applying the following methodologies.

TRANSMEMBRANE POTENTIAL. This transducer principle is based on the accumulation of a potential across a sensing membrane. Ion-selective electrodes (ISE) use ion-selective membranes which generate a charge separation between the sample and the sensor surface. Analogously, antigen or antibody immobilized on the membrane binds the corresponding compound from the solution at the solid-state surface and changes the transmembrane potential.

ELECTRODE POTENTIAL. This transducer is similar to the transmembrane potential sensor. An electrode by itself, however, is the surface for the immunocomplex building, changing the electrode potential in relation to the concentration of the analyte.

FIELD-EFFECT TRANSISTOR (FET). The FET is a semiconductor device used for monitoring of charges at the surface of an electrode, which have been built up on its metal gate between the so-called source and drain electrodes. The surface potential varies with the analyte concentration. The integration of an ISE with FET is realized in the ion-selective field-effect transistor (ISFET). This technique can also be applied to immunosensors.

An advantage of potentiometric sensors is the simplicity of operation, which can be used for automation, and the small size of the solid-state FET sensors. All potentiometric methods, however, are still suffering from major problems of sensitivity, being inferior to amperometric transducers and nonspecific effects of binding or signaling influences from other ions present in the sample. Especially, the signal-to-noise ratio causes analytical problems, which are difficult to circumvent. Thus, a trend away from these techniques has been observed in the last few years. However, the ISFET may be seen as a candidate for ultrasensitive clinical immunosensor applications, in particular, when the novel concept of differential ISFET-based measurement of the zeta potential is used. The streaming potential is a potential difference in flow direction, caused by the flow of excess ions resulting from a local distortion of the charge balance. The zeta potential, directly correlated to the streaming potential, reflects the potential changes in the diffuse outer layer at the solid-liquid interface. It efficiently reacts to protein accumulations onto sensor surfaces and, thus, is suitable for detecting immunocomplex reactions.

Amperometric Immonusensors.

Amperometric immunosensors are designed to measure a current flow generated by an electrochemical reaction at constant voltage. There are only few applications available for direct sensing, since most protein analytes are not intrinsically able to act as redox partners in an electrochemical reaction. Therefore, electrochemically active labels directly or as products of an enzymatic reaction are needed for the electrochemical reaction of the analyte at the sensing electrode. Oxygen and H₂O₂ electrodes are the most popular. An oxygen electrode consists of an electrolyte-bearing chamber with a sensing Pt cathode, polarized at 0.7 V, and an Ag/AgCl reference electrode. The chamber is gas-permeable, covered by an O₂-pervious membrane.

Besides oxygen, generated by catalase from H₂O₂ there are other amperometrically detectable compounds, such as ferrocene derivatives or In²⁺salts. A novel approach is the use of the redox polymer PVP-Os(bipyridyl)₂Cl), which is coimmobilized with specific antibodies. Additionally, there are examples for enzymes with electrochemically active products. AP, for example, catalyzes the hydrolysis of phenyl phosphate or p-aminophenyl phosphate (4-APP) compounds, which result in electrochemically active phenol or p-aminophenol. Furthermore, enzymes, such as horseradish peroxidase (HRP), glucose oxidase, glucose-6-phosphate dehydrogenase, with subsequent amperometrical oxidation of NADH and others, have also been successfully applied as labels.

The main disadvantage for amperometric immunosensors of having an indirect sensing system, however, is compensated for by an excellent sensitivity. This is due to a linear analyte concentration range compared to a logarithmic relationship in potentiometric systems. Special attention must be directed to the system-inherent transport rate limitations for redox partners on the electrode surface.

Conductometric and Capacitive Immunosensors

These immunosensor transducers measure the alteration of the electrical conductivity in a solution at constant voltage, caused by biochemical enzymatic reactions which specifically generate or consume ions. The capacitance changes are measured using an electrochemical system, in which the bioactive element is immobilized onto a pair of noble metal, mostly Au or Pt, electrodes. There are only few clinical applications available, as the high ionic strength of biological matrices makes it difficult to record the relatively small net conductivity changes caused by the signaling reaction. To circumvent this problem, recently, an ion-channel conductance immunosensor, mimicking biological sensory functions, was developed. The basis of this technique is the fact that the conductance of a population of molecular ion channels, built of tethered gramicidin A and aligned across a lipid bilayer membrane, is changed by the antibody-antigen binding event. Different applications using various antibodies, linked to the ion-channel complex, are given.

Another approach is the measurement of changes of the surface conductivity. For example, a conductometric immunosensor for the determination of methamphetamine (MA) in urine was recently developed. Anti-MA antibodies were immobilized onto the surface of a pair of platinum electrodes. The immunocomplex formation caused a decrease in the conductivity between the electrodes. The measurement of the reciprocal capacitance, performed at alternating voltage, is advantageous compared to conductometric devices, and serves two purposes. The first is to test the insulating monolayer on the sensor noble metal surface. Self-assembling monolayers, have insulating properties. Besides this, they prevent the immunosensor from being affected by nonspecific binding phenomena. Even minor desorption of the monolayer results in an essential increase in capacitance. Thus, the actual quality of the device can be checked. The second application is the measurement of changes in the effective dielectric thickness of the insulating layer during antigen binding, when antibodies are linked to the alkylthiol layer. of course, this is on condition that the v-substitution of the alkylthiol monolayer does not compromise the insulation. Hence, a marked decrease of the electrical capacitance is observed and is used to quantitate the analyte. The destructive influence of lateral diffusion on nanostructured monolayers is prevented by use of the spreader-bar technique.

Optical Sensors

Optical immunosensors are most popular for bioanalysis and are today's largest group of transducers. This is due to the advantages of applying visible radiation compared to other transducer techniques. Additional benefits are the nondestructive operation mode and the rapid signal generation and reading. In particular, the introduction of fiber bundle optics (“optodes”) as optical waveguides and sophisticated optoelectronics offers increased versatility of these analytical devices for clinical applications.

Changes in adsorption, fluorescence, luminescence, scatter or refractive index (RI) occur when light is reflected at sensing surfaces. These informations are the physical basis for optical sensor techniques. Usually, applied detectors are photodiodes or photomultipliers.

There are numerous applications of either direct label-free optical detection of the immunological reaction, of labeled immunospecies, or of the products of enzymatic reactions. Most labels are fluorescent, but bio- and chemiluminescence species are also possible. It is worth mentioning that the label-free evanescence wave-related sensors explicitly represent an elegant methodology, which is a valuable alternative to sophisticated immunoassays. Nevertheless, label-free systems are prone to unsolved problems, such as nonspecific binding effects and poor analytical sensitivity to analytes with low molecular weight. Kubitschko et al. noted that despite the efforts, all immunosensors are still one magnitude less sensitive than commercial immunoassays for determining analytes in human serum, particularly those with low molecular weight. They claim the use of mass labels, such as latex particles, in order to enhance the signal. The authors demonstrated the optimization of a nanoparticle enhanced bidiffractive grating coupler immunosensor for the detection of thyroid-stimulating hormone (TSH, MW 28,000 Da). The excellent performance characteristics of this sensor clearly showed how future devices should work. The problem of unspecific binding, however, can also be controlled by applying a reference sensing region on the chip.

Total Internal Reflection Spectroscopy (TIRS)

The common principle of the following analytical devices is that in an optical sensor with two materials with different refractive indices (RI), total internal reflection occurs at a certain angle of the light beam being directed through the layer with the higher RI towards the sensing interface. By this, an evanescence wave is generated in the material with the lower RI. This wave, being an electrical vector of the wavelength of the incident light beam, penetrates further into the medium with exponentially attenuated amplitude. Biomolecules attached in that portion of the medium will interact inevitably with the evanescent wave and, therefore, lead to a distinctive diminution of the reflected light. The resolution is directly proportional to the length of interaction. Infrared spectroscopes, measuring attenuated total reflectance, are commonly built in the Kretschmann configuration: an optically absorbing film at the sensor's surface enables the measurement of the attenuated light intensity as a function of the wavelength of the incident beam. For total internal reflection fluorescence (TIRF), analytics benefit from the fact that incident light excites molecules with fluorescence characteristics near the sensor surface creating a fluorescent evanescent wave. The emerging fluorescence is finally detected. The technique has been developed mainly for an optical detection of fluorescence-labeled antibodies or antigens. In the latter case, the fluorescence capillary fill device (FCFD) technique is worth mentioning. The FCFD is designed by using a planar optical waveguide and a glass plate separated from each other by a capillary gap. Fluorophore-labeled antigen is attached on the surface of the glass plate, whereas antibodies are immobilized on the surface of the optical waveguide.

Another phenomenon, the optical diffraction, is used by the optical biosensor assay (OBA™) system: biomolecules are attached to the surface of a silanized wafer. The protein-coated surface is illuminated through a photo mask to create distinct periodic areas of active and inactive protein. Upon illumination with laser light, the diffraction grating caused by the ligand-binding process diffracts the incident light. An analyte-free negative sample does not result in diffraction because no antigen-antibody binding occurred creating the diffraction grating. The presence or absence of a diffraction signal differentiates between positive and negative samples. The intensity of the signal provides a quantitative measure of the analyte concentration.

Ellipsometry

If linearly polarized light of known orientation is reflected at oblique incidence from a surface, the reflected light is elliptically polarized. The shape and orientation of the ellipse depend on the angle of incidence, the direction of the polarization of the incident light, and the reflection properties of the surface. On adsorption of biomolecules onto a planar solid surface, phase and amplitude of the reflected light are altered and can be recorded by ellipsometric techniques. These changes in the polarization of the light are due to the alterations of the RI and the coating thickness. There are only few applications, such as the study of a cholera toxin-ganglioside GM1 receptor-ligand reaction, which were carried out using an ellipsometer.

Optical Dielectric Waveguides

Optical waveguides are glass, quartz or polymer films or fibers made of high RI material embedded between or in lower index dielectric materials. If a linearly polarized helium-neon laser light wave, introduced into the high index film or fiber, arrives at the boundary at an angle which is greater than the critical angle of total reflection, it is confined inside the waveguide. Similar to surface plasmon resonance, an evanescent field develops at the sensor's surface. In this case, however, the evanescent field is generated by the excitation of the light itself in the dielectric layer. Most of the laser light is transmitted into the device and multiple reflections occur as it travels through the medium if a bioactive substance is placed over the surface. Some of the light, however, penetrates the biolayer. This light is reflected back into the waveguide with a shift in phase interfering with the transmitted light. Thus, changes in properties of the biolayer can be followed by detecting the changes in interference.

Waveguides are often made in the form of fibers. These fiber-optic waveguide systems offer advantages for sensors when being used for hazardous analysis. Planar waveguide systems are also applicable for interferometers. They use laser light directed towards the surface of the waveguide with the attached biomolecules, which is subsequently split into two partial electrical (TE) and magnetic (TM) fieldwaves, perpendicular to each other. The interaction with the sample surface changes the relative phase between TE and TM by the different RI and surface thickness values. Various configurations, such as the Fabry-Perot monomode channel interferometer, the Mach-Zehnder interferometer or the related two-mode thin-film waveguide difference interferometer, have been successfully established.

Another technique uses thin corrugations etched into the surface of a waveguide. This grating coupler device allows the measurement of the coupling angle of either the input or output laser beam. Both beams are correlated to the RI within the evanescent field at the sensor's surface. Recently, a long-period grating fiber immunosensor has proven to be sensitive (enabling analyses down to the nanomolar range) and reproducible. Grating couplers are also used for optical waveguide lightmode spectroscopy (OWLS). The basic principle of the OWLS method is that linearly polarized light is coupled by a diffraction grating into the waveguide layer. The incoupling is a resonance phenomenon that occurs at a defined angle of incidence that depends on the RI of the medium covering the surface of the waveguide. In the waveguide layer, light is guided by total internal reflection to the edges where it is detected by photodiodes. By varying the angle of incidence of the light, the mode spectrum is obtained from which the effective RIs are calculated for both TE and TM.

Surface Plasmon Resonance (SPR)

Among the different detection systems, SPR is the most popular one. There are two leading systems on the market: the BIAcore™ systems from Biacore (Uppsala, Sweden) and the IAsys™ from Fisons Applied Sensor Technology (Cambridge, UK). Other systems with small market positions are the BIOS-1 from Artificial Sensing Instruments (Switzerland), the SPR-20 from Denki Kagaku Keiki (Japan), the SPEETA from Texas Instruments (USA), the IBIS from Windsor Scientific (UK) and the DPX from Quantech (USA). The first two commercial evanescence-wave devices are widespread in research laboratories due to the sophisticated apparatus and userfriendly control software. The BIAcore™, however, has the biggest market position.

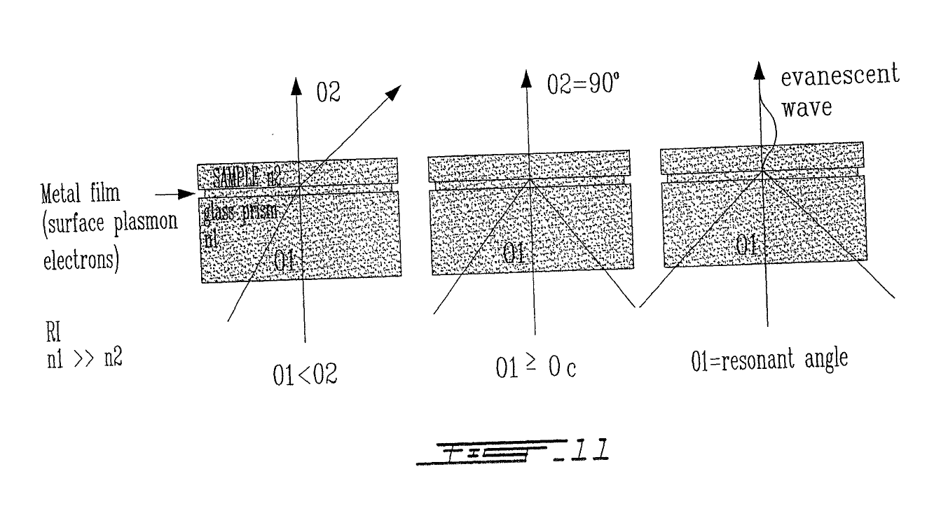

The general principle of SPR measurement 80 is depicted in FIG. 11. Polarized light is directed from a layer of high RI towards a layer with low RI to result in total internal reflection. The sample is attached to the layer of low RI. At the interface between the two different media, a thin approximately 50 nm gold film is interposed. Although light does not propagate into the low RI medium, the interfacial intensity is not equal to zero. The physical requirement of continuity across the interface is the reason for exciting the surface electrons “plasmons” in the metal film by the light energy. As a result, the electrons start oscillating. This produces an exponentially decaying evanescent wave penetrating a defined distance into the low RI medium, which is accountable for a characteristic decrease in the intensity of the reflected light. Hence, a direct insight in changes of the RI at the surface interface is made possible by monitoring the intensity and the resonance angle of the reflected light, caused by the biospecific interactions which took place there. Whereas in the BIAcore™ system, the light affects the sensing layer only once, there are several propagation contacts in the IASys™ due to the device's resonant mirror configuration. The BIAcore™ SPR apparatus is characterized by a sensitive measurement of changes of the RI when polarized laser light is reflected at the carboxy-methylated dextranactivated device interface. The IAsyS™ SPR device also uses a carboxy-methylated dextran-activated surface. Its dextran layer, however, is not attached to a gold surface, but to titanium, which forms a high refractive dielectric resonant layer. The glass prism is not attached tightly on the opposite side of the titanium layer, making space for an interposed silica layer of low RI. By this layer, the laser light beam couples into the resonant layer via the evanescent field. Therefore, the IAsys™ is seen as a combination of SPR resonant mirror with waveguide technology. As a result, no decrease in the reflected light intensity at resonance is observed in this system. The specific signal is the change in the phase of the reflected polarized light.

Differential SPR, a novel modification of a SPR immunosensor, improves further the sensitivity of the sensor by applying a modulation of the angle of light incidence. The reflectance curve is measured with a lock-in amplifier and recorded in the first and second derivative.

Light is directed from a prism with a RI towards a layer with low RI, resulting in total internal reflection. Although light does not propagate into the medium, the interfacial intensity is not equal to zero. Physical requirements of continuity across the interface are the cause of excitation of surface plasmons in the metal film by the light energy, causing them to oscillate. This produces an exponential evanescent decaying, which penetrates a defined distance into the low-index medium and results in a characteristic decrease in reflected light intensity.

Microgravimetric Sensors

A direct measurement of mass changes induced by the forming of antigen/antibody complexes is also enabled by acoustic sensors. The principle of operation is based on the propagation of acoustic shear waves in the substrate of the sensor. Phase and velocity of the acoustic wave are influenced by the specific adsorption of antibody molecules onto the antigen-coated sensor surface. Piezoelectric materials, such as quartz (SiO₂), zinc oxide (ZnO) or others resonate mechanically at a specific ultrasonic frequency in the order of tens of megahertz when excited in an oscillating electrical field. The resonant frequency is determined by the distance between the electrodes on both sides of the quartz plate, which is equal to the thickness of the plate and the velocity of the acoustic wave within the quartz material. In other words, electromagnetic energy is converted into acoustic energy, whereby piezoelectricity is associated with the electrical polarization of materials with anisotropic crystal structure. The most applied technique for monitoring the acoustic wave operation is the oscillation method. This means a configuration in which the device constitutes the frequency-controlling element of a circuit. The oscillation method measures the series resonant frequency of the resonating sensor.

The microgravimetric sensor devices are divided into quartz crystal microbalance (QCM) devices applying a thickness-shear mode (TSM), and devices applying a surface acoustic wave (SAW) detection principle. These sensors have reached considerable technical sophistication.

Additional bioanalytical application devices include the flexural plate wave (FPW), the shear horizontal acoustic plate (SH-APM), the surface transverse wave (STW) and the thin-rod acoustic wave (TRAW)

There are considerable similarities between the physical principles of QCM and SPR sensors, even when there are fundamental differences. Both QCM and SPR are wave-propagation phenomena and show resonance structure. The elastic QCM wave and the surface plasmon wave are nonradiative, i.e., an evanescent wave exists. Changes of physical properties within the evanescent field lead to a shift of resonance. Thus, a linear approximation of the physical relationship is allowed for immunological application in immunosensors.

The TSM Sensor

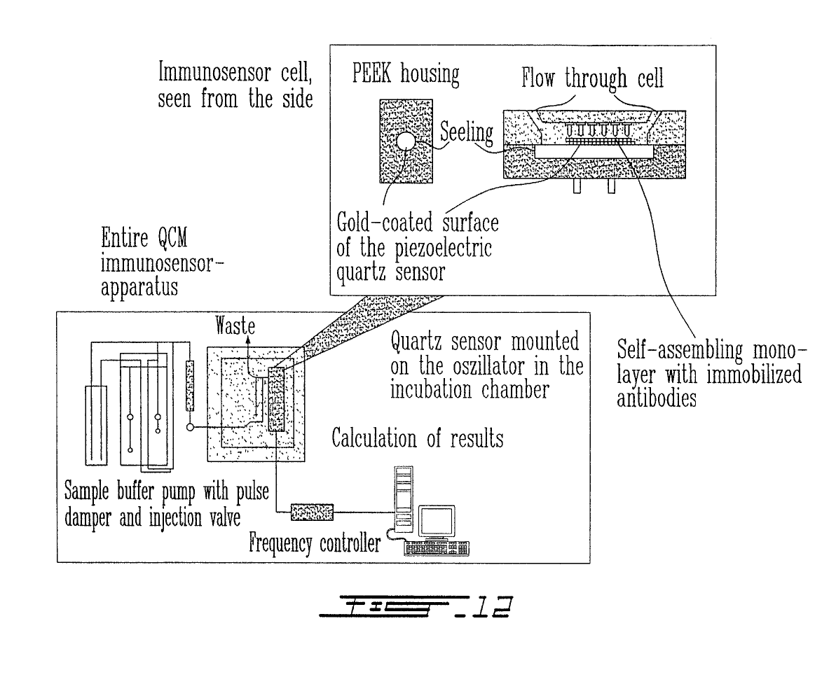

The TSM sensor consists of an AT-cut piezoelectric crystal disc, most commonly of quartz because of its chemical stability in biological fluids and resistance to extreme temperatures. The disc is attached to two metal electrodes on opposite sides for the application of the oscillating electric field. The TSM is run in a range of 5-20 MHz. The schematic design of a typical TSM device shown in FIG. 12. Advantages are, besides the chemical inertness, the low cost of the devices and the reliable quality of the mass-produced quartz discs. Major drawbacks of the system are the insensitivity for analytes with a molecular weight −1000 Da, and, as seen in all label-free immunosensor systems, nonspecific binding interferences. Nonspecific binding effects are hard to distinguish from authentic binding events due to the fact that no reference line can be placed in the sensor device. For a SH-APM device, however, by appropriately selecting the device frequency, these spurious responses can be suppressed. This sensor is applicable for measurements in human serum matrix.

One of the first applications of TSM technology was an immunosensor for human immunodeficiency virus (HIV) serology. This sensor was realized by immobilizing recombinant viral peptides on the surface of the transducer and by detecting anti-HIV antibodies directly in human sera.

The Saw Sensor

SAW sensors use thick ST-cut quartz discs and interdigitated metal electrode arrays that generate acoustic Rayleigh waves in both directions from the interdigital electrodes, their transmission being attenuated by surface-attached biomolecules. The oscillation frequency of a SAW sensor ranges from 30 to 500 MHz. The operation of SAW immunosensors with biological samples is compromised by the fact that the surface wave is considerably attenuated in the liquid phase. Thus, the domain of this technique is most likely restricted to gas phase operations.

The present invention is exemplified as an ELISA as described hereinbelow for corresponding probe substrate and or metabolites and the molar ratios thereof calculated to reveal the individual phenotypes.

TABLE 1
Examples of Enzymes and Corresponding Probes Drugs

| Enzyme | Probe substrate |
|---|---|
| NAT1 | p-aminosalicylic acid |
| NAT2 | Caffeine |
| CYP1A2 | Caffeine |
| CYP2A6 | Coumarin |
| CYP2C9 | s-Ibuprofen |
| CYP2C19 | Mephenytoin |
| CYP2D6 | Dextromethorphan |
| CYP2E1 | Chlorzoxazone |
| CYP3A4 | Midazolam |

In Example I, a detailed description of the synthesis of probe substrate and metabolite derivatives and the ELISA development for N-acetyltransferase(NAT2) are illustrated. The materials and methods, and the overall general process described for the development of the NAT2 ELISA method and kit for metabolic are adapted to the development of the metabolic phenotyping ELISA kits for other metabolic enzymes including NAT1, CYP1A2, CYP2A6, CYP2D6, CYP2E1, CYP3A4, CYP2C9 and CYP2C19, as well as a multi-determinant metabolic phenotyping system and method. In particular, the protocol as herein described for the development of an ELISA specific to NAT2 is adapted for the development of a CYP3A4-specific ELISA, in accordance with the present invention. Accordingly, an assay system is provided that is adapted for the characterization of phenotypic determinants of CYP3A4 and can be used for individualizing treatment with Alzheimer's disease agent. Furthermore, the present invention may also be adapted to provide for the identification of other characteristics or determinants of drug clearance and drug toxicity known to vary on an individual basis. EXAMPLE I

### Determination of Phenotypic Determinants by ELISA

NAT2

Different probe substrates can be used to determine the NAT2 phenotype (Kilbane, A. J. et al. (1990) Clin. Pharmacol. Ther., 47:470-477; Tang, B-K. et al. (1991) Clin. Pharmacol. Ther., 49:648-657). In accordance with the present invention caffeine is the preferred probe because it is widely consumed and relatively safe (Kalow, W. et al. (1993) Clin. Pharmacol. Ther., 53:503-514). In studies involving this probe, the phenotype has been generally determined from ratios of the caffeine metabolites 5-acetamino-6-amino-1-methyluracil (AAMU) or 5-acetamino-6-formylamino-1-methyluracil (AFMU) and 1-methylxanthine (1X). In these studies, the individuals are given an oral dose of a caffeine-containing substance, and the urinary concentrations of the target metabolites determined by HPLC (Kilbane, A. J. et al. (1990) Clin. Pharmacol. Ther., 47:470-477; Tang, B-K. et al. (1991) Clin. Pharmacol. Ther., 49:648-657) or CE (Lloyd, D. et al. (1992) J. Chrom., 578:283-291).

The number of clinical protocols requiring the determination of NAT2 phenotypes is rapidly increasing and in accordance with the present invention, an enzyme linked immunosorbent assay (ELISA) was developed for use in these studies (Wong, P., Leyland-Jones, B., and Wainer, I. W. (1995) J. Pharm. Biomed. Anal., 13:1079-1086). ELISAs have been successfully applied in the determination of low amounts of drugs and other antigenic components in plasma and urine samples, involve no extraction steps and are simple to carry out.

In accordance with the present invention, antibodies were raised in animals against two caffeine metabolites [5-acetamino-6-amino-1-methyluracil (AAMU) or 5-acetamino-6-formylamino-1-methyluracil (AFMU), and 1-methyl xanthine (1X)] present in urine samples of an individual collected after drinking coffee. Their ratio provides a determination of an individual's N-acetylation (NAT2) phenotype. Subsequently, there was developed a competitive antigen enzyme linked immunosorbent assay (ELISA) for measuring this ratio using these antibodies.

The antibodies of the present invention can be either polyclonal antibodies or monoclonal antibodies raised against two different metabolites of caffeine, which allow the measurement of the molar ratio of these metabolites.

In accordance with the present invention, the molar ratio of caffeine metabolites is used to determine the acetylation phenotype of the individual as follows. Individuals with a ratio less than 1.80 are slow acetylators.

Materials and Methods

Materials

Cyanomethylester, isobutyl chloroformate, dimethylsulfate, sodium methoxide, 95% pure, and tribu-tylamine were purchased from Aldrich (Milwaukee, Wis., USA); horse radish peroxidase was purchased from Boehringer Mannheim (Montreal, Que., Canada); Corning easy wash polystyrene microtiter plates were bought from Canlab (Montreal, Que., Canada); o-methylisourea hydrochloride was obtained from Lancaster Laboratories (Windham, N.H., USA); alkaline phosphatase conjugated to goat anti-rabbit IgGs was from Pierce Chemical Co. (Rockford, Ill., USA); bovine serum albumin fraction V initial fractionation by cold alcohol precipitation (BSA), complete and incomplete Freund's adjuvants, diethanolamine, 1-methylxanthine, p-nitrophenol phosphate disodium salt, o-phenylenediamine hydrochloride; porcine skin gelatin, rabbit serum albumin (RSA); Sephadex™ G25 fine, Tween™ 20 and ligands used for testing antibodies' cross reactivities were obtained from Sigma Chemical Co. (St. Louis, Mo., USA). Whatman™ DE52 diethylaminoethyl-cellulose was obtained from Chromatographic Specialties Inc. (Brockville, Ont., Canada). Dioxane was obtained from A&C American Chemicals Ltd. (Montreal, Que., Canada) and was refluxed over calcium hydride for 4 hours and distilled before use. Other reagents used were of analytical grade.

Synthetic Procedures

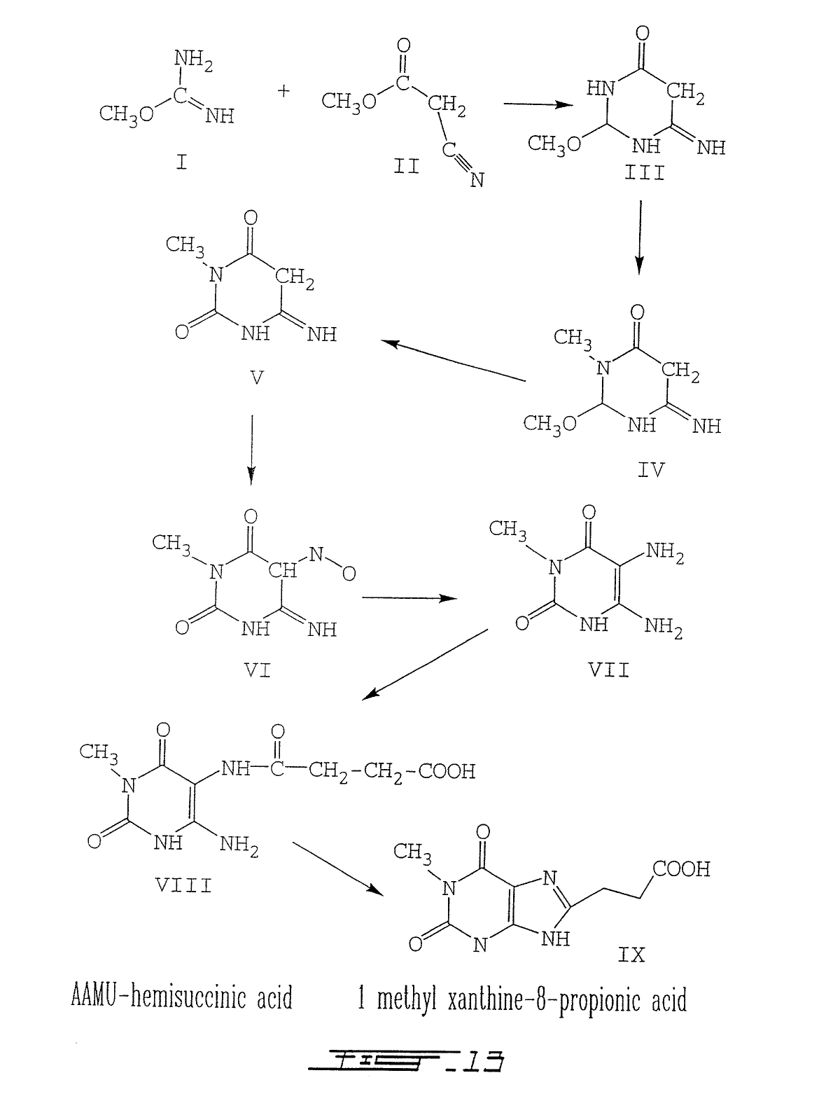

The synthetic route for the production of AAMU-hemisuccinic acid (VIII) and 1-methylxanthine-8-propionic acid (IX) is presented in FIG. 13.

Synthesis of 2-methoxy-4-imino-6-oxo-dihydropyridine (III)

Compound III is synthesized according to the procedure of Pfeiderer (Pfeilderer, W. (1957) Chem. Ber., 90:2272-2276) as follows. To a 250 mL round bottom flask 12.2 g of o-methylisourea hydrochloride (110.6 mmol), 11.81 mL methylcyanoacetate (134 mmol), 12.45 g of sodium methoxide (230.5 mmol) and 80 mL of methanol are added. The suspension is stirred and refluxed for 5 hours at 68-70° C. After cooling at room temperature, the suspension is filtered through a sintered glass funnel (Pyrex, 40-60 ASTM, 60 mL), and the NaCl on the filter is washed with methanol. The filtrate is filtered by gravity through a Whatman™ no. 1 paper in a 500 mL round bottom flask, and the solvent is evaporated under reduced pressure with a rotary evaporator at 50° C. The residue is solubilized with warm distilled water, and the product is precipitated by acidification to pH 3-4 with glacial acetic acid. After 2 hours (or overnight) at room temperature, the suspension is filtered under vacuum through a sintered glass funnel (Pyrex, 40-60 ASTM, 60 mL). The product is washed with water, acetone, and dried. The product is recrystallized with water as the solvent and using charcoal for decolorizing (activated carbon, Norit^{r} A<100 mesh, decolorizing). The yield is 76%.

Synthesis of 1-methyl-2-methoxy-4-imino-6-oxo-dyhydrooyrimidinr (IV)

Compound IV is synthesized according to the procedure of Pfeiderer (Pfeilderer, W. (1957) Chem. Ber., 90:2272-2276) as follows. To a 250 mL round bottom flask 11 g of compound III (77.0 mmol) and 117 mL of 1N NaOH (freshly prepared) are added. The solution is stirred and cooled at 15° C., using a water bath and crushed ice. Then 11.7 mL dimethylsulfate (123.6 mmol) are added dropwise with a pasteur pipette over a period of 60 min. Precipitation eventually occurs upon the addition. The suspension is stirred at 15° C. for 3 hours and is left at 4° C. overnight. The product is recovered by filtration under vacuum through a sintered glass funnel (Pyrex, 40-60 ASTM, 60 mL). The yield is 38%.

Synthesis of 1-methyl-4-iminouracil (V)

Compound V is synthesized according to the procedure of Pfeiderer (Pfeilderer, W. (1957) Chem. Ber., 90:2272-2276) as follows. To a 250 mL round bottom flask 11.26 g of compound IV (72.6 mmol) and 138 mL 12 N HCl are added, and the suspension is stirred at room temperature for 16-20 hours. The suspension is cooled on crushed ice, the product is recovered by filtration under vacuum through a sintered glass funnel (Pyrex, 40-60 ASTM, 60 mL). The product is washed with water at 4° C., using a pasteur pipette, until the pH of filtrate is around 4 (about 150 mL). The product is washed with acetone and dried. The yield is 73%.

Synthesis of 1-methyl-4-imino-5-nitrouracil (VI)

Compound VI is synthesized according to the procedure of Lespagnol et al (Lespagnol, A. et al. (1970) Chim. Ther., 5:321-326) as follows. To a 250 mL round bottom flask 6.5 g of compound V (46 mmol) and 70 mL of water are added. The suspension is stirred and refluxed at 100° C. A solution of 6.5 g sodium nitrite (93.6 mmol) dissolved in 10 mL water is added gradually to the reaction mixture with a pasteur pipette. Then 48 mL of glacial acetic acid is added with a pasteur pipette. Upon addition, precipitation occurs and the suspension becomes purple. The suspension is stirred and heated for an additional 5 min., and cooled at room temperature and then on crushed ice. The product is recovered by filtration under vacuum through a sintered glass funnel (Pyrex, 10-15 ASTM, 60 mL). It is washed with water at 4° C. to remove acetic acid and then with acetone. Last traces of acetic acid and acetone are removed under a high vacuum. The yield is 59%.

Synthesis of 1-methyl-4,5-diaminouracil (VII)

Compound VII is synthesized by the procedure of Lespagnol et al. (Lespagnol, A. et al. (1970) Chim. Ther., 5:321-326) as follows. To a 100 mL round bottom flask 2 g of compound VI (11.7 mmol) and 25 mL water are added. The suspension is stirred and heated in an oil bath at 60° C. Sodium hydrosulfite (88%) is gradually added (40.4 mmol), using a spatula, until the purple color disappears (approximately 5 g or 24.3 mmol). The suspension is heated for an additional 15 min. The suspension is cooled on crushed ice and left at 4° C. overnight. The product is recovered by filtration under vacuum through a sintered glass funnel (Pyrex, 30-40 ASTM, 15 mL). The product is washed with water and acetone, and dried. The last traces of acetone are removed under a high vacuum. The yield is 59%.

Synthesis of AAMU-hemisuccinic Acid (VIII)

Compound VIII is synthesized as follows. To a 20 mL beaker 0.30 g of compound VII (1.92 mmol) and 5 mL water are added. The suspension is stirred and the pH is adjusted between 8 to 9 with a 3N NaOH solution. Then 0.33 g succinic anhydride (3.3 mmol) is added to the resulting solution, and the mixture is stirred until the succinic anhydride is dissolved. During this process, the pH of the solution is maintained between 8 and 9. The reaction is completed when all the succinic anhydride is dissolved and the pH remains above 8. The hemisuccinate is precipitated by acidification to pH 0.5 with 12N HCl. The product is recovered by filtration on a Whatman™ No. 1 paper, and washed with water to remove HCl. It is then washed with acetone and dried.

Other AAMU or AFMU Derivates

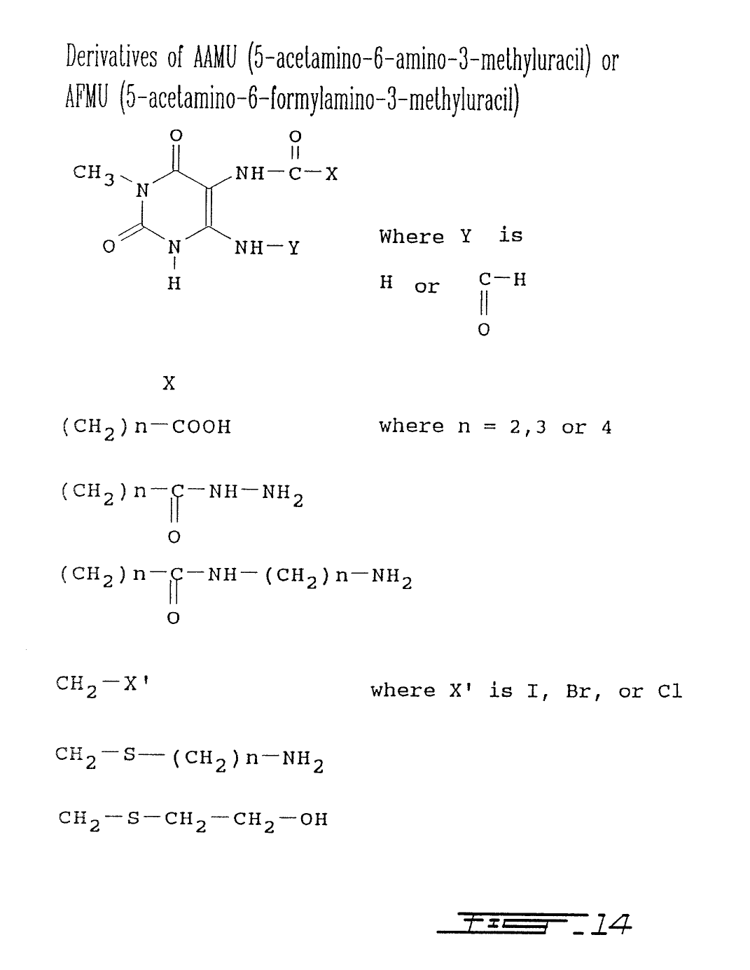

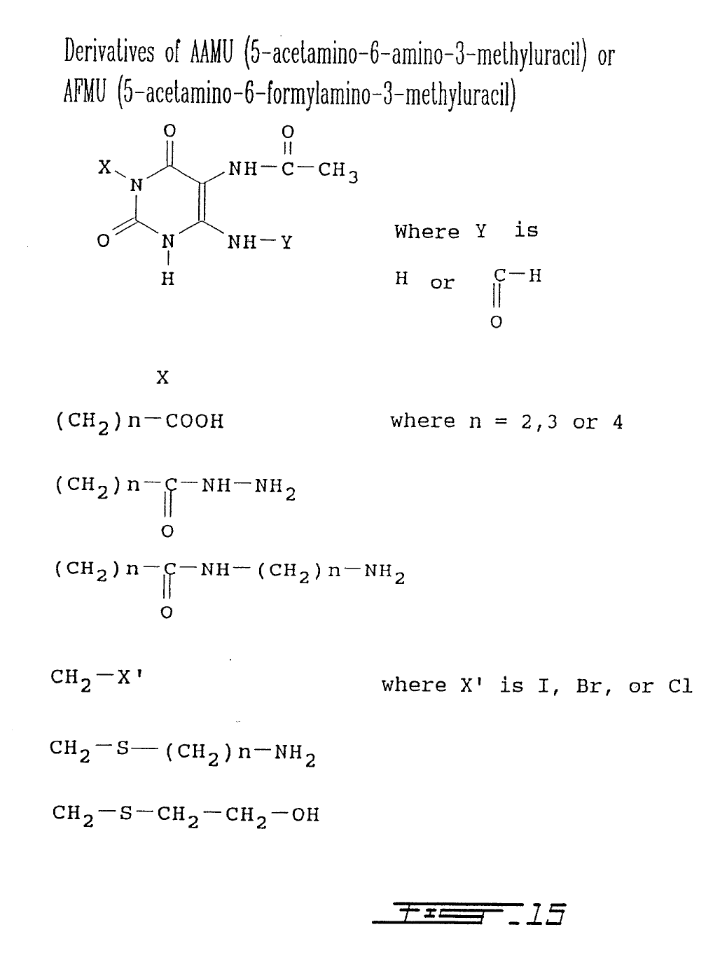

The derivatives shown in FIGS. 14 and 15 can also be used for raising antibodies against AAMU or AFMU that can be used for measuring the concentrations of these caffeine metabolites in urine samples.

Synthesis of 1-methylxanthine-8-propionic Acid (IX)

This product is synthesized according to a modified procedure of Lespagnol et al. (Lespagnol, A. et al. (1970) Chim. Ther., 5:321-326) as follows. A 0.2 g sample of compound VIII (0.78 mmol) is dissolved in 2-3 mL of a 15% NaOH solution. The resulting solution is stirred at 100° C. until all of the solvent is evaporated, and is then maintained at this temperature for an additional 5 min. The resulting solid is cooled at room temperature, and dissolved in 10 mL water. The product is precipitated by acidification to pH 2.8 with 12 N HCl. After cooling at 4° C. for 2.5 hours, the product is recovered by filtration on a Whatman™ No. 1 paper, washed with water and acetone, and dried. It is recrystallized from water-methanol (20:80, v/v), using charcoal to decolorize the solution.

Other Derivatives of 1X

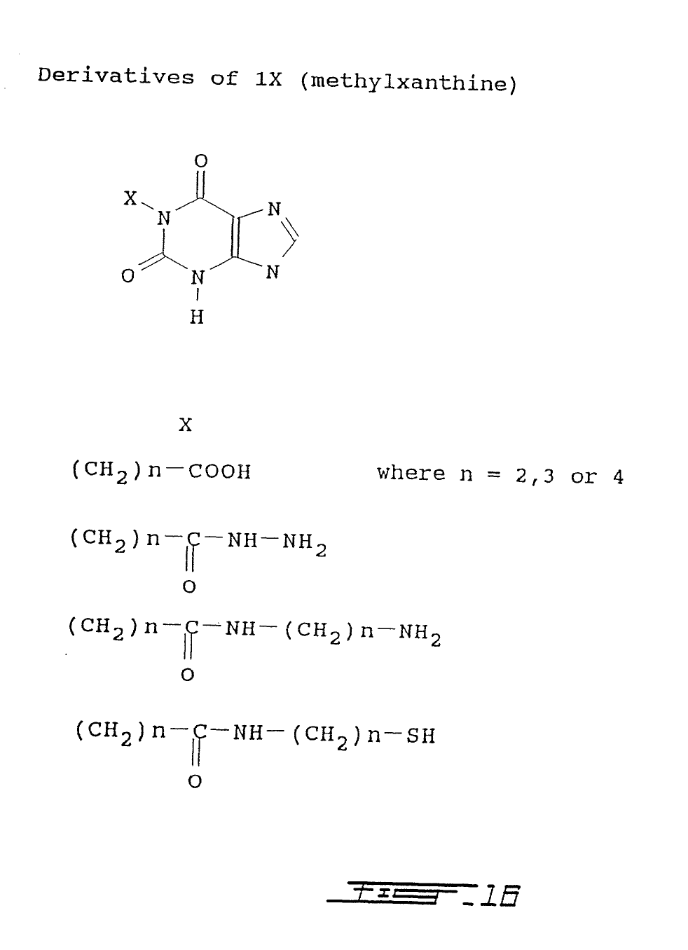

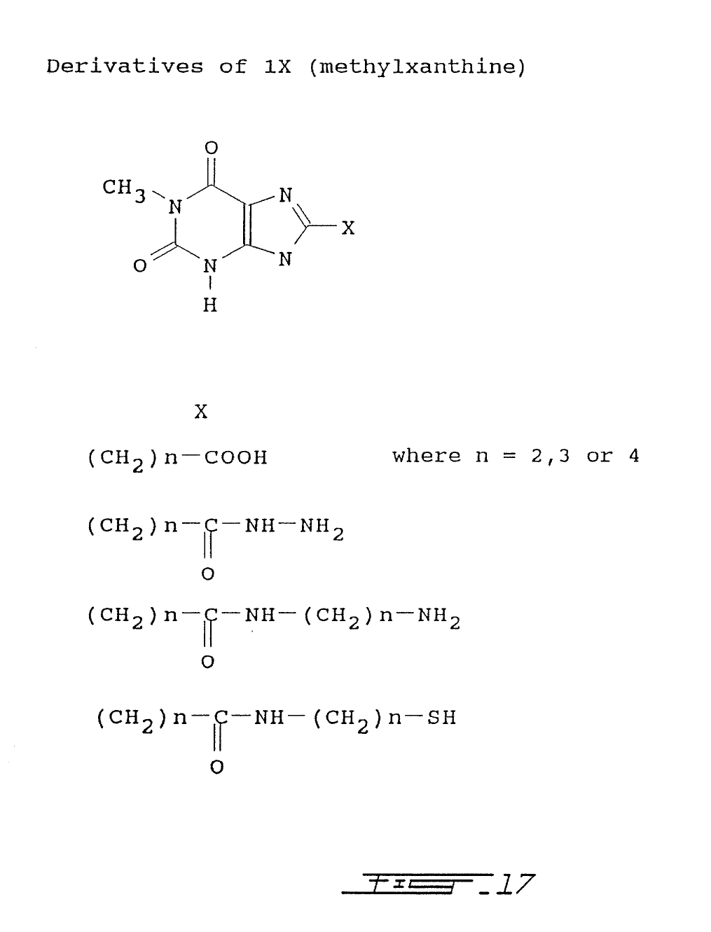

The other derivatives of 1X, shown in FIGS. 16 and 17, can also be used for raising antibodies against 1X and thereby to allow the development of an ELISA for measuring 1X concentration in urine samples.

Synthesis of AAMU

AAMU is synthesized from compound VII according to the procedure of Fink et al (Fink, K. et al. (1964) J. Biol. Chem., 249:4250-4256) as follows. To a 100 mL round bottom flask 1.08 g of compound VII (6.9 mmol) and 20 mL acetic acid anhydride were added. The suspension is stirred and refluxed a 160-165° C. for 6 min. After cooling at room temperature, the suspension is filtered under vacuum through a sintered glass funnel (Pyrex, 10-15 ASTM, 15 mL). The product is washed with water and acetone, and dried. The product is recrystallized in water.

NMR Spectroscopy

¹H and ¹³C NMR spectra of compounds VIII and IX are obtained using a 500 MHz spectrophotometer (Varian™ XL 500 MHz, Varian Analytical Instruments, San Fernando, Calif., USA) using deuterated dimethyl sulfoxide as solvent.

Conjugation of Haptens to Bovine Serum Albumin and Rabbit Serum Albumin

The AAMU-hemisuccinic acid (VIII) and the 1-methylxanthine propionic acid (IX) are conjugated to BSA and RSA according to the following mixed anhydride method. To a 5 mL round bottom flask 31.7 mg of compound VIII (0.12 mmol) or 14.9 mg of compound IX (0.06 mmol) are added. Then 52.2 μL of tri-n-butylamine (0.24 mmol) and 900 μL of dioxane, dried over calcium hydride and freshly distilled, are added. The solution is cooled at 10° C. in a water bath using crushed ice. Then 12.6 μL isobutyl chloroformate at 4° C. (0.12 mmol, recently purchased or opened) are added and the solution is stirred for 30-40 min at 10-12° C. While the above solution is stirring, a second solution is prepared as follows. In a glass tube 70 mg BSA or RSA (0.001 mmol) are dissolved in 1.83 mL water. Then 1.23 mL dioxane, freshly dried and distilled, is added and the BSA or RSA solution is cooled on ice. After 30-40 min of the above stirring, 70 μL of 1 N NaOH solution cooled on ice is added to the BSA or RSA solution and the resulting solution is poured in one portion to the flask containing the first solution. The solution is stirred at 10-12° C. for 3 hours and dialyzed against 1 liter of water for 2 days at room temperature, with water changed twice a day. The protein concentration of the conjugates and the amounts of moles of AAMU or 1X incorporated per mole of BSA or RSA is determined by methods described below. The products are stored as 1 mL aliquots at −20° C.

Protein Determination by the Method of Lowry et al. (Lowry, O. H. et al. (1951) J. Biol. Chem., 193:265-275)

| A) SOLUTIONS | |
|---|---|
| Solution A: | 2 g Na₂CO₃ is dissolved in 50 mL water, 10 |
| | mL of 10% SDS and 10 mL 1 NaOH, water is |
| | added to 100 mL. Freshly prepared. |
| Solution B: | 1% NaK Tartrate |
| Solution C: | 1% CuSO₄.5H₂O |
| Solution D: | 1 N phenol (freshly prepared): 3 mL Folin & |
| | Ciocalteu's phenol reagent (2.0 N) and 3 mL |
| | water. |
| Solution F: | 98 mL Solution A, 1 mL Solution B, 1 mL |
| | Solution C. Freshly prepared. |
| BSA: | 1 mg/mL. 0.10 g bovine serum albumin |
| | (fraction V)/100 mL. |

B) ASSAY

| Standard curve | Tubes # (13 × 100 mm) | | | | | | |
|---|---|---|---|---|---|---|---|
| Solution | 1 | 2 | 3 | 4 | 5 | 6 | 7 |
| BSA (μL) | 0 | 10 | 15 | 20 | 30 | 40 | 50 |
| Water (μL) | 200 | 190 | 185 | 180 | 170 | 160 | 150 |
| Solution F (mL) | 2.0 | 2.0 | 2.0 | 2.0 | 2.0 | 2.0 | 2.0 |

The solutions are vortexed and left 10 min at room temperature

| | | | | | | | |
|---|---|---|---|---|---|---|---|
| Solution D (μL) | 200 | 200 | 200 | 200 | 200 | 200 | 200 |

The solutions are vortexed and left at room temperature for 1 hour.

The absorbance of each solution is read at 750 nm using water as the blank.

UNKNOWN

| | Tube # (13 × 100 mm) | | |
|---|---|---|---|
| Solution D.F.^{a} | 1 | 2 | 3 |
| Unknown (μL) | x | x | x |
| Water (μL) | y | y | y |
| | x + y = 200 μL | | |
| Solution F (mL) | 2.0 | 2.0 | 2.0 |
| Solution D (μL) | 200 | 200 | 200 |

The solutions are vortexed and left 10 min at room temperature.

The solutions are vortexed and left 1 hour at room temperature.

The absorbance of each solution is read at 750 nm using water as the blank.

The protein concentration is calculated using the standard curve and taking account of the dilution factor (D.F.).

a. D.F. (dilution factor). It has to be such so that the absorbance of the unknown at 750 nm is within the range of absorbance of the standards.

Method to Determine the Amounts of Moles of AAMU or 1X Incorporated per Mole of BSA OR RSA.

This method gives an approximate estimate. It is a useful one because it allows one to determine whether the coupling proceeded as expected.

A) Solutions

10% sodium dodecyl sulfate (SDS)

1% SDS solution

0.5 or 1 mg/mL of AAMU-BSA (or AAMU-RSA) in a 1% SDS solution (1 mL).

0.5 or 1 mg/mL of BSA or RSA in a 1% SDS solution (1 mL).

B) Procedure

The absorbance of the AAMU conjugate solution is measured at 265 nm, with 1% SDS solution as the blank.

The absorbance of the BSA (or RSA) solution is measured at 265 nm, with 1% SDS solution as the blank.

The amount of moles of AAMU incorporated per mole of BSA (or RSA) is calculated with this formula:

$$y = \frac{{A}_{265} ( AAMU \text{-} BSA ) - {A}_{265} ( BSA )}{{ϵ}_{265} ( AAMU ) \times [ BSA ]}$$

Where:

y is the amount of moles of AAMU/mole of BSA (or RSA);

ε₂₆₅ (AAMU) is the extinction coefficient of AAMU=10⁴ M⁻¹cm⁻¹; and

[BSA]=BSA (mg/mL)/68,000/mmole.

To calculate the amount of moles of 1X incorporated per mole of BSA or RSA, the same procedure is used but with this formula:

$$y = \frac{{A}_{252} ( 1 X \text{-} BSA ) - {A}_{252} ( BSA )}{{ϵ}_{252} ( 1 X ) \times [ BSA ]}$$

Where:

y is the amount of moles of 1X/mole of BSA (or RSA);

ε₂₅₂ (AAMU) is the extinction coefficient of 1×=10⁴ M⁻¹cm⁻¹; and

[BSA]=BSA (mg/mL)/68,000/mmole.

Coupling of Haptens to Horse Radish Peroxidase

The AAMU derivative (VIII) and 1X derivative (IX) are conjugated to horse radish peroxidase (HRP) by the following procedure. To a 5 mL round bottom flask 31.2 mg of compound VIII (or 28.3 mg of compound IX) are added. Then 500 μL of dioxane, freshly dried over calcium chloride, are added. The suspension is stirred and cooled at 10° C. using a water bath and crushed ice. Then 114 μL tributylamine and 31 μL of isobutyl chloroformate (recently opened or purchased) are added. The suspension is stirred for 30 min at 10° C. While the suspension is stirring, a solution is prepared by dissolving 13 mg of horse radish peroxidase (HRP) in 2 mL of water. The solution is cooled at 4° C. on crushed ice. After the 30 min stirring, 100 μL of a 1 N NaOH solution at 4° C. is added to the HRP solution and the alkaline HRP solution is poured at once into the 5 mL flask. The suspension is stirred for 4 hours at 10-12° C. The free derivative is separated from the HRP conjugate by filtration through a Sephadex G-25™ column (1.6×30 cm) equilibrated and eluted with a 0.05 M sodium phosphate buffer, pH 7.5. The fractions of 1.0-1.2 mL are collected with a fraction collector. During the elution two bands are observed: the HRP conjugate band and a light yellow band behind the HRP conjugate band. The HRP conjugate elutes between fractions 11-16. The fractions containing the HRP conjugate are pooled in a 15 mL tissue culture tube with a screw cap. The HRP conjugate concentration is determined at 403 nm after diluting an aliquot (usually 50 μL+650 μL of buffer).

[HRP-conjugate](mg/mL)=A₄₀₃×0.4×D.F.

The ultraviolet (UV) absorption spectrum is recorded between 320 and 220 nm. The presence of peaks at 264 and 270 nm for AAMU-HRP and 1X-HRP conjugates, respectively, are indicative that the couplings proceeded as expected.

After the above measurements, 5 μL of a 4% thiomersal solution is added per mL of the AAMU-HRP or 1X-HRP conjugate solution. The conjugates are stored at 4° C.

Antibody Production

Four mature females New Zealand white rabbits (Charles River Canada, St-Constant, Que., Canada) are used for antibody production. The protocol employed in this study was approved by the McGill University Animal Care Committee in accordance with the guidelines from the Canadian Council on Animal Care. Antibodies of the present invention may be monoclonal or polyclonal antibodies.

An isotonic saline solution (0.6 mL) containing 240 mg of BSA conjugated antigen is emulsified with 0.6 mL of a complete Freund's adjuvant. A 0.5 mL aliquot of the emulsion (100 mg of antigen) is injected per rabbit intramuscularly or subcutaneously. Rabbits are subsequently boosted at intervals of three weeks with 50 mg of antigen emulsified in incomplete Freund's adjuvant. Blood is collected by venipuncture of the ear 10-14 days after boosting. Antisera are stored at 4° C. in the presence of 0.01% sodium azide.

Double Immunidiffusion in Agar Plate

An 0.8% agar gel in PBS is prepared in a 60×15 mm petri dish. Rabbit serum albumin (100 μL of 1 mg mL⁻¹) conjugated to AAMU (or 1X) are added to the center well, and 100 μL of rabbit antiserum are added to the peripheral wells. The immunodiffusion is carried out in a humidified chamber at 37° C. overnight and the gel is inspected visually.

Antiserum Titers

The wells of a microtiter plate are coated with 10 μg mL⁻¹ of rabbit serum albumin-AAMU (or 1X) conjugate in sodium carbonate buffer, pH 9.6) for 1 hour at 37° C. (100 μL/per well). The wells are then washed three times with 100 μL TPBS (phosphate buffer saline containing 0.05% Tween™ 20) and unoccupied sites are blocked by an incubation with 100 mL of TPBS containing 0.05% gelatin for 1 hour at 37° C. The wells are washed three times with 100 μL TPBS and 100 μL of antiserum diluted in TPBS are added. After 1 hour at 37° C., the wells are washed three times with TPBS, and 100 μL of goat anti-rabbit IgGs-alkaline phosphatase conjugate, diluted in PBS containing 1% BSA, are added. After 1 hour at 37° C., the wells are washed three times with TPBS and three times with water. To the wells are added 100 μL of a solution containing MgCl₂ (0.5 mM) and p-nitrophenol phosphate (3.85 mM) in diethanolamine buffer (10 mM, pH 9.8). After 30 min. at room temperature, the absorbency is read at 405 nm with a microplate reader. The antibody titer is defined as the dilution required to change the absorbance by one unit (1 au).

Isolation of Rabbit IgGS

The DE52-cellulose resin is washed three times with sodium phosphate buffer (500 mM, pH 7.50), the fines are removed and the resin is equilibrated with a sodium phosphate buffer (10 mM, pH 7.50). The resin is packed in a 50×1.6 cm column and eluted with 200-300 mL equilibrating buffer before use. To antiserum obtained from 50 mL of blood (30-32 mL) is added dropwise 25-27 mL of a 100% saturated ammonium sulfate solution with a Pasteur pipette. The suspension is left at room temperature for 3 h and centrifuged for 30 min. at 2560 g at 20° C. The pellet is dissolved with 15 mL sodium phosphate buffer (10 mM, pH 7.50) and dialyzed at room temperature with the buffer changed twice per day. The dialyzed solution is centrifuged at 2560 g for 10 min. at 20° C. to remove precipitate formed during dialysis. The supernatant is applied to the ion-exchange column. Fractions of 7 mL are collected. After application, the column is eluted with the equilibrating buffer until the absorbance at 280 nm becomes less than 0.05 au. The column is then eluted with the equilibrating buffer containing 50 mM NaCl. Fractions having absorbencies greater than 0.2 at 280 nm are saved and stored at 4° C. Protein concentrations of the fractions are determined as described above.

Competitive Antigen ELISA

Buffers and water without additives are filtered through millipore filters and kept for 1 week. BSA, antibodies, Tween™ 20 and horse radish peroxidase conjugates are added to these buffers and water just prior to use. Urine samples are usually collected 4 hours after drinking a cup of coffee (instant or brewed with approximately 100 mg of caffeine per cup) and stored at −80° C. The urine samples are diluted 10 times with sodium phosphate buffer (620 mosm, pH 7.50) and are subsequently diluted with water to give concentrations of AAMU and 1X no higher than 3×10⁻⁶ M in the ELISA. All the pipettings are done with an eight-channel pipette, except those of the antibody and sample solutions. Starting with the last well, 100 μL of a carbonate buffer (100 mM, pH 9.6) containing 2.5 μg mL⁻¹ antibodies are added to each well. After 90 min. at room temperature, the wells are washed three times with 100 mL of TPB: isotonic sodium phosphate buffer (310 mosm, pH 7.50) containing 0.05% Tween™ 20.

After the initial wash, unoccupied sites are blocked by incubation for 90 min. at room temperature with 100 μL TBP containing 3% BSA. The wells are washed four times with 100 μL TPB. The washing is followed by additions of 50 μL of 12 mg mL⁻¹ AAMU-HRP or 1X-HRP conjugate in 2×TPB containing 2% BSA, and 50 μL of either water, standard (13 standards; AAMU or 1X , 2×10⁻⁴ to 2×10⁻⁸ M) or sample in duplicate. The microplate is gently shaken with an orbital shaker at room temperature for 3-4 hours. The wells are washed three times with 100 μL TPB containing 1% BSA and three times with water containing 0.05% Tween™ 20. To the washed plate is added 150 μL of a substrate buffer composed of citric acid (25 mM) and sodium phosphate dibasic buffer (50 mM, pH 5.0) containing 0.06% hydrogen peroxide and 0.04% o-phenylenediamine hydrochloride. After 20 min. at room temperature with shaking, the reaction is stopped with 50 μL of 2.5 M HCl. After shaking the plate 3 min., the absorbances are read with a microtiter plate reader at 490 nm.

Results

Polyclonal antibodies against AAMU and 1X could be successfully raised in rabbits after their conjugation to bovine serum albumin. Each rabbit produced antibody titers of 30,000-100,000 as determined by ELISA. This was also indicated by strong precipitation lines after double immunodiffusion in agar plates of antisera and derivatives conjugated to rabbit serum albumin. On this basis, a) IgGs antibodies were isolated on a DE-52 cellulose column and b) a competitive antigen ELISA for NAT2 phenotyping using caffeine as probe substrate was developed according to the methods described in the above section entitled Materials and Methods.

Contrary to current methods used for phenotyping, the assay involves no extraction, is sensitive and rapid, and can be readily carried out on a routine basis by a technician with a minimum of training in a clinical laboratory.

The present invention will be more readily understood by referring to the following examples which are given to illustrate the invention rather than to limit its scope.

A Competitive Antigen ELISA for NAT2 Phenotyping Using Caffeine as a Probe Substrate

Buffers and water without additives were filtered through millipore filters and kept for 1 week. BSA, antibodies, Tween™ 20 and horse radish peroxidase conjugates were added to these buffers and water just prior to use. Urine samples were usually collected 4 hours after drinking a cup of coffee (instant or brewed with approximately 100 mg of caffeine per cup) and stored at −80° C. They were diluted 10 times with sodium phosphate buffer (620 mosm, pH 7.50) and were subsequently diluted with water to give concentrations of AAMU and 1X no higher than 3×10⁻⁶ M in the ELISA. All the pipettings were done with an eight-channel pipette, except those of the antibody and sample solutions. Starting with the last well, 100 μL of a carbonate buffer (100 mM, pH 9.6) containing 2.5 μg mL⁻¹ antibodies was pipetted. After 90 min. at room temperature, the wells were washed three times with 100 μL of TPB: isotonic sodium phosphate buffer (310 mosm, pH 7.50) containing 0.05% Tween™ 20.

After the initial wash, unoccupied sites were blocked by incubation for 90 min. at room temperature with 100 μL TBP containing 3% BSA. The wells were washed four times with 100 μL TPB. This was followed by additions of 50 μL of 12 mg mL⁻¹ AAMU-HRP or 1X-HRP conjugate in 2×TPB containing 2% BSA, and 50 μL of either water, standard (13 standards; AAMU or 1X, 2×10⁻⁴ to 2×10⁻⁸ M) or sample in duplicate. The microplate was gently shaken with an orbital shaker at room temperature for 3-4 hours. The wells were washed three times with 100 μL with TPB containing 1% BSA and three times with water containing 0.05% Tween™ 20. To the washed plate was added 150 μL of a substrate buffer composed of citric acid (25 mM) and sodium phosphate dibasic buffer (50 mM, pH 5.0) containing 0.06% hydrogen peroxide and 0.04% o-phenylenediamine hydrochloride. After 20 min. at room temperature with shaking, the reaction was stopped with 50 μL of 2.5 N HCl. After shaking the plate 3 min., the absorbances were read with a microtiter plate reader at 490 nm.

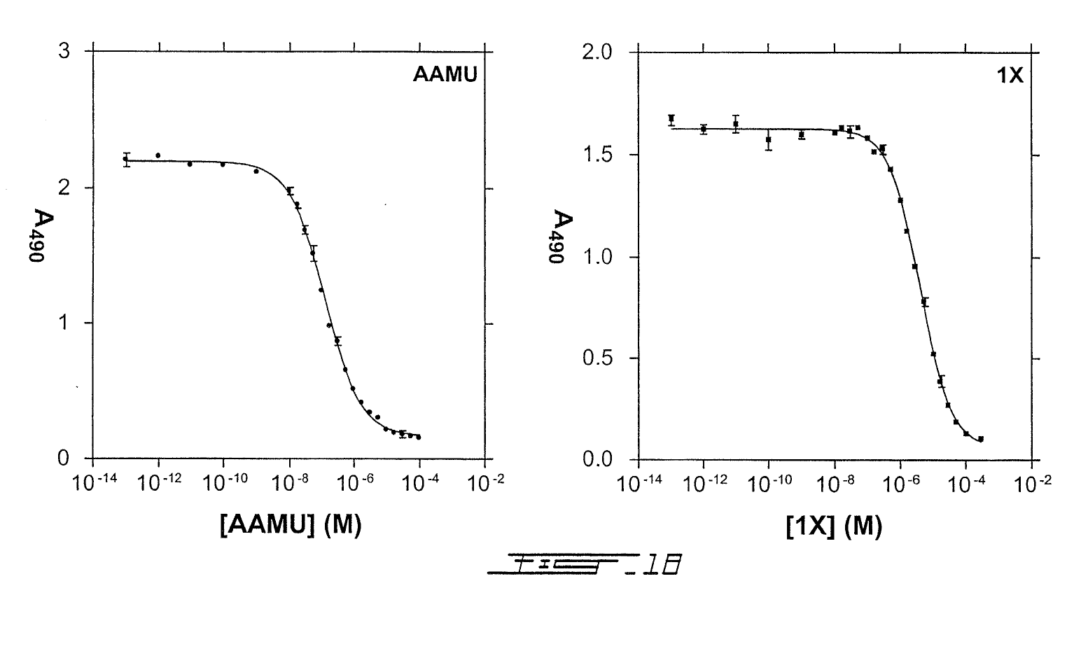

The competitive antigen ELISA curves of AAMU-Ab and 1X-Ab determinations obtained in duplicate are presented in FIG. 18. Each calibration curve represents the average of two calibration curves. The height of the bars measure the deviations of the absorbency values between the two calibration curves. Data points without bars indicate that deviations of the absorbency values are equal or less than the size of the symbols representing the data points. Under the experimental conditions of the ELISA: background was less than 0.10 au; the practical limits of detection of AAMU and 1X were 2×10⁻⁷ M and 2×10⁻⁶ M, respectively, concentrations 500 and 50 times lower than those in urine samples from previous phenotyping studies (Kilbane, A. J. et al. (1990) Clin. Pharmacol. Ther., 47:470-477); the intra-assay and interassay coefficients of variations of AAMU and 1X were 15-20% over the concentration range of 0.01-0.05 mM.

A variety of conditions for the ELISA were tested and a number of noteworthy observations were made: gelatin, which was used in the competitive antigen ELISA determination of caffeine in plasma (Fickling, S. A. et al. (1990) J. Immunol. Meth., 129:159-164), could not be used in our ELISA owing to excessive background absorbency which varied between 0.5 and 1.0 au; in the absence of Tween™ 20, absorbency changes per 15 min. decreased by a factor of at least 3, and calibration curves were generally erratic; absorbency coefficients of variation of samples increased by a factor of 3 to 4 when the conjugates and haptens were added to the wells as a mixture instead individually.

The cross reactivities of AAMU-Ab and 1X-Ab were tested using a wide variety of caffeine metabolites and structural analogs (Table 2 below). AAMU-Ab appeared highly specific for binding AAMU, while 1X-Ab appeared relatively specific for binding 1X. However, a 11% cross reactivity was observed with 1-methyluric acid (1U), a major caffeine metabolite.

TABLE 2
Cross-reactivity of AAMU-Ab and 1X-Ab towards different
caffeine metabolites and structural analogs

| | % Cross-Reaction | |
|---|---|---|
| Compound | AAMU-Ab | 1X-Ab |
| Xanthine | 0^{a} | 0 |
| Hypoxanthine | 0 | 0 |
| 1-Methyl Xanthine (1X) | 0 | 100 |
| 3-Methyl Xanthine | 0 | 0 |
| 7-Methyl Xanthine | 0 | 0 |
| 8-Methyl Xanthine | 0 | 0 |
| 1,3-Dimethyl Xanthine (Theophylline) | 0 | 0.2 |
| 1,7-Dimethyl Xanthine (Paraxanthine) | 0 | 0.5 |
| 3,7-Dimethyl Xanthine (Theobromine) | 0 | 0 |
| 1,3,7-Trimethyl Xanthine (Caffeine) | 0 | 0 |
| Uric acid | 0 | 0 |
| 1-Methyluric acid | 0 | 11 |
| 1,7-Dimethyluric acid | 0 | 0 |
| Guanine | 0 | 0 |
| Uracil | 0 | 0 |
| 5-Acetamino-6-amino-uracil | 0.6 | 0 |
| 5-Acetamino-6-amino-1-methyluracil (AAMU) | 100 | 0 |
| 5-Acetamino-6-amino-1,3-dimethyluracil | 0 | 0 |

a. The number 0 indicates either an absence of inhibition or an inhibition no higher than 40% at the highest compound concentration tested in the ELISA (5×10⁻³ M); concentrations of 5-acetamino-6-amino-1-methyluracil (AAMU) and 1-Methyl Xanthine (1X) required for 50% inhibition in the competitive antigen ELISA were 1.5×10⁻⁶ M and 10⁻⁵ M, respectively.

The relative high level of cross reactivity of 1U is, however, unlikely to interfere significantly in the determination of 1X and the assignment of NAT2 phenotypes, since the ratio of 1U:1X is no greater than 2.5:1 in 97% of the population (Tang, B-K. et al. (1991) Clin. Pharmacol. Ther., 49:648-657). This is confirmed by measurements of apparent concentrations of 1X when the ratio varied between 0-8.0 at the fixed 1X concentration of 3×10⁻⁶ M (Table 3 below). At 1U:1X ratios of 2.5 and 3.0, the apparent increases were 22% and 32%, respectively.

TABLE 3
The effect of the ratio 1U:1X on the determination of
1X concentration by ELISA at fixed 1X concentration
of 3 × 10⁻⁶ M

| 1U:1X ratio | [1X] × 10⁶ (M) |
|---|---|
| 0.0 | 3.00 |
| 0.50 | 2.75 |
| 1.00 | 3.25 |
| 1.50 | 3.25 |
| 2.00 | 3.60 |
| 2.50 | 3.65 |
| 3.00 | 3.95 |
| 4.00 | 4.20 |
| 5.00 | 4.30 |
| 6.00 | 4.50 |
| 8.00 | 4.30 |

The following observations attested to the validity of the competitive antigen ELISA for NAT2 phenotyping.

1) The ELISA assigned the correct phenotype in 29 of 30 individuals that have been phenotyped by capillary electrophoresis (CE) (Lloyd, D. et al. (1992) J. Chrom., 578:283-291).

2) In the CE method, the phenotype was determined using AFMU/1X peak height ratios rather than the AAMU/1X molar ratios used in the ELISA. When the molar ratios determined by ELISA and the peak height ratios determined by CE were correlated by regression analysis, the calculated regression equation was y=0.48+0.87 x, with a correlation coefficient (r) of 0.84, Taking account that these two ratios are not exactly equal and that Kalow and Tang (Kalow, W. et al. (1993) Clin. Pharmacol. Ther., 53:503-514) have pointed out that using AFMU rather than AAMU can lead to misclassification of NAT2 phenotypes, there is a remarkable agreement between the two methods.

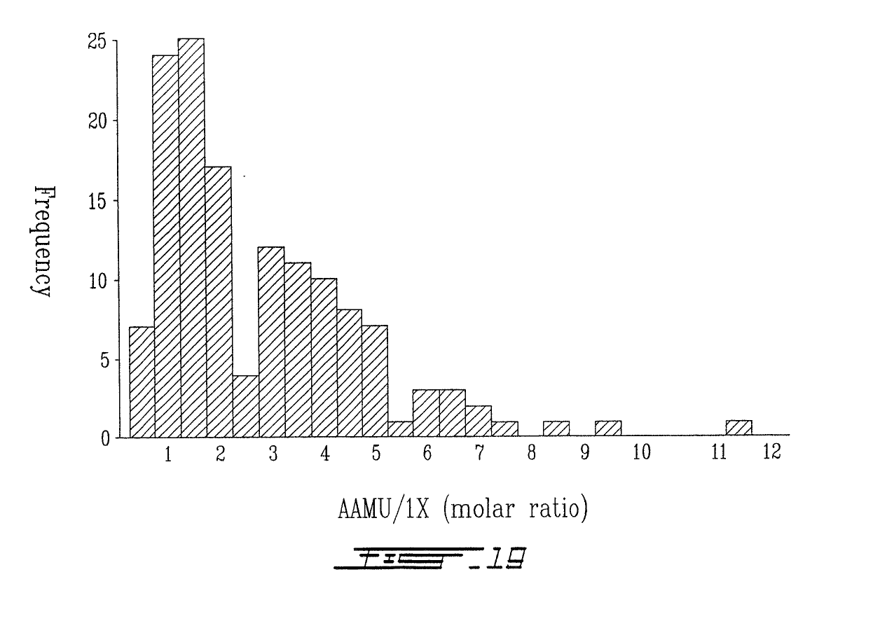

3) The ELISA was used in determining the NAT2 phenotype distribution within a group of 146 individuals. FIG. 19 illustrates a histogram of the NAT2 phenotypes of this group as determined by measuring the AAMU/1X ratio in urine samples by ELISA. Assuming an antimode of 1.80, the test population contained 60.4% slow acetylators and 39.6% fast acetylators. This is consistent with previously reported distributions (Kalow, W. et al. (1993) Clin. Pharmacol. Ther., 53:503-514; Kilbane, A. J. et al. (1990) Clin. Pharmacol. Ther., 47:470-477).

Determination of 5-acetamino-6-amino-1-methyluracyl (AAMU) and 1-methyl Xanthine in Urine Samples with the ELISA Kit

TABLE 4
Content of the ELISA kit and conditions of storage

| | | | | | Storage |
|---|---|---|---|---|---|
| Item | Unit | State | Amt | | conditions |
| Tween ™ 20 | 1 vial | Liquid | 250 | μL/vial | 4° C. |
| H₂O₂ | 1 vial | Liquid | 250 | μL/vial | 4° C. |
| AAMU-HRP | 1 vial | Liquid | 250 | μL/vial | 4° C. |
| 1X-HRP | 1 vial | Liquid | 250 | μL/vial | 4° C. |
| Buffer A | 4 vials | Solid | 0.8894 | g/vial | 4° C. |
| Buffer B | 6 vials | Solid | 1.234 | g/vial | 4° C. |
| Buffer C | 6 vials | Solid | 1.1170 | g/vial | 4° C. |
| Buffer D | 6 vials | Solid | 0.8082 | g/vial | 4° C. |
| Plate (AAMU-Ab) | 2 | Solid | — | | 4° C. |
| Plate (1X-Ab) | 2 | Solid | — | | 4° C. |
| Buffer E | 6 vials | Solid | 0.9567 | g/vial | −20° C. |
| Standards | 14 vials | Liquid | 200 | μL | −20° C. |
| (AAMU) | | | | | |
| Standards (1X) | 14 vials | Liquid | 200 | μL | −20° C. |
| 1 N NaOH | 1 bottle | Liquid | 15 | mL | 20° C. |
| 1 N HCl | 1 bottle | Liquid | 15 | mL | 20° C. |

Conversion of AFMU to AAMU

In order to determine the AAMU concentrations in urine samples by competitive antigen ELISA, a transformation of AFMU to AAMU is required. The contents of an ELISA kit for determining the AAMU concentrations are listed in Table 4.

Thaw and warm up to room temperature the urine sample.

Suspend the sample thoroughly with the vortex before pipeting.

Add 100 μL of a urine sample to a 1.5 mL-microtube.

Add 100 μL of a 1N NaOH solution.

Leave at room temperature for 10 min.

Neutralize with 100 μL 1N HCl solution.

Add 700 μL of Buffer A (dissolve the powder of one vial A/50 mL).

Dilutions of Urine Samples for the Determinations of [AAMU] and [1X] by ELISA

The dilutions of urine samples required for determinations of AAMU and 1X are a function of the sensitivity of the competitive antigen ELISA and AAMU and 1X concentrations in urine samples. It is suggested to dilute the urine samples by a factor so that AAMU and 1X concentrations are about 3×10⁻⁶ M in the well of the microtiter plate. Generally, dilution factors of 100-400 (Table 5) and 50-100 have been used for AAMU and 1X, respectively.

TABLE 5
Dilution Factors for Identifying AAMU and IX Concentrations

| | Microtube # | | | | | | | |
|---|---|---|---|---|---|---|---|---|
| Dilution Factor | 20x | 40x | 50x | 80x | 100x | 150x | 200x | 400x |
| Solution | 1 | 2 | 3 | 4 | 5 | 6 | 7 | 8 |
| Urine sample | 500 | 250 | 200 | 125 | 100 | 66.7 | 50 | 25 |
| (mL)^{a} | | | | | | | | |
| 10 × diluted | | | | | | | | |
| Buffer B (mL) | 500 | 750 | 800 | 875 | 900 | 933.3 | 950 | 975 |

^{a}Vortex the microtubes containing the urine sample before pipeting. Store the diluted urine samples at −20° C.
Buffer B: dissolve the content of one vial B/100 mL,

Precautions

The substrate is carcinogenic. Wear surgical gloves when handling Buffer E (Substrate buffer). Each sample is determined in duplicate. An excellent pipeting technique is required. When this technique is mastered the absorbance values of duplicates should be within less than 5%. Buffers C, D and E are freshly prepared. Buffer E-H₂0₂ is prepared just prior pipeting in the microtiter plate wells.

Preparation of Samples

Prepare Table 6 with a computer and print it. This table shows the content of each well of a 96-well microtiter plate. Enter the name of the urine sample (or number) at the corresponding well positions in Table 6. Select the dilution factor (D.F.) of each urine sample and enter at the corresponding position in Table 6. Enter the dilution of each urine sample with buffer B at the corresponding position in Table 6: for example, for a D.F. of 100 (100 μL of 10× diluted urine sample+900 μL buffer B), enter 100/900. See “Dilutions of urine samples . . . ” procedure described above for the preparation of the different dilutions. Prepare the different dilutions of the urine samples in 1.5-mL microtubes. Prepare Table 7 with a computer and print it. Prepare the following 48 microtubes in the order indicated in Table 7.

TABLE 6
Positions of blanks, control and urine samples in a microtiter plate

| Sample | Well # | D.F | Dil. |
|---|---|---|---|
| Blank | 1-2 | — | |
| Control | 3-4 | — | |
| S1 | 5-6 | — | |
| S2 | 7-8 | — | |
| S3 | 9-10 | — | |
| S4 | 11-12 | — | |
| S5 | 13-14 | — | |
| S6 | 15-16 | — | |
| S7 | 17-18 | — | |
| S8 | 19-20 | — | |
| S9 | 21-22 | — | |
| S10 | 23-24 | — | |
| S11 | 25-26 | — | |
| S12 | 27-28 | — | |
| S13 | 29-30 | — | |
| S14 | 31-32 | — | |
| S15 | 33-34 | — | |
| 1 | 35-36 | | |
| 2 | 37-38 | | |
| 3 | 39-40 | | |
| 4 | 41-42 | | |
| 5 | 43-44 | | |
| 6 | 45-46 | | |
| 7 | 47-48 | | |
| Control | 49-50 | | — |
| 8 | 51-52 | | |
| 9 | 53-54 | | |
| 10 | 55-56 | | |
| 11 | 57-58 | | |
| 12 | 59-60 | | |
| 13 | 61-62 | | |
| 14 | 63-64 | | |
| 15 | 65-66 | | |
| 16 | 67-68 | | |
| 17 | 69-70 | | |
| Control | 71-72 | | — |
| 18 | 73-74 | | |
| 19 | 75-76 | | |
| 20 | 77-78 | | |
| 21 | 79-80 | | |
| 22 | 81-82 | | |
| 23 | 83-84 | | |
| 24 | 85-86 | | |
| 25 | 87-88 | | |
| 26 | 89-90 | | |
| 27 | 91-92 | | |
| 28 | 93-94 | | |
| Blank | 95-96 | | — |

TABLE 7
Content of the different microtubes

| Tube # | Sample | Content | Tube # | Sample | Content |
|---|---|---|---|---|---|
| 1 | Blank | Buffer B | 25 | 7 | Dil. Urine |
| 2 | Control | Buffer B | 26 | 8 | Dil. Urine |
| 3 | S1 | AAMU or 1X | 27 | 9 | Dil. Urine |
| 4 | S2 | AAMU or 1X | 28 | 10 | Dil. Urine |
| 5 | S3 | AAMU or 1X | 29 | 11 | Dil. Urine |
| 6 | S4 | AAMU or 1X | 30 | 12 | Dil. Urine |
| 7 | S5 | AAMU or 1X | 31 | 13 | Dil. Urine |
| 8 | S6 | AAMU or 1X | 32 | 14 | Dii. Urine |
| 9 | S7 | AAMU or 1X | 33 | 15 | Dil. Urine |
| 10 | S8 | AAMU or 1X | 34 | 16 | Dil. Urine |
| 11 | S9 | AAMU or 1X | 35 | 17 | Dil. Urine |
| 12 | S10 | AAMU or 1X | 36 | Control | Buffer B |
| 13 | S11 | AAMU or 1X | 37 | 18 | Dil. Urine |
| 14 | S12 | AAMU or 1X | 38 | 19 | Dil. Urine |
| 15 | S13 | AAMU or 1X | 39 | 20 | Dil. Urine |
| 16 | S14 | AAMU or 1X | 40 | 21 | Dil. Urine |
| 17 | S15 | AAMU or 1X | 41 | 22 | Dil. Urine |
| 18 | 1 | Dil. Urine | 42 | 23 | Dil. Urine |
| 19 | 2 | Dil. Urine | 43 | 24 | Dil. Urine |
| 20 | 3 | Dil. Urine | 44 | 25 | Dil. Urine |
| 21 | 4 | Dil. Urine | 45 | 26 | Dil. Urine |
| 22 | 5 | Dil. Urine | 46 | 27 | Dil. Urine |
| 23 | 6 | Dil. Urine | 47 | 28 | Dil. Urine |
| 24 | Control | Buffer B | 48 | Blank | Buffer B |

SOLUTIONS:

| | |
|---|---|
| Buffer A: | Dissolve the content of one vial A/50 mL |
| | water. |
| Buffer B: | Dissolve the content of one vial B/100 mL |
| | water. |
| Buffer C: | Dissolve the content of one vial C/50 mL |
| | water. Add 25 mL of Tween ™ 20. |
| Buffer D: | Dissolve the content of one vial D/25 mL |
| | water. Add 25 mL of Tween ™ 20. |
| 0.05% Tween ™ 20: | Add 25 μL of Tween ™ 20 to a 100-mL |
| | erlenmeyer flask containing 50 mL of |
| | water. |
| 2.5 N HCl: | 41.75 mL of 12 N HCl/200 mL water. Store |
| | in a 250-mL glass bottle. |
| AAMU-HRP conjugate: | Add 9 mL of Buffer C to a 15-mL glass |
| | test tube. Add 90 μL of AAMU-HRP stock |
| | solution. |
| 1X-HRP conjugate: | Add 9 mL of a 2% BSA solution to a |
| | 15-mL glass test tube. Add 90 μL 1X-HRP |
| | stock solution. |
| Buffer E-H₂O₂: | Dissolve the content of one vial E- |
| | substrate/50 ml water. Add 25 μL of a |
| | 30% H₂O₂ solution (prepared just |
| | prior to adding to the microtiter |
| | plate wells). |

TABLE 8
Standard solutions of AAMU and 1X
(diluted with buffer B)

| | |
|---|---|
| AAMU | 1X |

| Standard | [AAMU] | Standard | [1X] |
|---|---|---|---|
| 1 | 1.12 × 10⁻⁴ M | 1 | 2.00 × 10⁻⁴ M |
| 2 | 6.00 × 10⁻⁵ M | 2 | 1.12 × 10⁻⁴ M |
| 3 | 3.56 × 10⁻⁵ M | 3 | 6.00 × 10⁻⁵ M |
| 4 | 2.00 × 10⁻⁵ M | 4 | 3.56 × 10⁻⁵ M |
| 5 | 6.00 × 10⁻⁶ M | 5 | 2.00 × 10⁻⁵ M |
| 6 | 3.56 × 10⁻⁶ M | 6 | 1.12 × 10⁻⁵ M |
| 7 | 2.00 × 10⁻⁶ M | 7 | 6.00 × 10⁻⁶ M |
| 8 | 1.12 × 10⁻⁶ M | 8 | 3.56 × 10⁻⁶ M |
| 9 | 6.00 × 10⁻⁷ M | 9 | 2.00 × 10⁻⁶ M |
| 10 | 3.56 × 10⁻⁷ M | 10 | 1.12 × 10⁻⁶ M |
| 11 | 2.00 × 10⁻⁷ M | 11 | 6.00 × 10⁻⁷ M |
| 12 | 1.12 × 10⁻⁷ M | 12 | 3.56 × 10⁻⁷ M |
| 13 | 6.00 × 10⁻⁸ M | 13 | 2.00 × 10⁻⁷ M |
| 14 | 3.56 × 10⁻⁸ M | 14 | 1.12 × 10⁻⁷ M |
| 15 | 2.00 × 10⁻⁸ M | 15 | 6.00 × 10⁻⁸ M |

Conditions of the ELISA

Add 50 μL/well of AAMU-HRP (or 1X-HRP) conjugate solution, starting from the last row. Add 50 μL/well of diluted urine samples in duplicate, standards (see Table 8), blank with a micropipet (0-200 μL), starting from well # 96 (see Table 6). Cover the plate and mix gently by vortexing for several seconds. Leave the plate at room temperature for 3 h. Wash 3 times with 100 μL/well with buffer C, using a microtiter plate washer. Wash 3 times with 100 μL/well with the 0.05% Tween™ 20 solution. Add 150 μL/well of Buffer E-H₂O₂ (prepared just prior adding to the microtiter plate wells). Shake 20-30 min at room temperature with an orbital shaker. Add 50 μL/well of a 2.5 N HCl solution. Shake 3 min with the orbital shaker at room temperature. Read the absorbance of the wells with microtiter plate reader at 490 nm. Print the sheet of data and properly identify the data sheet.

Calculation of the [AAMU] and [1X] in Urine Samples from the Data

Draw a Table 9 with a computer. Using the data sheet of the microtiter plate reader, enter the average absorbance values of blanks, controls (no free hapten present), standards and samples in Table 9. Draw the calibration curve on a semi-logarithmic plot (absorbance at 490 nm as a function of the standard concentrations) using sigma plot (or other plot software). Find the [AAMU] (or [1X]) in the microtiter well of the unknown from the calibration curve and enter the data in Table 10. Multiply the [AAMU] (or [1X]) of the unknown by the dilution factor and enter the result in the corresponding case of Table 10.

The compositions of the buffers used in the ELISA kit are shown in Table 11.

TABLE 9
Average absorbance values of samples in the microtiter plate

| Sample | Well # | A₄₉₀ |
|---|---|---|
| Blank | 1-2 | |
| Control | 3-4 | |
| S1 | 5-6 | |
| S2 | 7-8 | |
| S3 | 9-10 | |
| S4 | 11-12 | |
| S5 | 13-14 | |
| S6 | 15-16 | |
| S7 | 17-18 | |
| S8 | 19-20 | |
| S9 | 21-22 | |
| S10 | 23-24 | |
| S11 | 25-26 | |
| S12 | 27-28 | |
| S13 | 29-30 | |
| S14 | 31-32 | |
| S15 | 33-34 | |
| 1 | 35-36 | |
| 2 | 37-38 | |
| 3 | 39-40 | |
| 4 | 41-42 | |
| 5 | 43-44 | |
| 6 | 45-46 | |
| 7 | 47-48 | |
| Control | 49-50 | |
| 8 | 51-52 | |
| 9 | 53-54 | |
| 10 | 55-56 | |
| 11 | 57-58 | |
| 12 | 59-60 | |
| 13 | 61-62 | |
| 14 | 63-64 | |
| 15 | 65-66 | |
| 16 | 67-68 | |
| 17 | 69-70 | |
| Control | 71-72 | |
| 18 | 73-74 | |
| 19 | 75-76 | |
| 20 | 77-78 | |
| 21 | 79-80 | |
| 22 | 81-82 | |
| 23 | 83-84 | |
| 24 | 85-86 | |
| 25 | 87-88 | |
| 26 | 89-90 | |
| 27 | 91-92 | |
| 28 | 93-94 | |
| Blank | 95-96 | |

TABLE 10
AAMU (or 1X) concentrations in urine samples

| | | | |
|---|---|---|---|
| Sample | D.F. | [AAMU] | [AAMU] × D.F. |

```
  1
  2
  3
  4
  5
  6
  7
  8
  9
10
11
12
13
14
15
16
17
18
19
20
21
22
23
24
25
26
27
28
29
```

TABLE 11
Compositions of the different buffers

| | | | Concen. | [P] |
|---|---|---|---|---|
| Buffer | pH | Composition | (mM) | (mM) |
| A | 7.50 | 0.15629 g/100 mL NaH₂PO₄ | 11.325 | |
| | | 1.622 g/100 mL Na₂HPO₄.7H₂O | 60.099 | |
| | | 1.778 g/100 mL (total weight) | 71.424 | |
| B | 7.50 | 0.1210191 g/100 mL NaH₂PO₄ | 8.769 | |
| | | 1.11309 g/100 mL of | 41.23 | |
| | | Na₂HPO₄.7H₂O | | |
| | | 1.2341 g/100 mL (total weight) | 49.999 | |
| C | 7.50 | 1 g/100 mL of BSA | — | |
| | | 0.1210191 g/100 mL of NaH₂PO₄ | 8.769 | |
| | | 1.11309 g/100 mL of | 41.23 | |
| | | Na₂HPO₄.7H₂O | | |
| | | 2.2341 g/100 mL (total weight) | 49.999 | |
| D | 7.50 | 2 g/100 mL of BSA | | |
| | | 0.1210191 g/100 mL of NaH₂PO₄ | 8.769 | |
| | | 1.11309 g/100 mL of | 41.23 | |
| | | Na₂HPO₄.7H₂O | | |
| | | 3.2341 g/100 mL (total weight) | 49.999 | |
| E | 5.00 | 0.52508 g/100 mL of citric acid | 25 | |
| | | 1.34848 g/100 mL of | 50 | |
| | | Na₂HPO₄.7H₂O | | |
| | | 40 mg/100 mL of | | |
| | | o-phenylenediamine hydrochloride | | |
| | | 1.913567 g/100 mL (total weight) | | — |

The ELISA protocol outlined hereinabove, is adapted to provide a CYP3A4-specific ELISA, as well as other cytochrome P450 enzymes and N-acetylation enzymes of interest. In the case of CYP3A4, a CYP3A4-specific ELISA is provided for rapidly and accurately identifying CYP3A4 phenotypic determinants of an individual for use in treating that individual with a dosage of an Alzheimer's disease agent that is specific to at least their CYP3A4 phenotype.

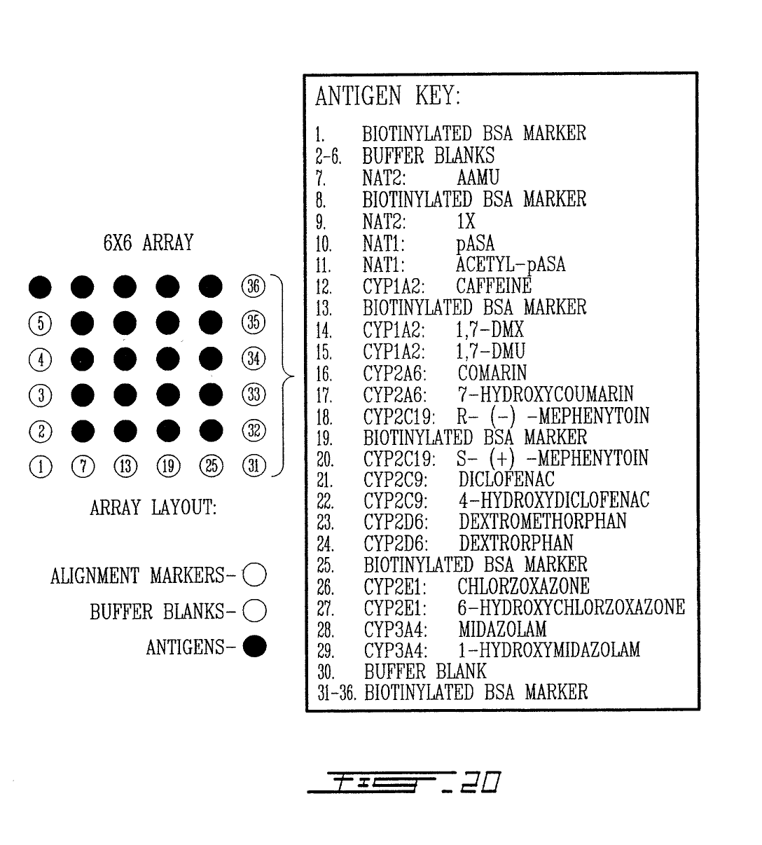

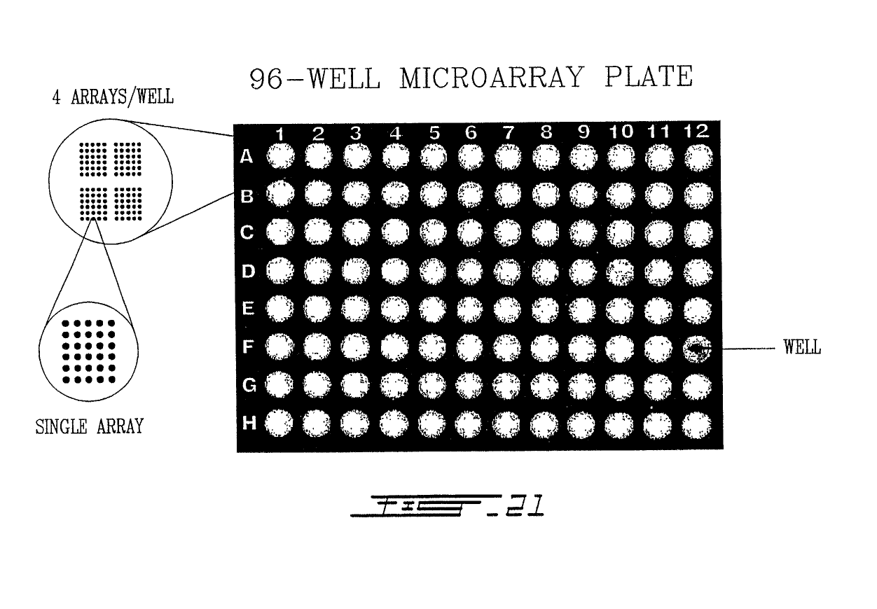

FIG. 20 exemplifies a multi-determinant assay according to an embodiment of the present invention. A multi-determinant assay of the present invention may provide more than one 6×6 array, as illustrated in FIG. 21, in each well of a standard microplate. Preferably, each well will be provided with 4 6×6 arrays according to this aspect of the present invention.

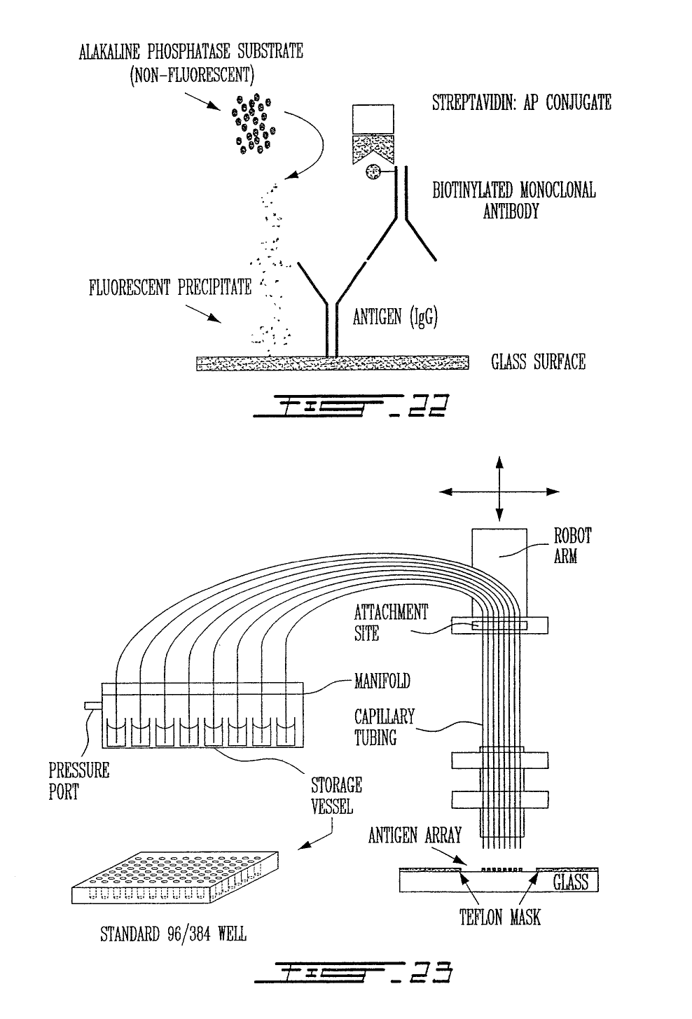

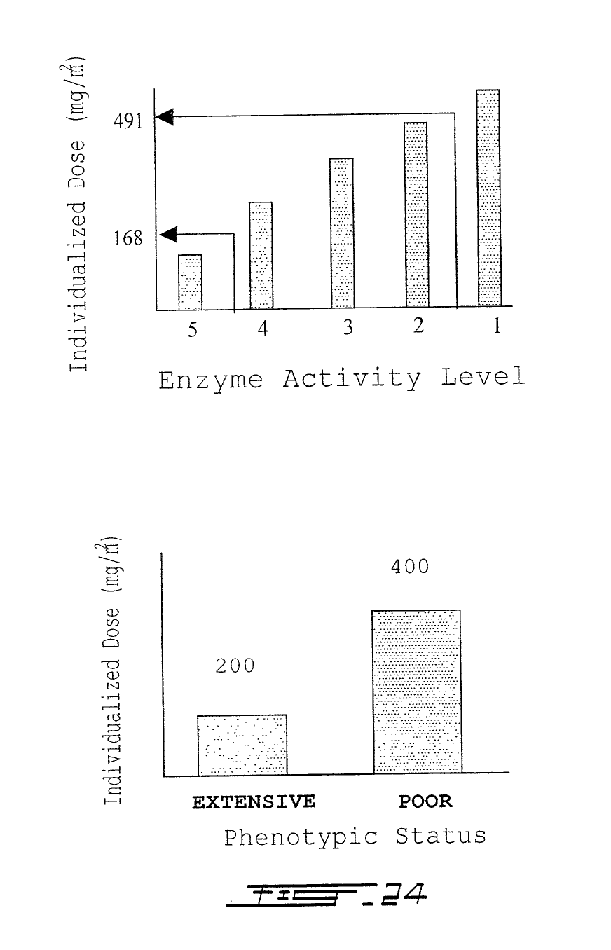

The single or multi-determinant assay system of the present invention include(s) metabolite-specific binding agents for the detection of drug-specific metabolites in a biological sample. Such binding agents are preferably antibodies and the assay system is preferably an ELISA, as exemplified in the cases of NAT2 discussed herein above. A detection method according to an embodiment of the present invention is exemplified in FIG. 22. An assay system of the present invention is exemplified in FIG. 23 and provides means to detect metabolites specific to the metabolic pathway(s) used to metabolize Alzheimer's disease agent.

The present invention provides a convenient and effective tool for use in both a clinical and laboratory environment. The present invention is particularly suited for use by a physician in a clinic, whereby phenotypic determinants for at least CYP3A4 can be quickly and easily obtained. According to an embodiment of the present invention, a ready-to-use kit is provided for fast and accurate determination of at least CYP3A4 determinants. The assay system and kit preferably employ antibodies specific to a plurality of metabolites on a suitable substrate allowing for detection of the preferred metabolites in a biological sample of an individual after consumption of a corresponding probe substrate. In accordance with a preferred embodiment of the present invention, the kit of the present invention will provide means to determine metabolic determinants for at least CYP3A4. Alternatively, the kit of the present invention will provide means for determining phenotypic determinants of CYP3A4 and at least one of the following enzymes, CYP1A2, N-acetyltransferase-1 (NAT-1), N-acetyltransferase-2 (NAT-2), CYP2D6, CYP2A6, CYP2E1, CYP2C9 and CYP2C19. The assay system of the present invention may be provided in a plurality of forms including but not limited to an ELISA assay, a high-throughput ELISA assay or a dipstick based ELISA assay.

### EXAMPLE II

### Use of Metabolic Phenotyping in Determining Individualized Treatment Regimes with Alzheimer's Disease Agent

The exposure of an individual to a drug is described by the concept of area-under-the curve (commonly referred to as AUC). AUC is related to clearance by the following equation:

AUC=dose/clearance

Thus, if an individual's clearance is known, the dose can be individualized to achieve a desired AUC by the equation:

Dose=desired AUC×clearance

An individual's rate of drug clearance is important as it determines the circulating drug concentrations. Both efficacy and toxicity are determined, in part, by the circulating concentrations of drug

Therefore, to individualize therapy a model is developed encompassing the numerous factors, which could possibly play a role in an individual's clearance value for a particular medication(s) and hence predict a dose with maximal efficacy and minimal toxicity. As drug metabolism is the principal determinant of circulating drug concentrations, determining an individual's rate of drug metabolism is an important factor for the development of a successful model for the individualization of therapy. The model of the present invention will account for an individual's rate of CYP3A4 metabolism in determining a specific dose of an Alzheimer's disease agent for that individual.

Other factors can alter drug clearance, such as body surface area, hepatic enzyme and protein levels (including serum alanine aminotransferases (ALT), albumin, alkaline phosphatases and serum α-1-acidicglycoprotein (AAG)), and drug transport proteins (including P-glycoprotein (pgp)).

Other individual specific characteristics may play a role in determining individual dose-limiting toxicity. According to another aspect of the present invention, other influencing factors may be accounted for, in addition to the rate of metabolism, in the model for the individualization of therapy with Alzheimer's disease agent. For example, in the case of many chemotherapeutic drugs, myelosuppression is the dose-limiting toxicity, and hence an individual's pretreatment white blood cell (WBC) count could be an important factor in predicting toxicity.

Using multivariate analysis these individual factors will be examined for correlation to efficacy and toxicity. In accordance with one embodiment of the present invention, factors identified as having a significant correlation to either efficacy or toxicity will be included in the model along with drug metabolism.

The importance of drug metabolism in determining an individual's rate of drug clearance renders it as the most important factor in determining the efficacy and toxicity of many drugs. Some of the metabolic enzymes mentioned in the context of this invention have a clear bimodal distribution of metabolism, allowing the separation of the population into poor and extensive metabolizers. However, within each phenotypic group there is a wide variation in metabolic rates. It may be a naïve to regard all individuals with metabolic ratios greater than a predetermined cut off value as being equivalent. This attempt to classify the population in two or three phenotypic groups is even more difficult for enzymes without a bimodal distribution. The classification of individuals into this limited classification may not allow for the complete exploitation of an individual's pattern of metabolism. In some cases this simple classification is sufficient. For example, some individuals may have an enzyme specific deficiency, such as CYP2D6 and as a result are at risk for severe complications if high doses of a particular drug, such as Prozac™ are prescribed. However, this simple classification would not allow for differential dosing of the extensive metabolizers as a function of the molar ratio calculated during determination of phenotype. If the simple classification of extensive CYP2D6 metabolizers was used, all individuals with a molar ratio of >0.3 (dextromethorphan as probe substrate) would receive the same dose. We are proposing the development of a dosing scale that would produce an increasing dose with increasing metabolic ratio, as exemplified in FIG. 24. If only the bimodal distribution is considered, only two possible doses can be prescribed. Accordingly an embodiment of the present invention, current non-individualized or categorical treatment based on phenotype can be replaced with individualization of treatment whereby the metabolism of each individual is assessed on an individual basis and a corresponding individual dosage is determined according to an individual's specific rate of metabolism for an agent or drug of interest. In this manner, Alzheimer's disease agent may be prescribed on an individual basis in dosages corresponding to at least an individual's phenotypic ability for metabolism.

In some cases multiple enzymes play key roles in determining the rate of drug metabolism. Therefore, the monitoring of only one metabolic enzyme in such cases may not provide complete information for individualizing therapy. The use of a multi-determinant assay examines multiple enzymes to provide additional metabolism-related information thereby providing a more accurate model for individualizing therapy is generated. As one drug or drug metabolite can be acted on by several enzymes (for example, clozaril by CYP1A2 and CYP2D6), the use of a multi-determinant assay, which measures the rates of multiple enzyme metabolisms, may, in some cases provide a more accurate model.

Individuals with extreme metabolic phenotypes are often at high risks for either toxicity or inefficacy of therapy. These ultraextensive or extremely poor metabolizers can often be identified by genotyping. For several metabolic enzymes genetic polymorphisms exist which result in an enzyme deficiency or the production enzyme with null activity. These individuals will not be affected by enzyme inducers or inhibitors and will consistently be extremely poor metabolizers. Identifying those individuals who carry these genetic polymorphisms allows physicians to avoid prescribing a drug metabolized by the enzyme in question. Conversely, several genetic polymorphisms have been identified that result in high levels of enzyme and/or increased enzyme activity. In addition, some individuals have been identified with multiple copies of the gene containing the polymorphism. As for the extremely poor metabolizers, these individuals may be excluded from certain treatment regimes due to increased risk of toxicity or lack of response.

Therefore, the use of genotyping to identify which individuals should be treated with a particular drug may be an excellent precursor to individualizing the individual's therapy based upon their specific phenotype. In doing so, an individual having a specific allelic variation corresponding to an enzyme specific inefficiency in metabolism can be identified before undergoing preliminary phenotyping procedures and treatment with a probe substrate or substrate.

The knowledge of an individual's (multiple) phenotypic profile will allow physicians to:

1) determine if the individual has a phenotype that allows for the safe prescription of a drug;

2) determine the optimal drug dose in terms of drug efficiency and drug safety for an individual;

3) determine which drug of a plurality of drugs used for treating an individual's pathology or condition is the optimal drug in terms of drug efficiency and drug safety for that individual.

The knowledge of an individual's phenotypic profile for one or more enzymes will allow for the detection of drug(s) that could cause significant side effects or be inefficient in individuals with a specific phenotypic profile. In addition, the phenotypic profile will allow the development of an individualized dosing scheme with dose related to level of enzyme activities. The implementation of the multi-determinant phenotyping profile in treatment and dosing selection will lead to a marked decrease in side effects and increase in therapeutic efficiency.

Donepezil

Donepezil HCl is a reversible inhibitor of the enzyme acetylcholinesterase, known chemically as (±)-2,3-dihydro-5,6-dimethoxy-2-[[1-(phenylmethyl)-4-piperidinyl]methyl] -1H-inden-1-onehydrochloride. Donepezil HCl is commonly referred to in the pharmacological literature as E2020. It has an empirical formula of C₂₄H₂₉NO₃HCl and a molecular weight of 415.96. Donepezil HCl is a white crystalline powder and is freely soluble in chloroform, soluble in water and in glacial acetic acid, slightly soluble in ethanol and in acetonitrile, and practically insoluble in ethyl acetate and in n-hexane.

Aricept is available for oral administration in film-coated tablets containing 5 or 10 mg of donepezil HCl.

Clinical Pharmacology

Current theories on the pathogenesis of the cognitive signs and symptoms of Alzheimer's disease attribute some of them to a deficiency of cholinergic neurotransmission. Donepezil HCl is postulated to exert its therapeutic effect by enhancing cholinergic function. This is accomplished by increasing the concentration of acetylcholine through reversible inhibition of its hydrolysis by acetylcholinesterase. If this proposed mechanism of action is correct, donepezil's effect may lessen as the disease process advances and fewer cholinergic neurons remain functionally intact. There is no evidence that donepezil alters the course of the underlying dementing process.

Pharmacokinetics

Donepezil is well absorbed with a relative oral bioavailability of 100%. It reaches peak plasma concentrations in 3-4 hours. Pharmacokinetics are linear over a dose range of 1-10 mg given once daily. Neither food nor time of administration (morning vs. evening dose) influences the rate or extent of absorption. The elimination half-life of donepezil is about 70 hours, and the mean apparent plasma clearance (Cl/F) is 0.13 L/hr/kg. Following multiple-dose administration, donepezil accumulates in plasma by 4- to 7-fold, and steady state is reached within 15 days. The steady state volume of distribution is 12 L/kg. Donepezil is approximately 96% bound to human plasma proteins, mainly to albumins (about 75%) and alpha1-125acid glycoprotein (about 21%) over the concentration range of 2-1000 ng/ml.

Donepezil is both excreted in the urine intact and extensively metabolized to 4 major metabolites, 2 of which are known to be active, and a number of minor metabolites, not all of which have been identified. Donepezil is metabolized by CYP 450 isoenzymes 2D6 and 3A4 and undergoes glucuronidation. Following administration of ¹⁴C-labeled donepezil, plasma radioactivity, expressed as a percent of the administered dose, was present primarily as intact donepezil (53%) and as 6-O-desmethyl donepezil (11%), which has been reported to inhibit AChE to the same extent as donepezil in vitro and was found in plasma at concentrations equal to about 20% of donepezil. Approximately 57 and 15% of the total radioactivity was recovered in urine and feces, respectively, over a period of 10 days, while 28% remained unrecovered, with about 17% of the donepezil dose recovered in the urine as unchanged drug.

Warnings

Anesthesia

Donepezil HCl, as a cholinesterase inhibitor, is likely to exaggerate succinylcholine-type muscle relaxation during anesthesia.

Cardiovascular Conditions

Because of their pharmacological action, cholinesterase inhibitors may have vagotonic effects on heart rate (e.g., bradycardia). The potential for this action may be particularly important to individuals with “sick sinus syndrome” or other supraventricular cardiac conduction conditions. Syncopal episodes have been reported in association with the use of donepezil HCl.

Gastrointestinal Conditions

Through their primary action, cholinesterase inhibitors may be expected to increase gastric acid secretion due to increased cholinergic activity. Therefore, individuals should be monitored closely for symptoms of active or occult gastrointestinal bleeding, especially those at increased risk for developing ulcers (e.g., those with a history of ulcer disease or those receiving concurrent nonsteroidal antiinflammatory drugs (NSAIDS)). Clinical studies of donepezil HCl have shown no increase, relative to placebo, in the incidence of either peptic ulcer disease or gastrointestinal bleeding.

Donepezil HCl as a predictable consequence of its pharmacological properties, has been shown to produce diarrhea, nausea, and vomiting. These effects, when they occur, appear more frequently with the 10 mg/day dose than with the 5 mg/day dose. In most cases, these effects have been mild and transient, sometimes lasting 1-3 weeks, and have resolved during continued use of donepezil HCl.

Genitourinary

Although not observed in clinical trials of donepezil HCl, cholinomimetics may cause bladder outflow obstruction.

Neurological Conditions

Seizures: Cholinomimetics are believed to have some potential to cause generalized convulsions. However, seizure activity also may be a manifestation of Alzheimer's disease.

Pulmonary Conditions

Because of their cholinomimetic actions, cholinesterase inhibitors should be prescribed with care to individuals with a history of asthma or obstructive pulmonary disease.

The most common adverse events, defined as those occurring at a frequency of at least 5% in individuals receiving 10 mg/day and twice the placebo rate, are largely predicted by donepezil HCl's cholinomimetic effects. These include nausea, diarrhea, insomnia, vomiting, muscle cramp, fatigue, and anorexia. These adverse events were often of mild intensity and transient, resolving during continued donepezil HCl treatment without the need for dose modification.

There is evidence to suggest that the frequency of these common adverse events may be affected by the rate of titration. An open-label study was conducted with 269 individuals who received placebo in the 15- and 30-week studies. These individuals were titrated to a dose of 10 mg/day over a 6-week period. The rates of common adverse events were lower than those seen in individuals titrated to 10 mg/day over 1 week in the controlled clinical trials and were comparable to those seen in individuals on 5 mg/day.

Body as a Whole: Frequent: Influenza, chest pain, toothache; Infrequent: Fever, edema face, periorbital edema, hernia hiatal, abscess, cellulitis, chills, generalized coldness, head fullness, listlessness.

Cardiovascular System: Frequent: Hypertension, vasodilation, atrial fibrillation, hotflashes, hypotension; Infrequent: Angina pectoris, postural hypotension, myocardial infarction, AV block (first degree), congestive heart failure, arteritis, bradycardia, peripheral vascular disease, supraventricular tachycardia, deep vein thrombosis.

Digestive System: Frequent: Fecal incontinence, gastrointestinal bleeding, bloating, epigastric pain; Infrequent: Eructation, gingivitis, increased appetite, flatulence, periodontal abscess, cholelithiasis, diverticulitis, drooling, dry mouth, fever sore, gastritis, irritable colon, tongue edema, epigastric distress, gastroenteritis, increased transaminases, hemorrhoids, ileus, increased thirst, jaundice, melena, polydypsia, duodenal ulcer, stomach ulcer.

Endocrine System: Infrequent: Diabetes mellitus, goiter.

Hemic and Lymphatic System: Infrequent: Anemia, thrombocythemia, thrombocytopenia, eosinophilia, erythrocytopenia.

Metabolic and Nutritional Disorders: Frequent: Dehydration; Infrequent: Gout, hypokalemia, increased creatinine kinase, hyperglycemia, weight increase, increased lactate dehydrogenase.

Musculoskeletal System: Frequent: Bone fracture; Infrequent: Muscle weakness, muscle fasciculation.

Nervous System: Frequent: Delusions, tremor, irritability, paresthesia, aggression, vertigo, ataxia, increased libido, restlessness, abnormal crying, nervousness, aphasia; Infrequent: Cerebrovascular accident, intracranial hemorrhage, transient ischemic attack, emotional lability, neuralgia, coldness (localized), muscle spasm, dysphoria, gait abnormality, hypertonia, hypokinesia, neurodermatitis, numbness (localized), paranoia, dysarthria, dysphasia, hostility, decreased libido, melancholia, emotional withdrawal, nystagmus, pacing.

Respiratory System: Frequent: Dyspnea, sore throat, bronchitis; Infrequent: Epistaxis, post nasal drip, pneumonia, hyperventilation, pulmonary congestion, wheezing, hypoxia, pharyngitis, pleurisy, pulmonary collapse, sleep apnea, snoring.

Skin and Appendages: Frequent: Pruritis, diaphoresis, urticaria; Infrequent: Dermatitis, erythema, skin discoloration, hyperkeratosis, alopecia, fungal dermatitis, herpes zoster, hirsutism, skin striae, night sweats, skin ulcers.

Special Senses: Frequent: Cataract, eye irritation, vision blurred; Infrequent: Dry eyes, glaucoma, earache, tinnitus, blepharitis, decreased hearing, retinal hemorrhage, otitis externa, otitis media, bad taste, conjunctival hemorrhage, ear buzzing, motion sickness, spots before eyes.

Urogenital System: Frequent: Urinary incontinence, nocturia; Infrequent: Dysuria, hematuria, urinary urgency, metrorrhagia, cystitis, enuresis, prostate hypertrophy, pyelonephritis, inability to empty bladder, breast fibroadenosis, fibrocystic breast, mastitis, pyuria, renal failure, vaginitis.

Postintroduction Reports

Voluntary reports of adverse events temporally associated with donepezil HCl that have been received since market introduction and are not listed above, and where there is inadequate data to determine the causal relationship with the drug include: abdominal pain, agitation, cholecystitis, confusion, convulsions, hallucinations, heart block (all types), hemolytic anemia, hepatitis, hyponatremia, pancreatitis, and rash.

The present invention provides for an individualization model based upon at least an individual's specific CYP3A4 phenotype for use in the individualization of therapy with Alzheimer's disease agent. This proactive procedure will identify starting doses much more accurately than the standard methods, and will result in much less post-administration “fine-tuning” of the dose.

In accordance with one embodiment of the present invention, prior to undergoing treatment with an Alzheimer's disease agent individuals are administered a predetermined dose of a CYP3A4 specific probe substrate. A biological sample is collected (e.g. urine) after the probe substrate is consumed. The concentrations of the probe substrate and metabolite(s) are determined and a molar ratio calculated. This molar ratio is specific to the individual's level of CYP3A4 activity.

To determine the rate of CYP3A4 activity, midazolam may be used as a probe substrate and the molar ratio of the midazolam metabolite and midazolam (1′-hydroxymidazolam/midazolam) calculated. An individual's ratio is indicative of their CYP3A4 enzyme activity, with a lower ratio indicating poorer metabolism and a higher ratio indicating more extensive metabolism. The activity of CYP3A4 metabolism is distributed unimodally and hence no antimode is present.

In accordance with another embodiment of the present invention, prior to undergoing treatment with an Alzheimer's disease agent, individuals are administered a predetermined dose of a CYP2D6 specific probe substrate. A biological sample is collected (e.g. urine) after the probe substrate is consumed. The concentrations of the probe substrate and metabolite(s) are determined and a molar ratio calculated. This molar ratio is specific to the individual's level of CYP2D6 activity.

To determine the rate of CYP2D6 activity dextromethorphan may be used as a probe drug and the molar ratio of dextromethorphan and the dextromethorphan derivative (dextrorphan) calculated. An individual's ratio will be considered as indicative of CYP2D6 activity with a ratio of less than 0.30 indicative of an extensive metabolizer and greater than 0.30 indicative of a poor metabolizer.

The levels of CYP3A4 or CYP2D6 activity as determined by direct phenotyping will be incorporated into an individualization of therapy model of the present invention to determine a treatment dosage of an Alzheimer's disease agent that correlates with an individual's ability to metabolize that Alzheimer's disease agent. An ELISA system as exemplified above may be employed to detect phenotypic determinants of at least CYP3A4 or at least CYP2D6 for determining an individual's CYP3A4 or CYP2D6 metabolic activity, respectively. The present invention provides for an individualization model based upon at least an individual's specific CYP3A4 or CYP2D6 phenotype for use in the individualization of therapy with Alzheimer's disease agent. The individualization model of the present invention may further include other enzyme-specific determinants as well as other factors, which have a significant contribution to the clearance of Alzheimer's disease agent in the body or a significant contribution to toxicity (e.g. pretreatment renal function).

In accordance with an embodiment of the present invention, an assay system is provided that can be used in a clinical environment, whereby phenotypic determinants can be quantified from a urine sample and applied to an individualization model to determine a dosage of an Alzheimer's disease agent for treating an individual which at least corresponds to the individual's ability to metabolize CYP3A4 or CYP2D6 substrates. As a result, physicians will be provided with a tool for the individualization of therapy providing an alternative to the arbitrary selection of medications based on prognosis and categorical dosing.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method of characterizing a multi-determinant metabolic phenotype for at least one Alzheimer's disease agent, wherein a plurality of phenotypic determinants are identified as corresponding to respective metabolic characteristics; said method comprising:
a) administering to an individual a probe substrate specific to metabolic pathway(s) for said at least one Alzheimer's disease agent;
b) detecting metabolites of said metabolic pathway(s) in a biological sample from said individual in response to said probe substrate; and
c) characterizing respective phenotypic determinants of said multi-determinant metabolic phenotype based on detected metabolites.

2. The method of claim 1, wherein said at least one Alzheimer's disease agent is selected from the group consisting of acetylcholinesterase inhibitor, nicotinic compound, gamma secretase inhibitor, M2 antagonist.

3. The method of claim 1, wherein said at least one Alzheimer's disease agent is donezepil.

4. The method of claim 1 which further comprises a step i) after step b):
i) quantifying a ratio of respective detected metabolites for each of said metabolic pathways in said biological sample.

5. The method of claim 4, wherein said ratio is selected from the group consisting of concentration ratio, molar ratio, chiral ratio, ratio of area under the curve and signal peak height ratio.

6. The method of claim 1, wherein said probe substrate is at least one probe substrate known to be metabolized by said metabolic pathway.

7. The method of claim 1, wherein said probe substrate is other than an inducer or inhibitor of said metabolic pathway.

8. The method of claim 1, wherein said step b) or step c) is effected using an affinity complexation agent specific to each of said metabolites.

9. The method of claim 8, wherein said affinity complexation agent is an antibody.

10. The method of claim 9, wherein said antibody is a monoclonal antibody.

11. The method of claim 9, wherein said antibody is a polyclonal antibody.

12. The method of claim 8, wherein said affinity complexation agent is a molecular imprinted polymer.

13. The method of claim 8, wherein said affinity complexation agent is an aptmer.

14. The method of claim 8, wherein said affinity complexation agent is a receptor.

15. The method of claim 8, wherein said affinity complexation agent is an anticalin.

16. The method of claim 1, further comprising a ligand binding assay.

17. The method of claim 16, wherein said ligand binding assay is selected from the group consisting of immunoassay, enzyme-linked immunosorbent assay (ELISA), microarray formatted immunoassay and microarray formatted ELISA.

18. The method of claim 16, wherein said ligand binding assay is a rapid immunoassay (Dipstick assay).

19. The method of claim 18, wherein said rapid immunoassay is based on Rapid Analyte Measurement Platform (RAMP) technology.

20. The method of claim 18, wherein said rapid immunoassay is based on light-emitting immunoassay technology.

21. The method of claim 16, wherein said ligand binding assay is performed with a biosensor.

22. The method of claim 21, wherein said biosensor is an immunosensor.

23. The method of claim 21 wherein wherein the means of detection of said biosensor is an electrochemical sensor.

24. The method of claim 21, wherein the means of detection of said biosensor is an optical sensor.

25. The method of claim 21, wherein the means of detection of said biosensor is a microgravimetric sensor.

26. The method of claim 25, wherein said microgravimetric sensor is a quartz crystal microbalance (QCM).

27. The method of claim 1, wherein step b) is effected by using a qualitative detection instrument.

28. The method according to claim 1, wherein each of said plurality of phenotypic determinants of said multi-determinant metabolic phenotype is an enzyme-specific determinant.

29. The method according to claim 1, wherein said multi-determinant metabolic phenotype is comprised of at least one determinant indicative of an individual's metabolic capacity for at least one drug metabolizing enzyme.

30. The method of claim 29, wherein said at least one drug metabolizing enzyme is CYP3A4.

31. The method of claim 30, further comprising at least one drug metabolizing enzyme selected from the group consisting of N-acetyltransferase-1 (NAT-1), N-acetyltransferase-2 (NAT-2), CYP1A2, CYP2D6, CYP2A6, CYP2E1, CYP2C9, CYP2C19, UGTs, GSTs, and STs.

32. The method of claim 1, wherein step a) is effected by using a plurality of probe substrates and wherein each probe substrate is specific to at least one metabolic pathway of interest.

33. The method of claim 1, further comprising:
d) measuring at least one determinant for drug clearance known to affect the toxicity or efficacy of said at least one Alzheimer's disease agent compound; wherein said at least one determinant is factored together with at least rate of probe substrate metabolism to determine a non-toxic and effective amount of said at least one Alzheimer's disease agent compound to be administered to said individual.

34. The method of claim 33, wherein said at least one determinant for drug clearance is based on body surface area or hepatic enzyme levels of said individual.

35. The method of claim 1, further comprising:
d) measuring at least one determinant for drug susceptibility known to affect the toxicity or efficacy of said at least one Alzheimer's disease agent compound; wherein said at least one determinant for drug susceptibility is factored together with at least rate of probe substrate metabolism to determine a non-toxic and effective amount of said at least one Alzheimer's disease agent compound to be administered to said individual.

36. The method of claim 35, wherein said at least one determinant for drug susceptibility is based on pretreatment renal function of said individual determined prior to step a).

37. The method of claim 35, wherein said at least one Alzheimer's disease agent is selected from the group consisting of acetylcholinesterase inhibitor, nicotinic compound, gamma secretase inhibitor, M2 antagonist.

38. The method of claim 35, wherein said at least one Alzheimer's disease agent is donezepil.

39. The method of claim 36, further comprising:
e) measuring at least one determinant for drug susceptibility known to affect the toxicity or efficacy of said at least one Alzheimer's disease agent compound; wherein said at least one determinant for drug susceptibility is factored together with at least rate of probe substrate metabolism to determine a non-toxic and effective amount of said at least one Alzheimer's disease agent compound to be administered to said individual.

40. The method of claim 39, wherein said at least one determinant for drug susceptibility is based on pretreatment renal function of said individual determined prior to step a).

41. The method of claim 39, wherein said at least one Alzheimer's disease agent is selected from the group consisting of an acetylcholinesterase inhibitor, a nicotinic compound, a gamma secretase inhibitor, a M2 antagonist.

42. The method of claim 39, wherein said at least one Alzheimer's disease agent is donezepil.

43. A method of using a multi-determinant metabolic phenotype to individualize a treatment regimen for at least one Alzheimer's disease agent compound for an individual, wherein the multi-determinant metabolic phenotype of said individual is determined; a safe and therapeutically effective dose of said at least one Alzheimer's disease agent compound treatment is determined and/or selected based on said multi-determinant metabolic phenotype of said individual.

44. The method of claim 43, wherein said at least one Alzheimer's disease agent is selected from the group consisting of an acetylcholinesterase inhibitor, a nicotinic compound, a gamma secretase inhibitor, a M2 antagonist.

45. The method of claim 43, wherein said at least one Alzheimer's disease agent is donezepil.

46. The method of claim 43, wherein said multi-determinant metabolic phenotype is determined according to the method comprising:
a) administering to an individual a probe substrate specific to metabolic pathway(s) for said at least one Alzheimer's disease agent;
b) detecting metabolites of said metabolic pathway(s) in a biological sample from said individual in response to said probe substrate; and
c) characterizing respective phenotypic determinants of said multi-determinant metabolic phenotype based on detected metabolites.

47. A method of administering to an individual at least one Alzheimer's disease agent compound, said method comprising:
a) determining a multi-determinant metabolic phenotype of said individual; and
b) administering a safe and therapeutically effective dose of said at least one Alzheimer's disease agent compound to said individual, wherein said dose has been determined based on a metabolic profile of said individual corresponding to said individual's metabolic phenotype for said at least one Alzheimer's disease agent compound as represented by said multi-determinant metabolic phenotype.

48. The method of claim 47, wherein said at least one Alzheimer's disease agent is selected from the group consisting of an acetylcholinesterase inhibitor, a nicotinic compound, a gamma secretase inhibitor, a M2 antagonist.

49. The method of claim 47, wherein said at least one Alzheimer's disease agent is donezepil.

50. The method of claim 47, wherein said multi-determinant metabolic phenotype is characterized according to the method comprising:
a) administering to an individual a probe substrate specific to metabolic pathway(s) for said at least one Alzheimer's disease agent;
b) detecting metabolites of said metabolic pathway(s) in a biological sample from said individual in response to said probe substrate; and
c) characterizing respective phenotypic determinants of said multi-determinant metabolic phenotype based on detected metabolites.

51. An assay system for detecting the presence of enzyme-specific metabolites in a biological sample, said sample obtained from an individual treated with a known amount of at least one probe substrate for at least one Alzheimer's disease agent compound, specific for metabolic pathways of said metabolites, said assay comprising:
a) means for receiving said biological sample, including a plurality of affinity complexation agents contained therein;
b) means for detecting presence of said enzyme-specific metabolites bound to said affinity complexation agents; and
c) means for quantifying ratios of said metabolites to provide corresponding phenotypic determinants;
wherein said phenotypic determinants provide a metabolic phenotypic profile of said individual.

52. The assay system of claim 51, wherein said at least one Alzheimer's disease agent is selected from the group consisting of an acetylcholinesterase inhibitor, a nicotinic compound, a gamma secretase inhibitor, a M2 antagonist.

53. The assay system of claim 51, wherein said at least one Alzheimer's disease agent is donezepil.

54. The assay system of claim 51, wherein said step b) or step c) is effected according to the method comprising:
a) administering to an individual a probe substrate specific to metabolic pathway(s) for said at least one Alzheimer's disease agent;
b) detecting metabolites of said metabolic pathway(s) in a biological sample from said individual in response to said probe substrate; and
c) characterizing respective phenotypic determinants of said multi-determinant metabolic phenotype based on detected metabolites;
wherein said probe substrate is at least one substrate known to be metabolized by said metabolic pathway, and wherein said probe substrate is other than an inducer or inhibitor of said metabolic pathway.

55. The assay system of claim 51, wherein said assay is a ligand binding assay.

56. The assay system of claim 55, wherein said ligand binding assay is selected from the group consisting of immunoassay, enzyme-linked immunosorbent assay (ELISA), microarray formatted immunoassay and microarray formatted ELISA.

57. The assay system of claim 51, wherein said means for receiving said biological sample is a multi-well microplate including said plurality of affinity complexation agents in each well.

58. The assay system of claim 57, wherein said plurality of affinity complexation agents are bound to each well in an array-based format.

59. The assay system of claim 51, wherein said means for detecting said presence of said metabolites bound to said binding agents is a charge-coupled device (CCD) imager.

60. The assay system of claim 51, wherein said means for quantifying ratios of said metabolites is a densitometer.

61. A method of using an enzyme-specific assay for the individualization of administration of at least one Alzheimer's disease agent compound, which comprises:
a) conducting said assay to identify phenotypic determinants in a biological sample obtained from an individual treated with a probe substrate for said at least one Alzheimer's disease agent compound;
b) determining a rate of drug metabolism according to said determinants; and
c) determining and/or selecting a safe and therapeutically effective dose of said class of Alzheimer's disease agent compounds for said individual based on said rate.

62. The method of claim 61, wherein said assay comprises:
a) means for receiving said biological sample, including a plurality of affinity complexation agents contained therein;
b) means for detecting presence of said enzyme-specific metabolites bound to said affinity complexation agents; and
c) means for quantifying ratios of said metabolites to provide corresponding phenotypic determinants;
wherein said phenotypic determinants provide a metabolic phenotypic profile of said individual.

63. The method of claim 61, wherein said at least one Alzheimer's disease agent is selected from the group consisting of an acetylcholinesterase inhibitor, a nicotinic compound, a gamma secretase inhibitor, a M2 antagonist.

64. The method of claim 61, wherein said at least one Alzheimer's disease agent is donezepil.

65. The method of claim 61, wherein said enzyme-specific assay is selected from the group consisting of immunoassay, enzyme-linked immunosorbent assay (ELISA), microarray formatted immunoassay and microarray formatted ELISA.

66. The method of claim 61, wherein said rate of drug metabolism corresponds to a ratio of phenotypic determinants, wherein said phenotypic determinants are enzyme-specific determinants.

67. The method of claim 66, wherein said ratio is selected from the group consisting of concentration ratio, molar ratio, chiral ratio, ratio of area under the curve and signal peak height ratio.

68. The method of claim 61, wherein said phenotypic determinants comprise phenotypic determinants for CYP3A4.

69. The method of claim 68, wherein said phenotypic determinants further comprise phenotypic determinants for any one or more of N-acetyltransferase-1 (NAT1), N-acetyltransferase-2 (NAT2), CYP1A2, CYP2A6, CYP2D6, CYP2E1, CYP2C9, and CYP2C19, UGTs, GSTs, and STs.

70. A method of screening a plurality of individuals for participation in a drug treatment trial assessing the therapeutic effect of at least one Alzheimer's disease agent compound, said method comprising:
a) selecting individuals having a metabolic phenotype characterized as effective for metabolizing said at least one Alzheimer's disease agent compound.

71. The method of claim 70, wherein said at least one Alzheimer's disease agent is selected from the group consisting of an acetylcholinesterase inhibitor, a nicotinic compound, a gamma secretase inhibitor, a M2 antagonist.

72. The method of claim 70, wherein said at least one Alzheimer's disease agent is donezepil.

73. The method of claim 70, wherein said multi-determinant metabolic phenotype is determined according to the method comprising:
a) administering to an individual a probe substrate specific to metabolic pathway(s) for said at least one Alzheimer's disease agent;
b) detecting metabolites of said metabolic pathway(s) in a biological sample from said individual in response to said probe substrate; and
c) characterizing respective phenotypic determinants of said multi-determinant metabolic phenotype based on detected metabolites.

74. A method of screening a plurality of individuals for treatment with at least one Alzheimer's disease agent compound, said method comprising:
a) genotyping said individuals to identify individuals lacking at least one allelic variation known to prompt toxicity of said at least one Alzheimer's disease agent compound;
and
b) selecting individuals having a metabolic phenotype characterized as effective for metabolizing said at least one Alzheimer's disease agent compound.

75. The method of claim 74, further comprising determining a safe and therapeutically effective amount of said at least one Alzheimer's disease agent compound to be administered to each of said individuals lacking said at least one allelic variation, said effective amount corresponding to an individual-specific rate of drug metabolism as determined by phenotypic determinants specific for at least one enzyme known to metabolize said at least one Alzheimer's disease agent compound.

76. The method of claim 74, wherein said step of characterizing a metabolic phenotype comprises a ligand-binding assay specific for said at least one enzyme known to metabolize said at least one Alzheimer's disease agent compound.

77. The method of claim 74, wherein said at least one Alzheimer's disease agent is selected from the group consisting of an acetylcholinesterase inhibitor, a nicotinic compound, a gamma secretase inhibitor, a M2 antagonist.

78. The method of claim 74, wherein said at least one Alzheimer's disease agent is donezepil.

79. The method of claim 76, wherein said ligand-binding assay is selected from the group consisting of immunoassay, enzyme-linked immunosorbent assay (ELISA), microarray formatted immunoassay and microarray formatted ELISA.

80. The method of claim 75, wherein said rate of drug metabolism corresponds to a ratio of phenotypic determinants for at least CYP3A4 enzyme.

81. The method of claim 80, wherein said ratio is selected from the group consisting of concentration ratio, molar ratio, chiral ratio, ratio of area under the curve and signal peak height ratio.

82. The method of claim 76, wherein said ligand-binding assay further provides means to determine phenotypic determinants for at least one of the following enzymes: NAT2, CYP1A2, NAT1, CYP2A6, CYP2D6, CYP2E1, CYP2C9 and CYP2C19, CYP3A4, UGTs, GSTs, and STs.

83. A method of screening a plurality of individuals for participation in a drug treatment trial assessing the therapeutic effect of a candidate Alzheimer's disease agent compound treatment, said method comprising:
a) genotyping each of said individuals to identify individuals lacking at least one allelic variation known to prompt the toxicity of said Alzheimer's disease agent compound;
and
b) characterizing a multi-determinant metabolic phenotype of said identified individuals of step a) to determine each individual's ability to metabolize said Alzheimer's disease agent compound.

84. The method of claim 83, wherein said at least one Alzheimer's disease agent is selected from the group consisting of an acetylcholinesterase inhibitor, a nicotinic compound, a gamma secretase inhibitor, a M2 antagonist.

85. The method of claim 83, wherein said at least one Alzheimer's disease agent is donezepil.

86. The method of claim 83, wherein said multi-determinant metabolic phenotype is comprised of at least one determinant indicative of an individual's metabolic capacity for at least one drug metabolizing enzyme.

87. The method of claim 86, wherein said at least one drug metabolizing enzyme is selected from the group consisting of N-acetyltransferase-1 (NAT1), N-acetyltransferase-2 (NAT2), CYP1A2, CYP2A6, CYP2D6, CYP2E1, CYP3A4, CYP2C9, CYP2C19, UGTs, GSTs, and ST.

88. The method of claim 87, wherein said rate of drug metabolism corresponds to a ratio of said phenotypic determinants for said at least one enzyme.

89. The method of claim 88, wherein said ratio is selected from the group consisting of concentration ratio, molar ratio, chiral ratio, ratio of area under the curve and signal peak height ratio.

90. The method of claim 4, wherein said step b) or step c) is effected using an affinity complexation agent specific to each of said metabolites.

91. The method of claim 1, wherein said step b) and step c) are effected using an affinity complexation agent specific to each of said metabolites.

92. The method of claim 4, wherein said step b) and step c) are effected using an affinity complexation agent specific to each of said metabolites.

93. The assay system of claim 51, wherein said step b) and step c) are effected according to the method comprising:
a) administering to an individual a probe substrate specific to metabolic pathway(s) for said at least one Alzheimer's disease agent;
b) detecting metabolites of said metabolic pathway(s) in a biological sample from said individual in response to said probe substrate; and
c) characterizing respective phenotypic determinants of said multi-determinant metabolic phenotype based on detected metabolites;
wherein said probe substrate is at least one substrate known to be metabolized by said metabolic pathway, and wherein said probe substrate is other than an inducer or inhibitor of said metabolic pathway.


SEQUENCE LISTING (25 sequences)

SEQ ID NO: 1 - DNA, 20 bp, Artificial Sequence
  Primer
gctgggtctggaagctcctc

SEQ ID NO: 2 - DNA, 22 bp, Artificial Sequence
  Primer
ttgggtgatacatacacaaggg

SEQ ID NO: 3 - DNA, 21 bp, Artificial Sequence
  Primer
tcctagaagacagcaacgacc

SEQ ID NO: 4 - DNA, 18 bp, Artificial Sequence
  Primer
gtgaagcccaccaaacag

SEQ ID NO: 5 - DNA, 22 bp, Artificial Sequence
  Primer
ggccatctttaaaatacatttt

SEQ ID NO: 6 - DNA, 31 bp, Artificial Sequence
  Primer
cctcccttgctggctgtgtcccaagctaggc

SEQ ID NO: 7 - DNA, 31 bp, Artificial Sequence
  Primer
cgccccttcctttccgccatcctgcccccag

SEQ ID NO: 8 - DNA, 20 bp, Artificial Sequence
  Primer
gcgtggtattcagcaacggg

SEQ ID NO: 9 - DNA, 19 bp, Artificial Sequence
  Primer
tgccccgtggaggttgacg

SEQ ID NO: 10 - DNA, 20 bp, Artificial Sequence
  Primer
aattacaaccagagcttggc

SEQ ID NO: 11 - DNA, 22 bp, Artificial Sequence
  Primer
tatcactttccataaaagcaag

SEQ ID NO: 12 - DNA, 20 bp, Artificial Sequence
  Primer
aacatcaggattgtaagcac

SEQ ID NO: 13 - DNA, 20 bp, Artificial Sequence
  Primer
tcagggcttggtcaatatag

SEQ ID NO: 14 - DNA, 24 bp, Artificial Sequence
  Primer
caatggaaagaaatggaaggaggt

SEQ ID NO: 15 - DNA, 24 bp, Artificial Sequence
  Primer
agaaagtaatactcagaccaatcg

SEQ ID NO: 16 - DNA, 20 bp, Artificial Sequence
  Primer
tgcacgaggtccagagatgc

SEQ ID NO: 17 - DNA, 27 bp, Artificial Sequence
  Primer
agcttcagggtttacgtatcatagtaa

SEQ ID NO: 18 - DNA, 22 bp, Artificial Sequence
  Primer
ccagaaggctttgcaggcttca

SEQ ID NO: 19 - DNA, 22 bp, Artificial Sequence
  Primer
actgagccctgggaggtaggta

SEQ ID NO: 20 - DNA, 23 bp, Artificial Sequence
  Primer
ccatttggtagtgaggcaggtat

SEQ ID NO: 21 - DNA, 23 bp, Artificial Sequence
  Primer
caccatccatgtttgcttctggt

SEQ ID NO: 22 - DNA, 18 bp, Artificial Sequence
  Primer
cccgtgagccagtcgagt

SEQ ID NO: 23 - DNA, 18 bp, Artificial Sequence
  Primer
atacagaccctcttccac

SEQ ID NO: 24 - DNA, 21 bp, Artificial Sequence
  Primer
agtcgacatgtgatggatcca

SEQ ID NO: 25 - DNA, 21 bp, Artificial Sequence
  Primer
gacagggtttcatcatgttgg